(19) United States
(12) Patent Application Publication
(10) Pub. No.: US 2026/0226187 A1
(43) Pub. Date: Aug. 6, 2026

(54) CONDITIONALLY ACTIVE ANTI-EPCAM ANTIBODIES, ANTIBODY FRAGMENTS AND CONSTRUCTS INCORPORATING SAME

(71) Applicant: BIOATLA, INC., San Diego, CA (US)

(72) Inventors: Jay M. Short, Jackson, WY (US); Gerhard Frey, San Diego, CA (US); Hwai Wen Chang, San Marcos, CA (US)

(73) Assignee: BIOATLA, INC., San Diego, CA (US)

(21) Appl. No.: 19/159,783

(22) PCT Filed: Mar. 1, 2024
(86) PCT No.: PCT/US2024/017940

§ 371 Date: Aug. 26, 2025

Related U.S. Application Data
(60) Provisional application No. 63/488,153, filed on Mar. 2, 2023.

(51) Int. Cl. C07K 16/30; C07K 16/28

(52) U.S. Cl. CPC C07K 16/30; C07K 16/2809; C07K 2317/31; C07K 2317/33; C07K 2317/565; C07K 2317/622; C07K 2317/73; C07K 2317/92

(57) ABSTRACT

A conditionally active bispecific antibody comprising an IgG antibody or antibody fragment that binds to a human EpCAM protein comprising a light chain variable region having three complementarity determining regions L1, L2 and L3; a heavy chain variable region having three complementarity determining regions H1, H2 and H3; and at least one scFv antibody fragment that binds to a T-lymphocyte protein linked to a C-terminus of at least one light chain of the IgG antibody or antibody fragment.

### FIELD OF THE DISCLOSURE

This disclosure relates to anti-EpCAM antibodies, antibody fragments and constructs incorporating the anti-EpCAM antibodies and antibody fragments such as multi-specific antibodies and conjugates. Also disclosed are diagnostic and therapeutic uses of the antibodies, antibody fragments and constructs.

### BACKGROUND OF THE DISCLOSURE

Epithelial cell adhesion/activating molecule (EpCAM, also known as CD326, HEA125, MK-1, EGP-2, EGP34, GA733-2, KSA, TROP-1, KS1/4 and ESA)(FIG. 1) is one of the first and most important immunotherapeutic targets in cancer therapy, due to its high-level and frequent expression on most carcinomas of different origin (Herlyn et al., Proc Natl Acad Sci USA, 76:1438-1442, 1979; Went et al., Hum Pathol, 35:122-128, 2004). This molecule is a relatively small type I transmembrane glycoprotein of 314 amino acids (aa) in length that is highly conserved during evolution. It is reported to mediate calcium-independent homotypic cell-cell adhesions (Litvinov et al., J Cell Biology, 125:437-446, 1994). The molecule consists of a short intracellular domain of 26 aa in which two binding sites for α-actinin are present for interaction with the actin cytoskeleton (Balzar et al., Mol Cell Biol., 18(8): 4833-4843, 1998), a 23-aa transmembrane domain, a 242-aa extracellular domain (ECD), and a 23-aa signal peptide which is cleaved from its mature form. The extracellular domain of EpCAM has three N-linked glycosylation sites. Differential glycosylation status between normal and malignant tissues has been reported in certain types of cancer (Pauli et al., Cancer Lett, 193:25-32, 2003).

The extracellular domain contains 3 domains. The first two are believed to resemble epidermal growth factor (EGF)-like repeats in which twelve cysteine residues exist among them (Balzar et al., Mol Cell Biol, 21:2570-2580, 2001). However, some studies suggest that the second EGF-like repeat of EpCAM is in fact a thyroglobulin (TY) domain (Linnenbach et al., Proc Natl Acad Sci USA, 86:27-31, 1989; Chong and Speicher, J Biol Chem, 276:5804-5813, 2001). The third domain is a unique cysteine-poor region (CPR) unrelated to any known molecules (Baeuerle and Gires, Br J Cancer, 96:417-423, 2007). EpCAM plays an important role in the prevention of cell-cell adhesion, cell signalling, migration, proliferation and differentiation (FIG. 1).

EpCAM expression in humans is epithelia-specific. The majority of human epithelial cells express EpCAM, except squamous epithelium and some specific epithelial cell types, such as epidermal keratinocytes, hepatocytes, gastric parietal cells, and myoepithelial cells (Balzar et al., J Mol Med, 77: 699-712, 1999; Momburg et al., Cancer Res, 47:2883-2891, 1987). In tumors of epithelial origin, generally a higher expression level is observed (Balzar et al., J Mol Med, 77: 699-712, 1999; Winter et al., Am J Pathol, 163:2139-2148, 2003; Went et al., Hum Pathol, 35:122-128, 2004; Went et al., Br J Cancer, 94:128-135, 2006). For example, it has been found that the EpCAM protein is expressed on a great variety of human adenocarcinomas and squamous cell carcinomas (Went et al., Hum Pathol, 35:122-128, 2004). Recent studies using immunohistochemistry (IHC) staining together with microarrays technology has discovered EpCAM expression in a fairly large number of samples from patients with breast, ovarian, renal, esophageal, colon, gastric, prostate and lung cancer (Spizzo et al., Breast Cancer Res Treat, 86:207-213, 2004; Spizzo et al., Gynecol Oncol, 103:483-488, 2006; Stoecklein et al., BMC Cancer, 6:165, 2006; Kimura et al., Int J Oncol, 30:171-179, 2007; Went et al., Am J Surg Pathol, 29:83-88, 2005; Went et al., Br J Cancer, 94:128-135, 2006). The data underscore the potential utility of EpCAM as an immunotherapeutic target for treatment of human cancers.

Since epithelial cells are known to be the most important cell type in the development of human malignancies, and more than 90% of all malignant tumors are of epithelial origin (Birchmeiera et al., Acta Anatomica, 156 (3):217-226, 1996), EpCAM is now considered to be one of the most frequently and intensely expressed tumor-associated antigens. The molecule has many times been independently discovered as an immunogenic tumor-associated antigen for development of monoclonal antibodies (Gottlinger et al., Int J Cancer, 38:47-53, 1986; Edwards et al., Cancer Res, 46:1306-1317, 1986; Spurr et al., Int J Cancer, 38:631-636, 1986; Momburg et al., Cancer Res, 47:2883-2891, 1987; Schön et al., J Investig Dermatol, 102: 987-991, 1994; Bumol et al., Hybridoma, 7:407-415, 1988; Quak et al., Hybridoma, 9:377-387, 1990).

Indeed, the first monoclonal antibody ever applied for human cancer therapy was in fact a murine IgG2a antibody called mAb 17-1A (later named edrecolomab and Panorexs) which targets EpCAM (Sears et al., Lancet, 1(8275):762-765, 1982; Sears et al., J Biol Response Mod, 3(2):138-150, 1984). Since then, edrecolomab and other EpCAM-specific murine, chimeric and humanized monoclonal antibodies were also tested pre-clinically and clinically either in the form of native (naked) antibody, hybrid bispecific (trifunctional) antibody or as conjugates with toxins, radioisotopes, or cytokines (IL-2 or GM-CSF) for cancer treatment (Velders et al., Cancer Res, 54(7):1753-1759, 1994; Raum et al., Cancer Immunol Immunother, 50(3):141-150, 2001; Elias et al., Am J Respir Crit Care Med, 150:1114-1122, 1994; Di Paolo et al., Clin Cancer Res, 9:2837-2848, 2003; Andratschke et al., Anticancer Res, 27(1A):431-436, 2007; Xiang et al., Cancer Res, 57(21):4948-4955, 1997; Schanzer et al., J Immunother, 29(5):477-488, 2006; Wimberger et al., Int J Cancer, 105(2):241-248, 2003; Amann et al., Cancer Res, 68(1):143-151, 2008). To date numerous different immunotherapeutic approaches targeting EpCAM are still currently in clinical trials (Baeuerle and Gires, Br J Cancer, 96:417-423, 2007). Data from clinical trials have suggested that naked anti-EpCAM antibodies such as edrecolomab (17-1A; Panorexs) and adecatumumab (MT201) have only limited anti-tumor effect (Punt et al., Lancet, 360: 671-677, 2002), likely through the activation of the complement system (CDC) and the antibody-dependent cytotoxicity (ADCC) effect (Schwartzberg, Crit Rev Oncol Hematol, 40(1):17-24, 2001; Naundorf et al., Int J Cancer, 100(1):101-110, 2002; Prang et al., Br J Cancer, 92(2):342-349, 2005; Oberneder et al., Eur J Cancer, 42(15):2530-2538, 2006). Antibodies conjugated with very potent effector mechanisms such as IL-2, PE toxin, or anti-CD3 seem to have a better anti-tumor effect. However, some adverse effects limit the systemic use of such anti-EpCAM antibodies (Baeuerle and Gires, Br J Cancer, 96:417-423, 2007).

ING-1 is a high affinity human engineered monoclonal antibody that targets EpCAM positive cells. It has been used in a phase I clinical trial in patients with advanced adenocarcinomas, refractory to standard therapy and the data from this study suggested that antibodies with high affinity to EpCAM, while being more cytotoxic to tumor cells, can also induce rapid pancreatic toxic injury thus, limiting their therapeutic window for systemic administration (De Bono et al., Clin Cancer Res, 10(22):7555-65, 2004). The possible systemic toxic effects associated with the therapeutic use of high affinity anti-EpCAM antibodies, might be reduced by pre-targeting strategies which include a chasing step to eliminate, at a given time, the circulating antibody. Alternatively, the use of high affinity anti-EpCAM antibodies might be restricted to loco-regional treatments.

The side effects of known anti-EpCAM antibodies are associated with the presence of EpCAM on normal epithelial cells, albeit with a lower density compared to tumor cells (Kim et al., Clin Cancer Res, 10:5464-5471, 2004; Osta et al., Cancer Res, 64:5818-5824, 2004). Thus, increasing the affinity or specificity of the anti-EpCAM antibodies does not lead to a reduction of the effects of the anti-EpCAM antibodies in the normal tissues that express EpCAM, which produces the side effects.

The present disclosure aims at providing anti-EpCAM antibodies or antibody fragments with reduced or minimal side effects suitable for therapeutic and diagnostic use, especially for diagnosis and treatment of cancers. Some of these anti-EpCAM antibodies or antibody fragments may have a higher binding affinity to EpCAM in a tumor in comparison with EpCAM present in normal tissues. These anti-EpCAM antibodies or antibody fragments typically have at least comparable efficacy to known anti-EpCAM antibodies. In addition, the present anti-EpCAM antibodies or antibody fragments may exhibit reduced side effects in comparison with monoclonal anti-EpCAM antibodies known in the art for having a relatively low binding affinity to EpCAM in normal tissues. These advantages may provide a more selective targeting of the EpCAM for a tumor and may permit use of higher dosages of these anti-EpCAM antibodies or antibody fragments due to the selectivity of the antibodies for EpCAM present in a tumor, whereby more effective therapeutic treatments can be realized without a corresponding increase in undesirable side effects.

Antibodies have become a major class of therapeutic proteins. Traditional antibodies normally bind to a single epitope on an antigen. New antibody constructs, referred to as multi-specific antibodies, have been developed for binding to more than one antigen or to more than one epitope on the same antigen. Multi-specific antibodies may be, for example, bispecific, tri-specific, or tetra-specific antibodies. Multi-specific antibodies have shown potential in a broad range of clinical and diagnostic applications. There are two bispecific antibody drugs approved in the European Union and United States for treatment of oncological diseases (Catumaxomab™ and Blinatumab™). Due to their unique features, multi-specific antibodies have become attractive for next generation antibody therapeutics.

US 2013/0017200 discloses a method of synthesizing multi-specific antibodies. A first antibody fragment is obtained from a first parent antibody having a first mono-specificity, which has a free sulfhydryl group that may be reacted with a thio-reactive crosslinker to produce an antibody fragment-crosslinker moiety. The antibody fragment-crosslinker moiety is reacted pairwise with each of two or more additional antibody fragments obtained from other parent antibodies having a mono-specificity that is different from the first antibody fragment, each having a free sulfhydryl group, to produce the multi-specific antibodies. The multi-specific antibodies may be suitable as new therapeutic and diagnostic agents.

Brinkmann and Kontermann (“The making of bispecific antibodies,” MABS, 2017, vol. 9, pp. 182-212, 2017) surveys formats of bispecific antibodies, including small molecules composed solely of the antigen binding sites of two antibodies, molecules with an IgG structure, and large complex molecules composed of different antigen-binding moieties often combined with dimerization modules. Depending on different applications, the bispecific antibodies may vary in size, arrangement, valence, flexibility and geometry of their binding modules, as well as in their distribution and pharmacokinetic properties. The bispecific formats collectively increase the diversity of the antibodies that can be applied to the development of therapeutics for various indications. Examples of particular bispecific formats are found in FIGS. 2 and 3.

It is also desirable to generate useful antibodies that are conditionally active. For example, antibodies virtually inactive at a normal physiological condition and significantly more active at a condition other than the normal physiological condition (e.g., an aberrant condition), antibodies that are activated or inactivated in certain microenvironments (e.g., activated in a tumor microenvironment), or antibodies that are activated or inactivated over time. Besides temperature, other trigger conditions for which the antibodies can be evolved or optimized include pH, osmotic pressure, osmolality, oxidative stress and electrolyte concentration. Besides the activity, other desirable properties of antibodies that can be optimized during evolution include stability, half-life, chemical resistance, and proteolytic resistance.

Many strategies for evolving or engineering a parent antibody to mutant antibodies having a desired property have been published. However, engineering or evolving a parent antibody to be inactive or virtually inactive (less than 10% activity and especially less than 5% activity) at the normal physiological condition, while having substantial activity at an aberrant condition at the normal physiological condition, requires that destabilizing mutation(s) co-exist with activity increasing mutations that do not counter the destabilizing effect. It is expected that destabilizing mutations would reduce the antibody's activity by an amount greater than is predicted by standard rules such as the Q10 rule. Therefore, the ability to evolve proteins that work efficiently (more active) at a specific aberrant condition, e.g. a lower temperature or pH, while being substantially inactive at their normal operating condition, creates a surprising new class of antibodies referred to as conditionally active antibody.

Among the embodiments disclosed herein is a new class of multi-specific antibodies that are conditionally active for binding to the EpCAM antigen. This new class of multi-specific antibodies can take advantage of the flexibility and versatility of traditional multi-specific antibodies, and while at the same time directing the activity, affinity and/or avidity of the multi-specific antibodies to locations, tissues or organs of a subject where the activity is desired. In doing so, these multi-specific antibodies avoid or greatly reduce adverse effects associated with binding to normal cells, including, but not limited to, avoidance of excess cytokine production.

### SUMMARY OF THE DISCLOSURE

Provided herein are conditionally active antibodies or antibody fragments that bind to a human EpCAM protein or an epitope of an EpCAM protein comprising a light chain variable region and a heavy chain variable region, wherein the light chain variable region includes three complementarity determining regions L1, L2 and L3, and the heavy chain variable region includes three complementarity determining regions HI, H2 and H3. The complementarity determining regions of the various embodiments of the conditionally active anti-EpCAM antibodies and antibody fragments disclosed herein are provided in Table 1

TABLE 1
Complementarity Determining Regions of Embodiments of the Present Disclosure

| | SEQ ID NO. | | | | | |
|---|---|---|---|---|---|---|
| Antibody | CDR-L1 | CDR-L2 | CDR-L3 | CDR-H1 | CDR-H2 | CDR-H3 |
| A | 7 | 2 | 3 | 4 | 5 | 6 |
| B | 8 | 2 | 3 | 4 | 5 | 6 |
| C | 9 | 2 | 3 | 4 | 5 | 6 |
| D | 10 | 2 | 3 | 4 | 5 | 6 |
| E | 1 | 11 | 3 | 4 | 5 | 6 |
| F | 1 | 12 | 3 | 4 | 5 | 6 |
| G | 1 | 13 | 3 | 4 | 5 | 6 |
| H | 1 | 14 | 3 | 4 | 5 | 6 |
| I | 1 | 15 | 3 | 4 | 5 | 6 |
| J | 1 | 16 | 3 | 4 | 5 | 6 |
| K | 1 | 17 | 3 | 4 | 5 | 6 |
| L | 1 | 18 | 3 | 4 | 5 | 6 |
| M | 1 | 19 | 3 | 4 | 5 | 6 |
| N | 1 | 20 | 3 | 4 | 5 | 6 |
| O | 1 | 2 | 21 | 4 | 5 | 6 |
| P | 1 | 2 | 22 | 4 | 5 | 6 |
| Q | 1 | 2 | 23 | 4 | 5 | 6 |
| R | 1 | 2 | 3 | 24 | 5 | 6 |
| S | 1 | 2 | 3 | 25 | 5 | 6 |
| T | 1 | 2 | 3 | 26 | 5 | 6 |
| U | 1 | 2 | 3 | 27 | 5 | 6 |
| V | 1 | 2 | 3 | 28 | 5 | 6 |
| W | 1 | 2 | 3 | 29 | 5 | 6 |
| X | 1 | 2 | 3 | 30 | 5 | 6 |
| Y | 1 | 2 | 3 | 31 | 5 | 6 |
| Z | 1 | 2 | 3 | 4 | 32 | 6 |
| AA | 1 | 2 | 3 | 4 | 33 | 6 |
| AB | 1 | 2 | 3 | 4 | 34 | 6 |
| AC | 1 | 2 | 3 | 4 | 35 | 6 |
| AD | 1 | 2 | 3 | 4 | 36 | 6 |
| AE | 1 | 2 | 3 | 4 | 37 | 6 |
| AF | 1 | 2 | 3 | 4 | 38 | 6 |
| AG | 1 | 2 | 3 | 4 | 5 | 39 |
| AH | 1 | 2 | 3 | 4 | 5 | 40 |
| AI | 1 | 2 | 3 | 4 | 5 | 41 |
| AJ | 1 | 2 | 3 | 4 | 5 | 42 |
| AK | 1 | 2 | 3 | 4 | 5 | 43 |
| AL | 1 | 2 | 3 | 4 | 5 | 44 |
| AM | 1 | 2 | 3 | 4 | 5 | 45 |
| AN | 1 | 2 | 3 | 4 | 5 | 46 |
| AO | 1 | 2 | 3 | 4 | 5 | 47 |
| AP | 1 | 2 | 3 | 4 | 5 | 48 |
| AQ | 1 | 2 | 3 | 4 | 5 | 49 |
| AR | 1 | 2 | 3 | 4 | 5 | 50 |

In several additional embodiments the conditionally active anti-EpCAM antibodies and antibody fragments may be selected from combinations of a heavy chain variable region of SEQ ID NO: 52 and a light chain variable region of SEQ ID NO: 53, a heavy chain variable region of SEQ ID NO: 52 and a light chain variable region of SEQ ID NO: 54, a heavy chain variable region of SEQ ID NO: 52 and a light chain variable region of SEQ ID NO: 55, a heavy chain variable region of SEQ ID NO: 52 and a light chain variable region of SEQ ID NO: 56, a heavy chain variable region of SEQ ID NO: 52 and a light chain variable region of SEQ ID NO: 57, a heavy chain variable region of SEQ ID NO: 52 and a light chain variable region of SEQ ID NO: 58, a heavy chain variable region of SEQ ID NO: 52 and a light chain variable region of SEQ ID NO: 59, a heavy chain variable region of SEQ ID NO: 52 and a light chain variable region of SEQ ID NO: 60, a heavy chain variable region of SEQ ID NO: 52 and a light chain variable region of SEQ ID NO: 61, a heavy chain variable region of SEQ ID NO: 52 and a light chain variable region of SEQ ID NO: 62, a heavy chain variable region of SEQ ID NO: 52 and a light chain variable region of SEQ ID NO: 63, a heavy chain variable region of SEQ ID NO: 52 and a light chain variable region of SEQ ID NO: 64, a heavy chain variable region of SEQ ID NO: 52 and a light chain variable region of SEQ ID NO: 65, a heavy chain variable region of SEQ ID NO: 52 and a light chain variable region of SEQ ID NO: 66, a heavy chain variable region of SEQ ID NO: 52 and a light chain variable region of SEQ ID NO: 67, a heavy chain variable region of SEQ ID NO: 52 and a light chain variable region of SEQ ID NO: 68, and a heavy chain variable region of SEQ ID NO: 52 and a light chain variable region of SEQ ID NO: 69.

In further embodiments, the conditionally active anti-EpCAM antibodies and antibody fragments may include combinations selected from a light chain variable region of SEQ ID NO: 51 and a heavy chain variable region of SEQ ID NO: 70, a light chain variable region of SEQ ID NO: 51 and a heavy chain variable region of SEQ ID NO: 71, a light chain variable region of SEQ ID NO: 51 and a heavy chain variable region of SEQ ID NO: 72, a light chain variable region of SEQ ID NO: 51 and a heavy chain variable region of SEQ ID NO: 73, a light chain variable region of SEQ ID NO: 51 and a heavy chain variable region of SEQ ID NO: 74, a light chain variable region of SEQ ID NO: 51 and a heavy chain variable region of SEQ ID NO: 75, a light chain variable region of SEQ ID NO: 51 and a heavy chain variable region of SEQ ID NO: 76, a light chain variable region of SEQ ID NO: 51 and a heavy chain variable region of SEQ ID NO: 77, a light chain variable region of SEQ ID NO: 51 and a heavy chain variable region of SEQ ID NO: 78, a light chain variable region of SEQ ID NO: 51 and a heavy chain variable region of SEQ ID NO: 79, a light chain variable region of SEQ ID NO: 51 and a heavy chain variable region of SEQ ID NO: 80, a light chain variable region of SEQ ID NO: 51 and a heavy chain variable region of SEQ ID NO: 81, a light chain variable region of SEQ ID NO: 51 and a heavy chain variable region of SEQ ID NO: 82, a light chain variable region of SEQ ID NO: 51 and a heavy chain variable region of SEQ ID NO: 83, a light chain variable region of SEQ ID NO: 51 and a heavy chain variable region of SEQ ID NO: 84, a light chain variable region of SEQ ID NO: 51 and a heavy chain variable region of SEQ ID NO: 85, a light chain variable region of SEQ ID NO: 51 and a heavy chain variable region of SEQ ID NO: 86, a light chain variable region of SEQ ID NO: 51 and a heavy chain variable region of SEQ ID NO: 87, a light chain variable region of SEQ ID NO: 51 and a heavy chain variable region of SEQ ID NO: 88, a light chain variable region of SEQ ID NO: 51 and a heavy chain variable region of SEQ ID NO: 89, a light chain variable region of SEQ ID NO: 51 and a heavy chain variable region of SEQ ID NO: 90, a light chain variable region of SEQ ID NO: 51 and a heavy chain variable region of SEQ ID NO: 91, a light chain variable region of SEQ ID NO: 51 and a heavy chain variable region of SEQ ID NO: 92, a light chain variable region of SEQ ID NO: 51 and a heavy chain variable region of SEQ ID NO: 93, a light chain variable region of SEQ ID NO: 51 and a heavy chain variable region of SEQ ID NO: 94, a light chain variable region of SEQ ID NO: 51 and a heavy chain variable region of SEQ ID NO: 95, and a light chain variable region of SEQ ID NO: 51 and a heavy chain variable region of SEQ ID NO: 96.

The conditionally active antibodies or antibody fragments of the present disclosure exhibit increased binding affinity to an EpCAM protein or an epitope of an EpCAM protein at an aberrant condition which differs from a normal physiological condition and a decreased binding affinity to an EpCAM protein or an epitope of an EpCAM protein at a normal physiological condition. In one embodiment the aberrant condition is a condition in the tumor microenvironment and the normal physiological condition is a condition in the non-tumor microenvironment. In a further embodiment the condition is pH. In one particular embodiment the aberrant condition is a pH of 5.0 to 6.9 in a tumor microenvironment and the normal physiological condition is a non-tumor microenvironment pH of 7.0 to 7.6. In still another particular embodiment, the conditionally active anti-EpCAM antibodies or antibody fragments of the present disclosure have an increased binding affinity to an EpCAM protein or an epitope of an EpCAM protein at a pH of 6.0 as compared to the binding affinity of the same conditionally active anti-EpCAM antibody or antibody fragment at a pH of 7.4.

In one embodiment, the conditionally active anti-EpCAM antibodies or antibody fragments disclosed herein are obtained from a non-conditionally active anti-EpCAM parental antibody. In a particular embodiment, the non-conditionally active anti-EpCAM parental antibody has a light chain variable region of SEQ ID NO: 51 and a heavy chain variable region of SEQ ID NO: 52. In this embodiment, the conditionally active anti-EpCAM antibodies or antibody fragments disclosed herein have increased binding affinity to an EpCAM protein or an epitope of an EpCAM protein at a tumor microenvironment pH of 5.0 to 6.9 as compared to the binding affinity of the same conditionally active anti-EpCAM antibody or antibody fragment at a non-tumor microenvironment pH of 7.0 to 7.6, and a decreased binding affinity to the EpCAM protein or epitope of the EpCAM protein at pH 7.0 to 7.6 when compared to the binding affinity of the parental non-conditionally active anti-EpCAM antibody or antibody fragment to the EpCAM protein or epitope of the EpCAM protein at pH 7.0 to 7.6. In a particular embodiment, the conditionally active anti-EpCAM antibodies disclosed herein have increased binding affinity to an EpCAM protein or an epitope of an EpCAM protein at a pH of 6.0 as compared to the binding affinity of the same conditionally active anti-EpCAM antibody at a pH of 7.4, and a decreased binding affinity to the EpCAM protein or epitope of the EpCAM protein at pH 7.4 when compared to binding affinity the parental non-conditionally active anti-EpCAM antibody to the EpCAM protein or epitope of the EpCAM protein at pH 7.4.

In one embodiment, the conditionally active anti-EpCAM antibodies or antibody fragments disclosed herein have a ratio of binding affinity to an EpCAM protein or an epitope of an EpCAM protein at a pH of 6.0 to a binding affinity to the EpCAM protein or epitope of the EpCAM protein of at a pH of 7.4 of at least about 1.5:1, at least about 2:1, at least about 3:1, at least about 4:1, at least about 5:1, at least about 6:1, at least about 7:1, at least about 8:1, at least about 9:1, at least about 10:1, at least about 15:1 or at least about 20:1.

Also provided herein are multi-specific antibodies comprising a conditionally active anti-EpCAM antibody or antibody fragment of the present disclosure, and at least one scFv antibody fragment that binds to a T-lymphocyte antigen linked to a C-terminus of at least one light chain or at least one heavy chain of the conditionally active anti-EpCAM antibody or antibody fragment.

In one embodiment, the anti-EpCAM antibody or antibody fragment of the multi-specific antibody comprises a light chain variable region containing three complementarity determining regions L1, L2 and L3, and a heavy chain variable region containing three complementarity determining regions H1, H2 and H3. The light chain and heavy chain complementarity determining regions of the conditionally active anti-EpCAM antibody or antibody fragment of the multi-specific antibody can be obtained from the various embodiments disclosed in Table 1 above.

In several additional embodiments the conditionally active anti-EpCAM antibody or antibody fragment of the multi-specific antibody may include combinations of a heavy chain variable region of SEQ ID NO: 52 and a light chain variable region of SEQ ID NO: 53, a heavy chain variable region of SEQ ID NO: 52 and a light chain variable region of SEQ ID NO: 54, a heavy chain variable region of SEQ ID NO: 52 and a light chain variable region of SEQ ID NO: 55, a heavy chain variable region of SEQ ID NO: 52 and a light chain variable region of SEQ ID NO: 56, a heavy chain variable region of SEQ ID NO: 52 and a light chain variable region of SEQ ID NO: 57, a heavy chain variable region of SEQ ID NO: 52 and a light chain variable region of SEQ ID NO: 58, a heavy chain variable region of SEQ ID NO: 52 and a light chain variable region of SEQ ID NO: 59, a heavy chain variable region of SEQ ID NO: 52 and a light chain variable region of SEQ ID NO: 60, a heavy chain variable region of SEQ ID NO: 52 and a light chain variable region of SEQ ID NO: 61, a heavy chain variable region of SEQ ID NO: 52 and a light chain variable region of SEQ ID NO: 62, a heavy chain variable region of SEQ ID NO: 52 and a light chain variable region of SEQ ID NO: 63, a heavy chain variable region of SEQ ID NO: 52 and a light chain variable region of SEQ ID NO: 64, a heavy chain variable region of SEQ ID NO: 52 and a light chain variable region of SEQ ID NO: 65, a heavy chain variable region of SEQ ID NO: 52 and a light chain variable region of SEQ ID NO: 66, a heavy chain variable region of SEQ ID NO: 52 and a light chain variable region of SEQ ID NO: 67, a heavy chain variable region of SEQ ID NO: 52 and a light chain variable region of SEQ ID NO: 68, and a heavy chain variable region of SEQ ID NO: 52 and a light chain variable region of SEQ ID NO: 69.

In further embodiments, the conditionally active anti-EpCAM antibodies or antibody fragments of the multi-specific antibody contain a combination of a light chain variable region of SEQ ID NO: 51 and a heavy chain variable region of SEQ ID NO: 70, a light chain variable region of SEQ ID NO: 51 and a heavy chain variable region of SEQ ID NO: 71, a light chain variable region of SEQ ID NO: 51 and a heavy chain variable region of SEQ ID NO: 72, a light chain variable region of SEQ ID NO: 51 and a heavy chain variable region of SEQ ID NO: 73, a light chain variable region of SEQ ID NO: 51 and a heavy chain variable region of SEQ ID NO: 74, a light chain variable region of SEQ ID NO: 51 and a heavy chain variable region of SEQ ID NO: 75, a light chain variable region of SEQ ID NO: 51 and a heavy chain variable region of SEQ ID NO: 76, a light chain variable region of SEQ ID NO: 51 and a heavy chain variable region of SEQ ID NO: 77, a light chain variable region of SEQ ID NO: 51 and a heavy chain variable region of SEQ ID NO: 78, a light chain variable region of SEQ ID NO: 51 and a heavy chain variable region of SEQ ID NO: 79, a light chain variable region of SEQ ID NO: 51 and a heavy chain variable region of SEQ ID NO: 80, a light chain variable region of SEQ ID NO: 51 and a heavy chain variable region of SEQ ID NO: 81, a light chain variable region of SEQ ID NO: 51 and a heavy chain variable region of SEQ ID NO: 82, a light chain variable region of SEQ ID NO: 51 and a heavy chain variable region of SEQ ID NO: 83, a light chain variable region of SEQ ID NO: 51 and a heavy chain variable region of SEQ ID NO: 84, a light chain variable region of SEQ ID NO: 51 and a heavy chain variable region of SEQ ID NO: 85, a light chain variable region of SEQ ID NO: 51 and a heavy chain variable region of SEQ ID NO: 86, a light chain variable region of SEQ ID NO: 51 and a heavy chain variable region of SEQ ID NO: 87, a light chain variable region of SEQ ID NO: 51 and a heavy chain variable region of SEQ ID NO: 88, a light chain variable region of SEQ ID NO: 51 and a heavy chain variable region of SEQ ID NO: 89, a light chain variable region of SEQ ID NO: 51 and a heavy chain variable region of SEQ ID NO: 90, a light chain variable region of SEQ ID NO: 51 and a heavy chain variable region of SEQ ID NO: 91, a light chain variable region of SEQ ID NO: 51 and a heavy chain variable region of SEQ ID NO: 92, a light chain variable region of SEQ ID NO: 51 and a heavy chain variable region of SEQ ID NO: 93, a light chain variable region of SEQ ID NO: 51 and a heavy chain variable region of SEQ ID NO: 94, a light chain variable region of SEQ ID NO: 51 and a heavy chain variable region of SEQ ID NO: 95, and a light chain variable region of SEQ ID NO: 51 and a heavy chain variable region of SEQ ID NO: 96.

In one embodiment, the conditionally active anti-EpCAM antibodies or antibody fragments of the multi-specific antibody disclosed herein are obtained from a non-conditionally active anti-EpCAM parental antibody. In a particular embodiment, the non-conditionally active anti-EpCAM parental antibody has a light chain variable region of SEQ ID NO: 51 and a heavy chain variable region of SEQ ID NO: 52. In this embodiment, the conditionally active anti-EpCAM antibodies or antibody fragments of the multi-specific antibodies disclosed herein have increased binding affinity to an EpCAM protein or an epitope of an EpCAM protein at a tumor microenvironment pH of 5.0 to 6.9 as compared to the binding affinity of the same conditionally active anti-EpCAM antibody or antibody fragment at a non-tumor microenvironment pH of 7.0 to 7.6, and a decreased binding affinity to the EpCAM protein or epitope of the EpCAM protein at pH 7.0 to 7.6 when compared to the binding affinity of the parental non-conditionally active anti-EpCAM antibody or antibody fragment to the EpCAM protein or epitope of the EpCAM protein at pH 7.0 to 7.6. In a particular embodiment, the conditionally active anti-EpCAM antibodies of the multi-specific antibodies disclosed herein have increased binding affinity to an EpCAM protein or an epitope of an EpCAM protein at a pH of 6.0 as compared to the binding affinity of the same conditionally active anti-EpCAM antibody at a pH of 7.4, and a decreased binding affinity to the EpCAM protein or epitope of the EpCAM protein at pH 7.4 when compared to binding affinity the parental non-conditionally active anti-EpCAM antibody to the EpCAM protein or epitope of the EpCAM protein at pH 7.4.

In one embodiment, the conditionally active anti-EpCAM antibodies or antibody fragments of the multi-specific antibodies disclosed herein have a ratio of binding affinity to an EpCAM protein or an epitope of an EpCAM protein at a pH of 6.0 to a binding affinity to the EpCAM protein or epitope of the EpCAM protein of at a pH of 7.4 of at least about 1.5:1, at least about 2:1, at least about 3:1, at least about 4:1, at least about 5:1, at least about 6:1, at least about 7:1, at least about 8:1, at least about 9:1, at least about 10:1, at least about 15:1 or at least about 20:1. In one particular embodiment, the conditionally active anti-EpCAM antibodies or antibody fragments of the multi-specific antibodies disclosed herein have a ratio of binding affinity to an EpCAM protein or an epitope of an EpCAM protein at a pH of 6.0 to a binding affinity to the EpCAM protein or epitope of the EpCAM protein of at a pH of 7.4 of at least about 6:1. In one embodiment, the conditionally active anti-EpCAM antibodies or antibody fragments of the multi-specific antibodies disclosed herein have a ratio of binding affinity to an EpCAM protein or an epitope of an EpCAM protein at a pH of 6.0 to a binding affinity to the EpCAM protein or epitope of the EpCAM protein of at a pH of 7.4 of at least about 8:1.

In one embodiment, the conditionally active anti-EpCAM antibody or antibody fragment of the multi-specific antibody is an IgG antibody or antibody fragment. In another embodiment the conditionally active anti-EpCAM IgG antibody or antibody fragment is part of a bispecific antibody. In additional embodiments, the anti-lymphocyte antigen scFv antibody or antibody fragment is attached to the C-terminus of the light chain of the conditionally active anti-EpCAM IgG antibody or antibody fragment.

In certain embodiments, the scFv anti-lymphocyte antigen antibody or antibody fragment is an anti-CD3 scFv antibody. In certain embodiments, the anti-CD3 scFv antibody or antibody fragment is a conditionally active antibody or antibody fragment while in other embodiments, the anti-CD3 scFv is a non-conditionally active antibody or antibody fragment. In embodiments where the anti-CD3 scFv antibody or antibody fragment is conditionally active, the anti-CD3 scFv antibody has a higher binding affinity for a CD3 antigen at a tumor microenvironment pH of 5.0 to 6.9 as compared to the binding affinity of the same conditionally active anti-CD3 scFv antibody or antibody fragment at a non-tumor microenvironment pH of 7.0 to 7.6. In a particular embodiment, the anti-CD3 scFv antibody or antibody fragment comprises a light chain variable region of SEQ ID NO: 101 and a heavy chain variable region of SEQ ID NO: 100. In another embodiment, the anti-CD3 scFv antibody comprises SEQ ID NO: 97. In one particular embodiment, the conditionally active anti-EpCAM/anti-CD3 multi-specific antibody comprises a light chain of SEQ ID NO: 98 and a heavy chain of SEQ ID NO: 99.

In another embodiment, the conditionally active anti EpCAM antibody or antibody fragment of the multi-specific antibody binds to a cynomolgus EpCAM protein in addition to the human EpCAM protein and has a ratio of binding affinity to cynomolgus EpCAM protein at a pH of 6.0 to a binding affinity to cynomolgus EpCAM protein at a pH of 7.4 of at least about 6:1, at least about 7:1, at least about 8:1, at least about 9:1, at least about 10:1, at least about 15:1 or at least about 20:1. In one particular embodiment the anti-EpCAM antibody or antibody fragment of the multi-specific antibody has light chain variable region CDRs 1 2 and 3 of SEQ ID NO:1, SEQ ID NO: 2 and SEQ ID NO: 3, respectively, and heavy chain variable region CDRs of SEQ ID NO: 4, SEQ ID NO: 5, and SEQ ID NO: 45, respectively. In another embodiment, the anti-EpCAM antibody or antibody fragment of the multi-specific antibody has light chain variable region of SEQ ID NO: 51 and a heavy chain variable region of SEQ ID NO: 91. In any of the preceding embodiments, the multi-specific antibody can be a bispecific antibody. In certain embodiments, the bispecific antibody is an anti-EpCAM/anti-CD3 antibody. In these embodiments, the anti-CD3 antibody is a scFv antibody with a light chain variable region of SEQ ID NO: 101 and a heavy chain variable region of SEQ ID NO: 100. In a more particular embodiment, the anti-CD3 scFv antibody has an amino acid sequence of SEQ ID NO: 97. In still another embodiment, the anti-EpCAM/anti-CD3 bispecific antibody has a light chain sequence of SEQ ID NO: 98 and a heavy chain sequence of SEQ ID NO: 99.

### BRIEF DESCRIPTION OF THE DRAWINGS

FIG. 1 shows a schematic representation of the functions of EpCAM in cancer metastasis and progression.

FIG. 2 shows a schematic structure of a bi-valent multi-specific antibody that is a hetero-dimer with one arm for binding to EpCAM (Ag) and the other arm for binding to CD3.

FIG. 3 shows a schematic structure of a tetra-valent multi-specific antibody that is a homodimer with each arm having a binding site to an antigen (Ag) and a binding site to CD3.

FIGS. 4A-4B show sequence alignments of exemplary light chain variable regions of anti-EpCAM antibodies of the present disclosure.

FIGS. 5A, 5B and 5C show sequence alignments of exemplary heavy chain variable regions of anti-EpCAM antibodies of the present disclosure.

FIG. 6A shows binding affinity of BA3182 to human CD3/human EpCAM at pH 6.0 and pH 7.4. Binding of BA3182 to human CD3 and human EpCAM at pH 6.0 and pH 7.4 was measured by ELISA. Average of OD values from 2 replicates at pH 6.0 and pH 7.4 were plotted against different concentrations of BA3182. The figure shows the dose-response binding curves of BA3182 to human CD3/human EpCAM antigens at different pH values. Data is representative of 3 experiments. Y-axis: OD 450 nm. X-axis: concentration of BA3182.

FIG. 6B shows binding affinity of BA3182 to human CD3/cyno EpCAM at pH 6.0 and pH 7.4. Binding of BA3182 to human CD3 and cyno EpCAM at pH 6.0 and pH7.4 was measured by ELISA. Average of OD values from 2 replicates at pH 6.0 and pH 7.4 were plotted against different concentrations of BA3182. The figure shows the dose-response binding curves of BA3182 to human CD3/cyno EpCAM antigens at different pH values. Data is representative of 3 experiments. Y-axis: OD 450 nm. X-axis: concentration of BA3182.

FIG. 7 shows binding of BA3182 bispecific antibody to human CD3 and human EpCAM at different pH values as tested by sandwich affinity ELISA. The mean OD values from 2 replicates for each pH tested are shown. Y-axis: OD₄₅₀ nm. X-axis: pH values.

FIG. 8 shows binding of BA3182 to human CD3 and human, cyno, rat and mouse EpCAM extra cellular domain at pH 6.0 and pH 7.4.

FIGS. 9A and 9B show binding of BA3182 to human CD3/human EpCAM and human CD3/human Trop2 extracellular domain, respectively, at pH 6.0 and pH 7.4. Binding of BA3182 to recombinant human CD3 and (A) human EpCAM or (B) human Trop2 ECD at pH 6.0 and pH 7.4 was measured by sandwich ELISA. Shown are the results of one representative experiment Black bars: BA3182 bispecific antibody; White bars: anti-Trop2 bispecific antibody.

FIGS. 10A and 10B show binding of BA3182 to human CD3/human EpCAM and human CD3/non-related human antigen extracellular domain, respectively, at pH 6.0 and pH 7.4. Binding of BA3182 to recombinant human CD3 and (A) human EpCAM or (B) non-related human antigen ECD at pH 6.0 and pH 7.4 was measured by sandwich ELISA. Shown are the results of one representative experiment. Black bars: BA3182 bispecific antibody; White bars: Positive control bispecific antibody targeting an undisclosed non-related antigen.

FIGS. 11A and 11B show binding of BA3182 to human EpCAM and human Trop2 extra cellular domain at pH 6.0 and pH 7.4. Binding of BA3182 to human EpCAM (FIG. 11A) and to human Trop2 ECD (FIG. 11B) at pH 6.0 and pH 7.4 was measured by affinity ELISA. Shown are the results of one representative experiment. Black bars: BA3182 bispecific antibody; Pattern bars: anti-Trop2 antibody.

FIGS. 12A, 12B and 12C show a representative gating strategy of FACS analysis of EpCAM positive cells. FACS analysis of CHO-hEpCAM cells stained with secondary antibody goat anti-hIgG₁ AF488 only. FIG. 12A shows density plots of a live cell population by forward scatter (FSC) versus side scatter (SSC). FIG. 12B shows a single cell population determined by forward scatter height (FSC-H) versus forward scatter area (FSC-A) under a live cells gate. FIG. 12C is a histogram plot showing the cutoff gate to determine AF488 positive single cells.

FIGS. 13A, 13B and 13C show binding analysis of BA3182 to EpCAM expressing cells at pH 6.0 and pH 7.4. Binding analysis of BA3182 to EpCAM antigen expressed on the cell surface is shown in FIG. 13A, to CHO hEpCAM cells and to CHO cynoEpCAM cells is shown in FIG. 13B and to HCT116 cells in FIG. 13C. Cells were stained with BA3182 at pH6.0 and pH7.4. Data shown is representative of three independent experiments. Y-axis: Median of fluorescence intensity (MFI) values. X-Axis: antibody concentration. The starting concentration of BA3182 for staining of CHO hEpCAM and HCT116 cells was 500 nM. For staining of CHO cynoEpCAM cells the initial concentration of BA3182 was 1500 nM.

FIGS. 14A, 14B and 14CC show binding analysis of BA3182 CD3 expressing cells at pH 6.0 and pH 7.4. Binding analysis of BA3182 to CD3 antigen expressed on the cells surface is shown for Human PBMC in FIG. 14A, for Cyno PBMC in FIG. 14B and for Jurkat cells in FIG. 14C. Cells were stained with BA3182 at pH 6.0 and pH 7.4. Y-axis: Median of fluorescence intensity (MFI) values. X-Axis: antibody concentration. The starting concentration of BA3182 for staining CD3 expressing cells was 2500 nM.

FIG. 15A shows the standard curve for the PE beads.

FIG. 15B shows expression levels of EpCAM antigen on CHO hEpCAM, CHO cynoEpCAM and HCT116 cell surfaces. The EPCAM expression levels on CHO-hEpCAM, CHO-cynoEpCAM, and HCT116 cells were estimated by QantiBrite™ PE quantitation kit from BD, which contain a mixture of beads that were loaded with known quantities of phycoerythrin (PE) molecules (high, medium, and low). The logarithmic geometric mean of PE fluorescence intensity from the beads, and the number of PE molecules per bead provided by the vendor were used to generate a standard curve. The number of PE molecules on EpCAM expressing cells, stained with an anti-hEpCAM PE conjugated antibody, was calculated by extrapolation from the bead standard curve using the logarithmic geometric mean of PE fluorescence on stained cells. The anti-hEpCAM antibody bound well to both human and cyno EpCAM expressing cells.

FIGS. 16A, 16B and 16C show in vitro functional activity of BA3182 at pH 6.0 and pH 7.4 against target cells expressing EpCAM. BA3182 mediated T cell activation at pH 6.0 and at pH7.4. (FIG. 16A) CHO hEpCAM cells. (FIG. 16B) CHO cynoEpCAM cells and (FIG. 16C) HCT116 cells. The mean RLU values from 2 replicates are shown. Y-axis: Relative Light Units (RLUs). X-axis: log antibody concentration (nM). Red circle: pH 6.0. Blue square: pH 7.4. Data is representative of three experiments.

FIGS. 17A, 17B and 17C show in vitro cytotoxicity activity of BA3182 at pH 6.5 and pH 7.4 against HCT116 with human PBMC and CHO cells expressing cynoEpCAM with cyno PBMC. Cytolysis of EpCAM expressing target cells mediated by PBMCs activated by BA3182 antibody at pH 6.5* and pH 7.4*. FIG. 17A shows HCT116 cells/human PBMC at pH 6.5, FIG. 17B shows HCT116 cells/human PBMC at pH 7.4 and FIG. 17C shows CHO cynoEpCAM/cyno PBMC. X-axis: Log concentrations of BA3182 in pM; Y-axis: Percentage of cytolysis. *N=10 at pH 6.5 and N=6 at pH 7.4, For 4 PBMC lots there were not enough cells for testing at both pH values.

FIGS. 18A and 18B show induction of IL-2 by stimulation of human PBMCs with BA3182 (FIG. 18A) and isotype control antibody (FIG. 18B) in the presence of HCT116 cancer cells. Cultures were maintained at 37° C. with 5% CO₂ for 48 h, supernatants were collected, and IL-2 cytokine was measured using the Human IL-2 Quantikine™ ELISA assay (R&D Systems). PBMCs from 9 human subjects were tested. Y-axis: IL-2 concentration in pg/mL. X-axis: log concentration of BA3182 or isotype control antibody.

FIGS. 19A and 19B show induction of INFγ by stimulation of human PBMCs with BA3182 (FIG. 19A) and isotype control antibody (FIG. 19B) in the presence of HCT116 cancer cells. Cultures were maintained at 37° C. with 5% CO₂ for 48 h, supernatants were collected, and IL-2 cytokine was measured using the Human IFNγ Quantikine™ ELISA assay (R&D Systems). PBMCs from 9 human subjects were tested. Y-axis: IFNγ concentration in pg/mL. X-axis: log concentration of BA3182 or isotype control antibody.

FIGS. 20A and 20B show induction of IL-6 by stimulation of human PBMCs with BA3182 (FIG. 20A) and isotype control antibody (FIG. 20B) in the presence of HCT116 cancer cells. Cultures were maintained at 37° C. with 5% CO₂ for 48 h, supernatants were collected, and IL-2 cytokine was measured using the Human IL-6 Quantikine™ ELISA assay (R&D Systems). PBMCs from 9 human subjects were tested. Y-axis: IL-6 concentration in pg/mL. X-axis: log concentration of BA3182 or isotype control antibody.

FIGS. 21A and 21B show induction of IL-10 by stimulation of human PBMCs with BA3182 (FIG. 21A) and isotype control antibody (FIG. 21B) in the presence of HCT116 cancer cells. Cultures were maintained at 37° C. with 5% CO₂ for 48 h, supernatants were collected, and IL-2 cytokine was measured using the Human IL-10 Quantikine™ ELISA assay (R&D Systems). PBMCs from 9 human subjects were tested. Y-axis: IL-10 concentration in pg/mL. X-axis: log concentration of BA3182 or isotype control antibody.

FIGS. 22A and 22B show induction of TNFα by stimulation of human PBMCs with BA3182 and isotype control antibody in the presence of HCT116 cancer cells. Cultures were maintained at 37° C. with 5% CO₂ for 48 h, supernatants were collected, and IL-2 cytokine was measured using the Human TNFα Quantikine™ ELISA assay (R&D Systems). PBMCs from 9 human subjects were tested. Y-axis: TNFα concentration in pg/mL. X-axis: log concentration of BA3182 or isotype control antibody.

FIG. 23 shows an ELISA assay demonstrating binding of human C1q protein to BA3182 and B12 antibodies. B12, a human IgG_{1.k} antibody against HIV viral envelope protein 120 (gp120), was used as a positive control for this assay. The mean OD values from 2 replicates are shown. Data is representative of two experiments. Y-axis: OD 450 nm. X-axis: Concentration of C1q in nM.

FIG. 24 shows SPR Sensorgrams of BA3182 binding to human and cyno EpCAM extracellular domain at different pH values. Binding of BA3182 to human and cynomolgus EpCAM was measured by SPR at pH 6.0, pH 6.5, and pH 7.4. Shown are the binding curves of one representative experiment at each pH value. Data were fitted with a 1:1 Langmuir binding model. The maximum binding signal drops from pH 6.0 to pH 7.4 because of the engineered pH-dependent binding of BA3182. Left column: Sensorgrams of BA3182 binding to human EpCAM at pH 6.0 (top), pH 6.5 (middle), and pH 7.4 (bottom). Right column: Sensorgrams of BA3182 binding to cynomolgus EpCAM at pH 6.0 (top), pH 6.5 (middle), and pH 7.4 (bottom).

FIG. 25 shows SPR Sensorgrams of BA3182 binding to human and cynoCD3 extracellular domain at different pH values. Binding of BA3182 to human and cynomolgus CD3 was measured by SPR at pH 6.0, pH 6.5, and pH 7.4. Shown are the binding curves of one representative experiment at each pH value. Data were fitted with a 1:1 Longmuir binding model. The maximum binding signal drops from pH 6.0 to pH 7.4 because of the engineered pH-dependent binding of BA3182. Left column: Sensorgrams of BA3182 binding to human CD3 at pH 6.0 (top), pH 6.5 (middle), and pH 7.4 (bottom). Right column: Sensorgrams of BA3182 binding to cynomolgus CD3 at pH 6.0 (top), pH 6.5 (middle), and pH 7.4 (bottom).

FIG. 26 shows analysis of BA3182 binding kinetics to human EpCAM at different pH conditions using SPR simulation software. Simulated sensorgrams (on the right side) were generated using the SPR-simulation software as described in 3.6. The simulation shows that the drop in maximum signal from pH 6.0 (top) to pH 6.5 (middle), and pH 7.4 (bottom) can best be simulated by a reduction of active ligand on the sensor surface.

FIG. 27 shows analysis of BA3182 binding kinetics to human CD3 at different pH values using SPR simulation software. Simulated sensorgrams (on the right side) were generated using the SPR-simulation software as described in 3.6. The simulation shows that the drop in maximum signal from pH 6.0 (top) to pH 6.5 (middle), and pH 7.4 (bottom) can best be simulated by a reduction of active ligand on the sensor surface.

FIG. 28 shows BA3182 binding to FcγRI (CD64). Sensorgrams of BA3182 (top left) and IgG1-ctrl mAb (top right) are shown at the same scale. Bottom: Kinetic fitting of the BA3182 binding to FcγRI with a steady state model. The sensorgrams for reference spots and buffer only injections were subtracted from the test sensorgrams. The experimental data for the IgG1-ctrl mAb were fitted with a 1:1 binding model, and data for BA3182 were fitted with a steady state model (blue: IgG1-Ctrl mAb, red: BA3182). Molecular weights of 150 kDa for the IgG1-ctrl mAb and 200 kDa for BA3182 were used to calculate the molar concentration. The IgG1-ctrl antibody with a wild-type human IgG1 Fc domain binds to FcγRI with an affinity of 1.18 nM. The binding signal for BA3182 was much lower and signal was detected only with the three highest concentrations tested. All binding curves reach saturation, so a steady state model was used to analyze the data. However, the signal intensity and differences between the different concentrations are too low to calculate a meaningful K_{D}. The data indicate that BA3182 does not bind to FcγRI.

FIG. 29 shows BA3182 binding to FcγRIIa (CD32a). Sensorgrams of BA3182 (top left) and IgG1-ctrl mAb (top right) are shown at the same scale. Bottom: Kinetic fitting of the experimental data to FcγRIIa with a steady state model (blue: IgG1-Ctrl mAb, red: BA3182). The sensorgrams for reference spots and buffer only injections were subtracted from the test sensorgrams. Molecular weights of 150 kDa for the IgG1-ctrl mAb and 200 kDa for the BA3182 were used to calculate the molar concentration. The IgG1-ctrl antibody with a wild-type human IgG1 Fc domain binds to FcγRIIa with an affinity of 0.82 μM. No binding to FcγRIIa was detected for BA3182 indicating that BA3182 does not interact with FcγRIIa.

FIG. 30 shows BA3182 binding to FcγRIIb/c (CD32b/c). Sensorgrams of BA3182 (top left) and IgG1-ctrl mAb (top right) are shown at the same scale. Bottom: Kinetic fitting of the experimental data to FcγRIIb/c with a steady state model (blue: IgG1-Ctrl mAb, red: BA3182). The sensorgrams for reference spots and buffer only injections were subtracted from the test sensorgrams. Molecular weights of 150 kDa for the IgG1-ctrl mAb and 200 kDa for the BA3182 were used to calculate the molar concentration. The IgG1-ctrl antibody with a wild-type human IgG1 Fc domain binds to FcγRIIb/c with an affinity of 4.2 μM. No binding to FcγRIIb/c was detected for BA3182 indicating that BA3182 does not interact with FcγRIIb/c.

FIG. 31 shows BA3182 binding to FcγIIIa CD16a (F158). Sensorgrams of BA3182 (top left) and IgG1-ctrl mAb (top right) are shown at the same scale. Bottom: Kinetic fitting of the experimental data to FcγRIIIa with a steady state model (blue: IgG1-Ctrl mAb, red: BA3182). The sensorgrams for reference spots and buffer only injections were subtracted from the test sensorgrams. Molecular weights of 150 kDa for the IgG1-ctrl mAb and 200 kDa for BA3182 were used to calculate the molar concentration. The IgG1-ctrl antibody with a wild-type human IgG1 Fc domain binds to FcγRIIIa with an affinity of 0.86 μM. No binding to FcγRIIIa was detected for BA3182 indicating that BA3182 does not interact with FcγRIIIa.

FIG. 32 shows BA3182 binding to FcγIIb (CD16b). Sensorgrams of BA3182 (top left) and IgG1-ctrl mAb (top right) are shown at the same scale. Bottom: Kinetic fitting of the experimental data to FcγRIIIb with a steady state model (blue: IgG1-Ctrl mAb, red: BA3182). The sensorgrams for reference spots and buffer only injections were subtracted from the test sensorgrams. Molecular weights of 150 kDa for the IgG1-ctrl mAb and 200 kDa for the BA3182 were used to calculate the molar concentration. The IgG1-ctrl antibody with a wild-type human IgG1 Fc domain binds to FcγRIIIa with an affinity of 6.5 μM. No binding to FcγRIIIa was detected for BA3182 indicating that BA3182 does not interact with FcγRIIIa.

FIG. 33 shows BA3182 binding to FcRn. Binding of BA3182 to FcRn. Top: Sensorgrams of BA3182 (left) and IgG1-ctrl mAb (right) are shown at the same scale. Bottom: Kinetic fitting of the experimental data to FcRn with a steady state model (blue:BA3182 red: IgG1-Ctrl mAb). The sensorgrams for reference spots and buffer only injections were subtracted from the test sensorgrams. Molecular weight of 61.91 kDa was used to calculate the molar concentration of the FcRn. The IgG1-Ctrl antibody with a wild-type human IgG1 Fc domain binds to FcRn with an affinity of 366 nM. Similarly, BA3182 showed a binding affinity of 347 nM to FcRn.

FIGS. 34A-34H show cytokine release in culture of human PBMCs stimulated with soluble BA3182 or CD3/CD28 dynabeads at pH 6.5. Cultures of human PBMCs were stimulated with soluble BA3182 or CD3/CD28 dynabeads for 48 hrs in culture media at pH 6.5. Supernatants were collected and the concentrations of cytokines were measured using a Luminex Multiplex Cytokine Assay. Data shows the levels of various cytokines in pg/mL in cultures of human PBMC from 9 different donors, IFNγ (FIG. 34A), IL1ß (FIG. 34B), IL2 (FIG. 34C), IL4 (FIG. 34D), IL6 (FIG. 34E), IL10 (FIG. 34F), IL17 (FIG. 34G) and TNFα (FIG. 34H), with CD3/CD28 dynabeads, but none were detected from soluble BA3182 treatment.

FIGS. 35A-35H show cytokine release in culture of human PBMCs stimulated with soluble BA3182 or CD3/CD28 dynabeads at pH 7.4. Cultures of human PBMCs were stimulated with CD3/CD28 dynabeads for 48 hrs in culture media at pH 7.4. Supernatants were collected and the concentrations of cytokines were measured using a Luminex Multiplex Cytokine Assay. Data shows the levels of various cytokines in pg/mL in cultures of human PBMC from 9 different donors IFNγ (FIG. 35A), IL1ß (FIG. 35B), IL2 (FIG. 35C), IL4 (FIG. 35D), IL6 (FIG. 35E), IL10 (FIG. 35F), IL17 (FIG. 35G) and TNFα (FIG. 35H), with CD3/CD28 dynabeads, but none were detected from soluble BA3182 treatment.

FIGS. 36A-36H show cytokine production in cultures of human PBMCs stimulated with immobilized BA3182 and anti-CD3 antibody, clone OKT3 at pH 6.5. Cultures of human PBMCs were stimulated with immobilized BA3182 or OKT3 for 48 hrs in culture media at pH 6.5. Supernatants were collected and the concentrations of cytokines were measured using a Luminex Multiplex Cytokine Assay. Data shows the levels of various cytokines in pg/mL in cultures of human PBMC from 9 donors, IFNγ (FIG. 36A), IL1ß (FIG. 36B), IL2 (FIG. 36C), IL4 (FIG. 36D), IL6 (FIG. 36E), IL10 (FIG. 36F), IL17 (FIG. 36G) and TNFα (FIG. 36H).

FIGS. 37A-37H show cytokine production in cultures of human PBMCs stimulated with immobilized BA3182 and anti-CD3 antibody, clone OKT3 at pH 7.4. Cultures of human PBMCs were stimulated with immobilized BA3182 or OKT3 for 48 hrs in culture media at pH 7.4. Supernatants were collected and the concentrations of cytokines were measured using a Luminex Multiplex Cytokine Assay. Data shows the levels of various cytokines in pg/mL in cultures of human PBMC from 9 donors, IFNγ (FIG. 37A), IL1ß (FIG. 37B), IL2 (FIG. 37C), IL4 (FIG. 37D), IL6 (FIG. 37E), IL10 (FIG. 37F), IL17 (FIG. 37G) and TNFα (FIG. 37H).

FIG. 38 shows the BAP150.31-BF45 dose-response binding curve of BAP150.31-BF45 to human EpCAM antigen measured by affinity ELISA. Average of OD values from 2 replicates were plotted against different concentrations of BAP150.31-BF45. X-axis: Log concentrations of BAP150.31-BF45 in ng/mL, Y-axis: OD values at 450 nm.

FIG. 39 shows the serum concentration-time profiles following a single 1 mg/kg IV administration of BAP150.31-BF45 in mice. The average of serum concentrations of BAP150.31-BF45 following a single 1 mg/kg IV administration of BAP150.31-BF45 were plotted against different serum sampling time points using PK Solver 2.0.

FIG. 40 shows the serum concentration-time profiles of BAP150.31-BF45 following a single 10 mg/kg IV administration of BAP150.31-BF45 in mice. The average of serum concentrations of BAP150.31-BF45 following a single 10 mg/kg IV administration of BAP150.31-BF45 were plotted against different sampling time points using PK Solver 2.0.

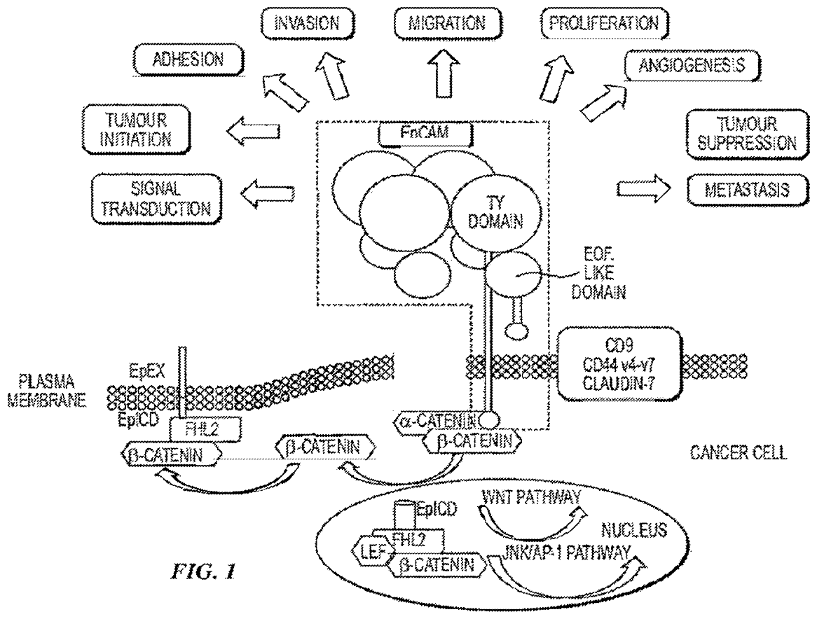

### DEFINITIONS

In order to facilitate understanding of the examples provided herein, certain frequently occurring terms are defined herein.

In connection with a measured quantity, the term “about” as used herein refers to the normal variation in that measured quantity that would be expected by a skilled person making the measurement and exercising a level of care commensurate with the objective of the measurement and the precision of the measuring equipment used. Unless otherwise indicated, “about” refers to a variation of +/−10% of the value provided.

The term “aberrant condition” as used herein refers to a condition that deviates from the normally acceptable range in a subject for that condition. The term “normal physiological condition” as used herein refers to a condition that is considered within a normal range at a location in a subject such as at the site of administration, or at the tissue or organ at the site of action, in a subject.

The term “affinity” as used herein refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described herein.

The term “affinity matured” antibody as used herein refers to an antibody with one or more alterations in one or more heavy chain or light chain variable regions, compared to a parent antibody which does not possess such alterations, such alterations resulting in an improvement in the affinity of the antibody for antigen.

The term “amino acid” as used herein refers to any organic compound that contains an amino group (—NH2) and a carboxyl group (—COOH); preferably either as free groups or alternatively after condensation as part of peptide bonds. The “twenty naturally encoded polypeptide-forming alpha-amino acids” are understood in the art and refer to: alanine (ala or A), arginine (arg or R), asparagine (asn or N), aspartic acid (asp or D), cysteine (cys or C), gluatamic acid (glu or E), glutamine (gin or Q), glycine (gly or G), histidine (his or H), isoleucine (ile or I), leucine (leu or L), lysine (lys or K), methionine (met or M), phenylalanine (phe or F), proline (pro or P), serine (ser or S), threonine (thr or T), tryptophan (tip or W), tyrosine (tyr or Y), and valine (val or V).

The term “antibody” as used herein refers to intact immunoglobulin molecules, as well as fragments of immunoglobulin molecules, such as Fab, Fab′, (Fab′)2, Fv, and SCA fragments, that are capable of binding to an epitope of an antigen. These antibody fragments, which retain some ability to selectively bind to an antigen (e.g., a polypeptide antigen) of the antibody from which they are derived, can be made using well known methods in the art such as those described herein. Unless otherwise stated, the use of the term antibody or antibodies includes functional fragments of the antibody or antibodies. Antibodies can be used to isolate preparative quantities of the antigen by immunoaffinity chromatography. Various other uses of such antibodies are to diagnose and/or stage disease (e.g., neoplasia) and for therapeutic application to treat disease, such as for example: neoplasia, autoimmune disease, AIDS, cardiovascular disease, infections, and the like. Chimeric, human-like, humanized or fully human antibodies are particularly useful for administration to human patients. Antibodies and antibody fragments of the present disclosure can be obtained by evolving or mutation of a parent antibody or antibody fragment that has the same type of activity, e.g. binding activity or affinity to EpCAM protein.

An Fab fragment consists of a monovalent antigen-binding fragment of an antibody molecule, and can be produced by digestion of a whole antibody molecule with the enzyme papain, to yield a fragment consisting of an intact light chain and a portion of a heavy chain.

An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner.

An (Fab′)2 fragment of an antibody can be obtained by treating a whole antibody molecule with the enzyme pepsin, without subsequent reduction. A (Fab′)2 fragment is a dimer of two Fab′ fragments, held together by two disulfide bonds.

An Fv fragment is defined as a genetically engineered fragment containing the variable region of a light chain and the variable region of a heavy chain expressed as two chains.

The term “antibody fragment” as used herein refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The terms “anti-EpCAM antibody,” “EpCAM antibody” and “an antibody that binds to EpCAM” as used herein refer to an antibody that is capable of binding to an EpCAM protein or an epitope of an EpCAM protein with sufficient affinity such that the antibody is useful as a diagnostic and/or therapeutic agent in targeting EpCAM. In one embodiment, the extent of binding of an anti-EpCAM antibody to an unrelated, non-EpCAM protein is less than about 10% of the binding of the antibody to EpCAM as measured, e.g., by a radioimmunoassay (RIA). In certain embodiments, an antibody that binds to EpCAM has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In certain embodiments, an anti-EpCAM antibody binds to an epitope of EpCAM that is conserved among EpCAM from different species, for example, the extracellular domain of EpCAM.

The term “antigen” or “Ag” as used herein is defined as a molecule that provokes an immune response. This immune response may involve either antibody production, or the activation of specific immunologically-competent cells, or both. A person skilled in the art will understand that any macromolecule, including virtually all proteins or peptides, and polysaccharides, nucleic acids or lipids, can serve as an antigen. Furthermore, antigens can be derived from recombinant or genomic DNA. A person skilled in the art will understand that any DNA, which includes a nucleotide sequence or a partial nucleotide sequence encoding a protein that elicits an immune response therefore encodes an “antigen” as that term is used herein. Furthermore, a person skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled person will understand that an antigen need not be encoded by a “gene” at all. It is readily apparent that an antigen can be generated, synthesized or can be derived from a biological sample. Such a biological sample can include, but is not limited to, a tissue sample, a tumor sample, a cell or a biological fluid.

The term “binding” as used herein refers to interaction of the variable region or an Fv of an antibody with an antigen with the interaction depending upon the presence of a particular structure (e.g., an antigenic determinant or epitope) on the antigen. For example, an antibody variable region or Fv recognizes and binds to a specific protein structure rather than to proteins generally. As used herein, the term “specifically binding” or “binding specifically” means that an antibody variable region or Fv binds to or associates with more frequently, more rapidly, with greater duration and/or with greater affinity with a particular antigen than with other proteins. For example, an antibody variable region or Fv specifically binds to its antigen with greater affinity, avidity, more readily, and/or with greater duration than it binds to other antigens. For another example, an antibody variable region or Fv binds to a cell surface protein (antigen) with materially greater affinity than it does to related proteins or other cell surface proteins or to antigens commonly recognized by polyreactive natural antibodies (i.e., by naturally occurring antibodies known to bind a variety of antigens naturally found in humans). However, “specifically binding” does not necessarily require exclusive binding or non-detectable binding of another antigen, this is meant by the term “selective binding”. In one example, “specific binding” of an antibody variable region or Fv (or other binding region) binds to an antigen, means that the an antibody variable region or Fv binds to the antigen with an equilibrium constant (KD) of 100 nM or less, such as 50 nM or less, for example 20 nM or less, such as, 15 nM or less, or 10 nM or less, or 5 nM or less, 2 nM or less, or 1 nM or less.

The terms “cancer” and “cancerous” as used herein refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

The term “cell antigen” or “cell associated antigen” as used herein refers to any protein, carbohydrate or other component derived from or expressed by a cell which is capable of eliciting an immune response. For example, the cell may be any cell in the subject, particularly a cancer cell and senescent cell. The cell antigen may be an antigen on the surface of the cell or inside of the cell. The definition is meant to include, but is not limited to, proteins purified from the cell surface or membrane of a cell, or unique carbohydrate moieties associated with the cell surface of a cell. The definition also includes those antigens from the surface of the cell which require special treatment of the cells to be accessed by an antibody of the present disclosure.

The terms “cell proliferative disorder” and “proliferative disorder” as used herein refer to disorders that are associated with some degree of abnormal cell proliferation. In one embodiment, the cell proliferative disorder is cancer.

The term “chimeric” antibody as used herein refers to an antibody in which a portion of the heavy and/or light chain is derived from a particular source or species, while the remainder of the heavy and/or light chain is derived from a different source or species.

The term “conditionally active antibody” as used herein refers to an anti-EpCAM antibody or antibody fragment which is more active under a condition in the tumor microenvironment compared to under a condition in the non-tumor microenvironment. The conditions in the tumor microenvironment include lower pH, higher concentrations of lactate and pyruvate, hypoxia, lower concentration of glucose, and slightly higher temperature in comparison with non-tumor microenvironment. For example, a conditionally active antibody is virtually inactive at normal body temperature but is active at a higher temperature in a tumor microenvironment. In yet another aspect, the conditionally active antibody is less active in normal oxygenated blood, but more active under a less oxygenated environment exists in tumor. In yet another aspect, the conditionally active antibody is less active in normal physiological of pH 7.0-7.6 or 7.2-7.6, but more active under an acidic pH 5.0-6.9, or 6.0-6.8 that exists in a tumor microenvironment. There are other conditions in the tumor microenvironment know to a person skilled in the field may also be used as the condition in the present disclosure under which the anti-EpCAM antibodies to have different binding affinity to EpCAM.

The term “diabodies” as used herein refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementarity domains of another chain and create two antigen-binding sites.

The term “detectably label” as used herein refers to any substance whose detection or measurement, either directly or indirectly, by physical or chemical means, is indicative of the presence of an antigen in a sample. Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecules.

The term “diagnostics” as used herein refers to determination of a subject's susceptibility to a disease or disorder, determination as to whether a subject is presently affected by a disease or disorder, prognosis of a subject affected by a disease or disorder (e. g., identification of pre-metastatic or metastatic cancerous states, stages of cancer, or responsiveness of cancer to therapy), and therametrics (e. g., monitoring a subject's condition to provide information as to the effect or efficacy of therapy). In some embodiments, the diagnostic method of this disclosure is particularly useful in detecting early stage cancers.

The term “effector functions” as used herein refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “effective amount” of an agent as used herein, e.g., a pharmaceutical formulation, refers to an amount effective, at dosages and for periods of time necessary, to achieve the desired therapeutic or prophylactic result.

The term “epitope” or “antigenic determinant” as used herein refers to a site on an antigen to which an antibody binds. Epitopes can be formed both from contiguous amino acids (linear epitope) or noncontiguous amino acids juxtaposed by tertiary folding of a protein (conformational epitopes). Epitopes formed from contiguous amino acids are typically retained on exposure to denaturing solvents whereas epitopes formed by tertiary folding are typically lost on treatment with denaturing solvents. An epitope can comprise 3 or more amino acids. Usually an epitope consists of at least 5 to 7 amino acids (such as 5, 6, or 7 amino acids in an epitope), or of at least 8-11 amino acids (such as 8, 9, 10 or 11 amino acids in an epitope), or of more than 11 amino acids (such as 12, 13, 14, 15, 16, 17, 18, 19 or 20 amino acids in an epitope), or of more than 20 amino acids (such as 21, 22, 23, 24, 25, 26, 27, 28, 29, or 30 amino acids in an epitope), and, less frequently, an epitope can contain 31-40 amino acids. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). A preferred method for epitope mapping on an antigen is surface plasmon resonance.

The term “Fc region” as used herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The term “framework” or “FR” as used herein refers to variable domain residues other than complementarity determining regions (CDRs or H1-3 in the heavy chain and L1-3 in the light chain) residues. The FR of a variable domain generally consists of four FR domains: FR1, FR2, FR3, and FR4. Accordingly, the CDR and FR sequences generally appear in the following sequence in V_{H} (or V_{L}): FR1-H1(L1)-FR2-H2(L2)-FR3-H3(L3)-FR4.

The term “full length antibody,” “intact antibody,” or “whole antibody” refers to an antibody which comprises an antigen-binding variable region (V_{H} or V_{L}) as well as a light chain constant domain (CL) and heavy chain constant domains, CH1, CH2 and CH3. The constant domains may be native sequence constant domains (e.g. human native sequence constant domains) or amino acid sequence variants thereof. Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

The term “human antibody” as used herein is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “humanized” antibody as used herein refers to a chimeric antibody comprising amino acid residues from non-human CDRs and amino acid residues from human FRs. In certain embodiments, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “individual,” “patient” or “subject” is a human or an animal. For example, the subject may be a mammal selected from domesticated animals (e.g., cows, sheep, cats, dogs, and horses), primates (e.g., humans and non-human primates such as monkeys), rabbits, and rodents (e.g., mice and rats).

The term “isolated” antibody as used herein is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase High Performance Liquid Chromatography (HPLC)). For review of methods for assessment of antibody purity, see, e.g., Flatman et al., J. Chromatogr. B, vol. 848, pp. 79-87, 2007.

The term “metastasis” as used herein refers to all EpCAM-involving processes that support cancer cells to disperse from a primary tumor, penetrate into lymphatic and/or blood vessels, circulate through the bloodstream, and grow in a distant focus (metastasis) in normal tissues elsewhere in the body. In particular, it refers to cellular events of tumor cells such as proliferation, migration, anchorage independence, evasion of apoptosis, or secretion of angiogenic factors, that underlie metastasis and are stimulated or mediated by EpCAM.

The term “microenvironment” as used herein means any portion or region of a tissue or body that has constant or temporal, physical or chemical differences from other regions of the tissue or regions of the body. For tumors, the term “tumor microenvironment” as used herein refers to the environment in which a tumor exists, which is the non-cellular area within the tumor and the area directly outside the tumorous tissue but does not pertain to the intracellular compartment of the cancer cell itself. The tumor and the tumor microenvironment are closely related and interact constantly. A tumor can change its microenvironment, and the microenvironment can affect how a tumor grows and spreads. Typically, the tumor microenvironment has a low pH in the range of 5.0 up to 7.0, or in the range of 5.0 to 6.9, or in the range of 5.8 to 6.8, or in the range of 6.2-6.8. On the other hand, a normal physiological pH is in the range of 7.0 to 7.6 or 7.2-7.8. The tumor microenvironment is also known to have lower concentration of glucose and other nutrients, but higher concentration of lactic acid, in comparison with blood plasma. Furthermore, the tumor microenvironment can have a temperature that is 0.3 to 1° C. higher than the normal physiological temperature. The tumor microenvironment has been discussed in Gillies et al., “MRI of the Tumor Microenvironment,” Journal of Magnetic Resonance Imaging, vol. 16, pp. 430-450, 2002, hereby incorporated by reference herein its entirety. The term “non-tumor microenvironment” refers to a microenvironment at a site other than a tumor.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci.

The term “multi-specific antibody” as used herein is a full-length antibody, an antibody fragment or a construct comprising one or more full-length antibodies and antibody fragments, which has at least two different binding sites each capable of binding to an epitope on the same or different antigen. Constructs of engineered antibodies with two, three or more (e.g. four, five, six, or seven) functional antigen binding sites are within the scope of the multi-specific antibody (see, e.g., US 2002/0004587 A1 and Brinkman and Kontermann, MAbs, vol. 9, pp. 182-212, 2017).

The term “package insert” as used herein is used to refer to instructions customarily included in commercial packages of therapeutic products, that contain information about the indications, usage, dosage, administration, combination therapy, contraindications and/or warnings concerning the use of such therapeutic products.

The term “percent (%) amino acid sequence identity” with respect to a reference polypeptide sequence as used herein is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term “pharmaceutical formulation” as used herein refers to a preparation which is in such form as to permit the biological activity of an active ingredient contained therein to be effective, and which contains no additional components which are unacceptably toxic to a subject to which the formulation would be administered.

The term “pharmaceutically acceptable carrier” as used herein refers to an ingredient in a pharmaceutical formulation, other than an active ingredient, which is nontoxic to a subject. A pharmaceutically acceptable carrier includes, but is not limited to, a buffer, excipient, stabilizer, or preservative.

The terms “purified” and “isolated” used herein refer to an antibody according to the disclosure or to a nucleotide sequence, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated” nucleic acid molecule which encodes a particular polypeptide refers to a nucleic acid molecule which is substantially free of other nucleic acid molecules that do not encode the polypeptide; however, the molecule may include some additional bases or moieties which do not deleteriously affect the basic characteristics of the composition.

The term “recombinant antibody” as used herein refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include: (1) mammalian cells, for example, Chinese Hamster Ovary (CHO), COS, myeloma cells (including Y0 and NS0 cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia. coli cells or Bacillus subtilis cells, etc.

The term “single chain Fv” (“scFv”) as used herein is a covalently linked V_{H}::V_{L} heterodimer which is usually expressed from a gene fusion including V_{H} and V_{L} encoding genes linked by a peptide-encoding linker. “dsFv” is a V_{H}::V_{L} heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

The term “therapeutically effective amount” of the antibody of the disclosure is meant a sufficient amount of the antibody to treat said cancer, at a reasonable benefit/risk ratio applicable to any medical treatment. It will be understood, however, that the total daily usage of the antibodies and compositions of the present disclosure will be decided by the attending physician within the scope of sound medical judgment. The specific therapeutically effective dose level for any particular patient will depend upon a variety of factors including the disorder being treated and the severity of the disorder; activity of the specific antibody employed; the specific composition employed, the age, body weight, general health, sex and diet of the patient; the time of administration, route of administration, and rate of excretion of the specific antibody employed; the duration of the treatment; drugs used in combination or coincidental with the specific antibody employed; and like factors well known in the medical arts. For example, it is well known within the skill of the art to start doses of the compound at levels lower than those required to achieve the desired therapeutic effect and to gradually increase the dosage until the desired effect is achieved.

The term “treatment,” “treat,” or “treating” as used herein refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the disclosure are used to delay development of a disease or to slow the progression of a disease.

The term “tumor” as used herein refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The term “variable region” or “variable domain” as used herein refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three complementarity determining regions (CDRs). (See, e.g., Kindt et al. Kuby Immunology, 6th ed., W.H. Freeman and Co., page 91 (2007).) A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementarity V_{L} or V_{H} domains, respectively. See, e.g., Portolano et al., J. Immunol., vol. 150, pp. 880-887, 1993; Clarkson et al., Nature, vol. 352, pp. 624-628, 1991.

The term “unit dosage form” as used herein refers to physically discrete units suitable as unitary dosages for a subject, each unit containing a predetermined quantity of conditionally active multi-specific antibody of the present disclosure calculated in an amount of the multi-specific antibody of the present disclosure sufficient to produce the desired therapeutic effect in association with a pharmaceutically acceptable diluent, carrier or vehicle.

### DETAILED DESCRIPTION

It must be noted that as used herein and in the appended claims, the singular forms “a”, “an”, and “the” include plural references unless the context clearly dictates otherwise. Furthermore, the terms “a” (or “an”), “one or more,” and “at least one” can be used interchangeably herein. The terms “comprising,” “including,” “having,” and “constructed from” can also be used interchangeably.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

It is to be understood that each component, compound, substituent or parameter disclosed herein is to be interpreted as being disclosed for use alone or in combination with one or more of each and every other component, compound, substituent or parameter disclosed herein.

It is also to be understood that each amount/value or range of amounts/values for each component, compound, substituent or parameter disclosed herein is to be interpreted as also being disclosed in combination with each amount/value or range of amounts/values disclosed for any other component(s), compounds(s), substituent(s) or parameter(s) disclosed herein and that any combination of amounts/values or ranges of amounts/values for two or more component(s), compounds(s), substituent(s) or parameters disclosed herein are thus also disclosed in combination with each other for the purposes of this description.

It is further understood that each range disclosed herein is to be interpreted as a disclosure of each specific value within the disclosed range that has the same number of significant digits. Thus, a range of from 1-4 is to be interpreted as an express disclosure of the values 1, 2, 3 and 4. It is further understood that each lower limit of each range disclosed herein is to be interpreted as disclosed in combination with each upper limit of each range and each specific value within each range disclosed herein for the same component, compound, substituent or parameter. Thus, this disclosure is to be interpreted as a disclosure of all ranges derived by combining each lower limit of each range with each upper limit of each range or with each specific value within each range, and by combining each upper limit of each range with each specific value within each range.

Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter.

In one aspect, the present disclosure provides an isolated antibody that specifically binds to an EpCAM protein, in particular, a human EpCAM protein, comprising at least one heavy chain variable region containing three complementarity determining regions, H1, H2 and H3, and at least one light chain variable region containing three complementarity determining regions, L1, L2 and L3. Specific examples of suitable combinations of heavy chain and light chain complementarity determining regions are provided in Table 1.

Also provided are exemplary combinations of light and heavy chain variable regions. These include antibodies comprising the heavy chain variable region of SEQ ID NO: 52 in combination with any one of light chain variable regions of SEQ ID NOs: 53-69 or the light chain variable region of SEQ ID NO: 51 in combination with any one of the light chain variable regions of SEQ ID NOs: 70-96.

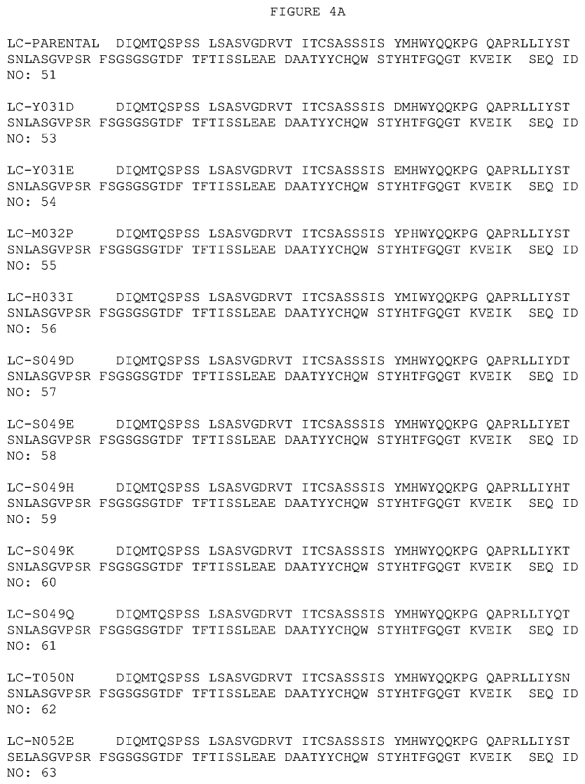

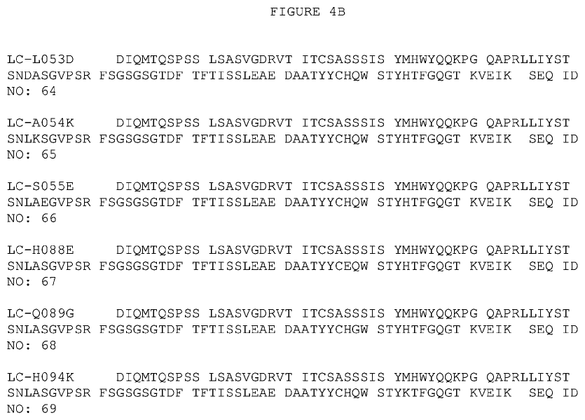

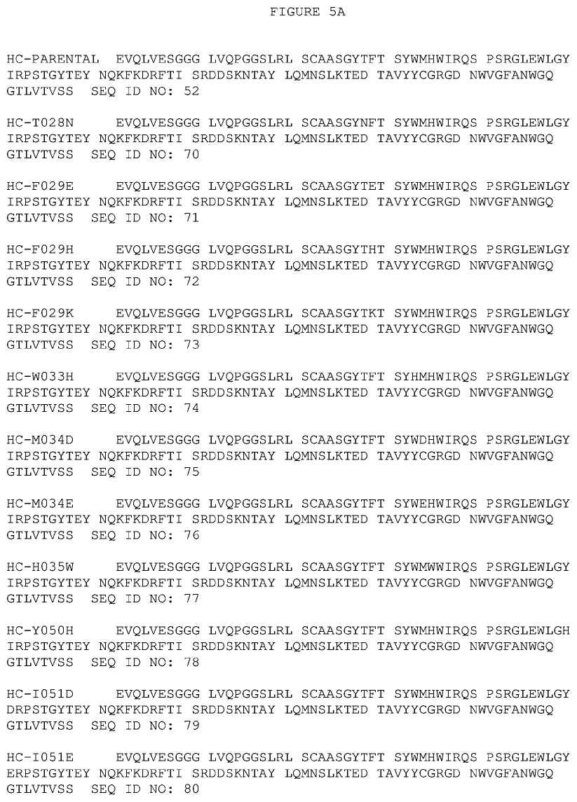

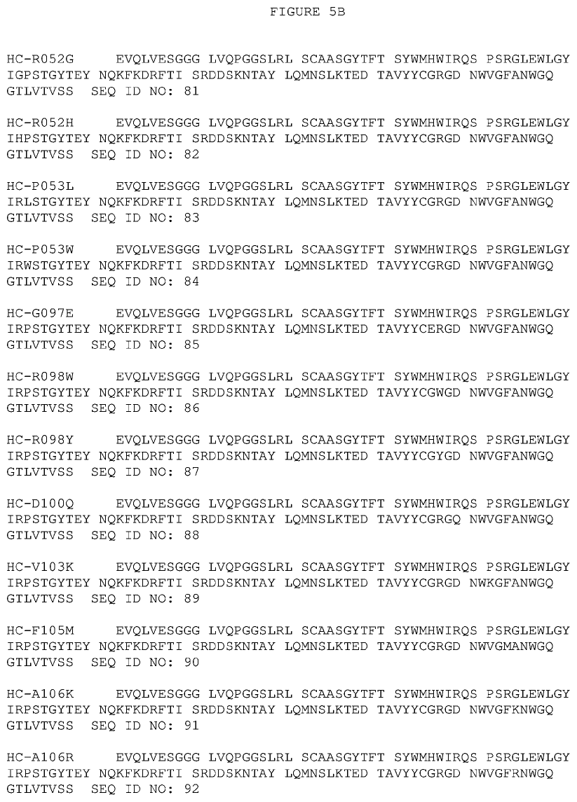

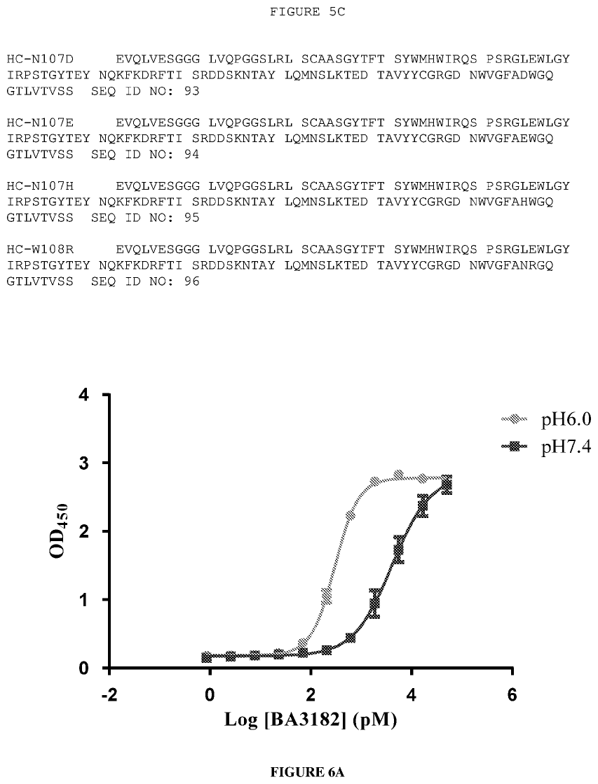

Alignments of the various embodiments of the light chain variable regions can be found in FIGS. 4A-4B and alignments of the various embodiments of the heavy chain variable regions can be found in FIGS. 5A-5B

The heavy chain variable regions and the light chain variable regions of the present disclosure were each obtained from a parental (wild-type) antibody using a method disclosed in U.S. Pat. No. 8,709,755. This method of generating the heavy chain variable regions and the light chain variable regions, as well as the method of generating antibodies and antibody fragments disclosed in U.S. Pat. No. 8,709,755, are hereby incorporated by reference herein.

Antibodies and antibody fragments including these heavy chain variable regions and light chain variable regions can specifically bind to EpCAM, for example, human EpCAM. Antibodies or antibody fragments comprising a combination of one of these heavy chain variable regions and one of these light chain variable regions have been found to have higher binding affinity to EpCAM at a pH in the tumor microenvironment (e.g. pH 6.0-6.8) than at a pH in a non-tumor microenvironment (e.g. pH 7.0-7.6). As a result, the anti-EpCAM antibodies or antibody fragments have a higher binding affinity to EpCAM in a tumor microenvironment in comparison with their binding affinity to EpCAM in a typical normal tissue microenvironment.

In another embodiment, the present disclosure provides a multi-specific antibody comprising at least one binding site for EpCAM and at least one binding site for a tumor-reactive lymphocyte antigen. The multi-specific antibody binds to at least one EpCAM epitope and the tumor-reactive lymphocyte antigen with a greater affinity at a first physiological condition than at a second physiological condition. In one particular embodiment, the tumor-reactive lymphocyte antigen is CD3. In some embodiments, the first physiological condition is an aberrant condition, and the second physiological condition is a normal physiological condition. For example, the aberrant condition may be a condition in a tumor microenvironment. The multi-specific antibody of the present disclosure may be referred to as a conditionally active multi-specific antibody.

In some embodiments, the conditionally active multi-specific antibody is virtually inactive at a normal physiological condition but is active at an aberrant condition, optionally having a level of activity that is higher than the activity of the conditionally active multi-specific antibody at a normal physiological condition or the activity at a normal physiological condition of the parent antibody from which it is derived. In another embodiment, the conditionally active multi-specific antibody is virtually inactive at a pH of 7.0-7.6 but is active at a lower pH of 5.0-6.8. In some cases, the conditionally active multi-specific antibody is reversibly inactivated at the normal physiological condition. In another example, the conditionally active multi-specific antibody may be more or less active in highly oxygenated blood, such as, for example, after passage through the lung or in the lower pH environments found in the tumor microenvironment. The conditionally active multi-specific antibody may be used as a drug, a therapeutic agent or a diagnostic agent.

Without wishing to be limited by theory, the conditionally active multi-specific antibody of the present disclosure binds to both the target cell and tumor-reactive lymphocyte to thereby bring the target cell in close proximity to the tumor-reactive lymphocyte. This is believed to facilitate an attack by the tumor-reactive lymphocyte on the target cell to thereby inhibit, damage or destroy the target cell. A therapeutic effect of inhibition or removing tumor cells may be achieved by using the conditionally active multi-specific antibody of the present disclosure to bring the reactive lymphocyte to tumor cells for inhibition, destruction and removal of the tumor cells from the subject.

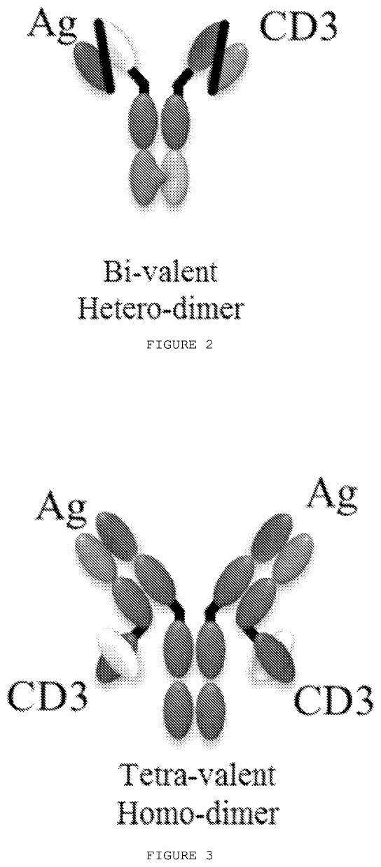

The structure/format of the multi-specific antibody may be any one of the structures/formats described in Brinkmann and Kontermann, “The making of bispecific antibodies,” MABs, vol. 9, pp. 182-212, 2017. Specifically, FIG. 2 of Brinkmann and Kontermann describes 19 different structures/formats for bispecific antibodies. These structures/formats include: (1) bispecific antibody conjugates; (2) hybrid bispecific IgG2; (3) “variable domain only” bispecific antibody molecules; (4) CH1/CL fusion proteins; (5) Fab fusion proteins; (6) non-immunoglobulin fusion proteins; (7) Fc-modified IgGs; (8) appended and Fc-modified IgGs; (9) modified Fe and CH3 fusion proteins; (10) appended IgGs-HC fusions; (11) appended IgGs-LC fusions; (12) appended IgGs-HC&LC fusions; (13) Fc fusions; (14) CH3 fusions; (15) IgE/IgM CH2 fusions; (16) F(ab′)₂ fusion; (17) CH1/CL fusion proteins; (18) modified IgGs; and (19) non-immunoglobulin fusions.

In particular embodiments, the multi-specific antibody may be a bi-valent scFv-Fc hetero-dimer as shown in FIG. 2 or a tetra-valent homodimer “butterfly” as shown in FIG. 3. In these two structures, the reactive lymphocyte antigens are not limited to CD3, which is only depicted as a representative of a tumor-reactive lymphocyte antigen. The multi-specific antibody of FIG. 2 has a first binding site to EpCAM, which is linked to a first heavy chain constant region (e.g., IgG) and a second binding site to a reactive lymphocyte antigen (e.g., CD3), which is linked to a second heavy chain constant region (e.g., IgG). The two heavy chains are engineered such that they can only form hetero dimers, for example, by using the knob-in-hole technique. The first and second binding sites are scFv antibodies binding to EpCAM and reactive lymphocyte antigen, respectively. Either one or both of the first and second binding sites have a conditionally active binding activity to the respective antigen.

The conditionally active multi-specific antibody of FIG. 3 may have a full-length IgG antibody binding to EpCAM and an scFv antibody binding to a reactive lymphocyte antigen (e.g., CD3). The scFv antibody is linked to the C terminus of the light chain of the IgG antibody via a linker. The linker may be a short Alanine linker (Ala)ₙ, a Serine linker (Ser)ₙ, a hydrophilic linker or a glycine-serine-rich linker. The heavy chain of the IgG antibody pairs with the light chain of the IgG antibody that has been linked to the scFv antibody, thus forming half of the homo-dimer. This multi-specific antibody has a “butterfly” configuration.

In some embodiments, the multi-specific antibody comprises an IgG antibody or fragment thereof that binds to a tumor-reactive lymphocyte antigen and a single chain antibody that binds to EpCAM, also forming a “butterfly” configuration as shown in FIG. 3. The single chain antibody may be an scFv antibody. The scFv antibody may be attached to a C terminus of the IgG antibody via a linker as described herein.

The binding sites of the multi-specific antibody of the disclosure each comprise a light chain variable region and a heavy chain variable region. The light chain variable region and the heavy chain variable region may be a single chain antibody format or may be a two-chain format as formed by pairing of a light chain and heavy chain (FIGS. 2 & 3). In a binding site that has conditional activity, one of the light chain and heavy chain variable regions is conditionally active or both may be conditionally active. An exemplary conditionally active anti-CD3 scFv antibody comprises a light chain variable region of SEQ ID NO: 101 and a heavy chain variable region of SEQ ID NO: 100. Additional sequences of conditionally active and non-conditionally active anti-CD3 antibodies useful in the multi-specific antibodies of the present disclosure can be found in WO 2019/241216 the disclosure of which is hereby incorporated by reference herein.

Exemplary conditionally active anti-EpCAM antibody variable regions that can be used to construct the multi-specific antibodies of the present disclosure include the heavy chain variable region of SEQ ID NO: 52 in combination with any one of the light chain variable regions of SEQ ID NOs: 53-69, and the light chain variable region of SEQ ID NO: 51 in combination with any one of the heavy chain variable regions of SEQ ID NOs: 70-96.

In some other embodiments, the multi-specific antibody may be constructed as shown in FIG. 2, having two variable regions forming the binding site for EpCAM and two other variable regions forming the binding site for the reactive lymphocyte antigen (e.g. CD3). These variable regions may be selected from the light chain and heavy chain variable regions having the amino acid sequences provided herein. One or both of the binding sites must have a conditional activity to their respective antigen. At each binding site having conditional activity, at least one of the light chain variable region and the heavy chain variable region has an increased affinity to its antigen at the first physiological condition (e.g., aberrant condition) as compared to the affinity at the second physiological condition (e.g., normal physiological condition). Thus, a person skilled in the art may select proper light chain variable regions and heavy chain variable regions to construct the multi-specific antibodies as shown in FIG. 2. The heavy chain fragments in FIG. 2 are selected from constant regions of IgG antibodies, including any subclass of IgG: IgG1, IgG2, IgG3 and IgG4.

In some other embodiments, the multi-specific antibody may be constructed as shown in FIG. 3. Similarly, the light chain variable region and heavy chain variable region in the scFv antibody and the light chain variable region and heavy chain variable region in the full-length IgG antibody may also be selected from the light chain and heavy chain variable regions having the amino acid sequences provided herein. At each binding site having conditional activity, at least one of the light chain variable region and the heavy chain variable region has an increased affinity to its antigen at the first physiological condition (e.g., aberrant condition) as compared to the affinity at the second physiological condition (e.g., normal physiological condition). Thus, a person skilled in the art may select proper light chain variable regions and heavy chain variable regions provided herein to construct the multi-specific antibodies shown in FIG. 3. The constant regions in FIG. 3 are selected from constant regions of IgG antibodies, including any subclass of IgG: IgG1, IgG2, IgG3 and IgG4.

Conditionally active anti-EpCAM antibodies or antibody fragments of the present disclosure are expected to exhibit reduced side-effects, relative to non-conditionally active anti-EpCAM antibodies, due to their reduced binding affinity to EpCAM in the normal tissue microenvironment. Anti-EpCAM antibodies or antibody fragments of the present disclosure are also expected to have a comparable efficacy to monoclonal anti-EpCAM antibodies known in the art. This combination of features permits use of a higher dosage of these anti-EpCAM antibodies or antibody fragments due to the reduced side effects, which may provide a more effective therapy option.

In some embodiments, the aberrant condition is an acidic pH in the range of from about 5.0 up to 7.0, or from about 5.2 to about 6.8, or from about 5.4 to about 6.8, or from about 5.6 to about 6.8, or from about 5.8 to about 6.8, or from about 6.0 to about 6.8, or from about 6.2 to about 6.8, or from about 6.4 to about 6.8, or from about 6.6 to about 6.8. In some embodiments, the acidic pH may be in the range of from about 6.4 up to 7.0, or from about 6.6 up to 7.0, or from about 6.8 up to 7.0. The normal physiological condition may be a normal physiological pH in the blood, which is well-established in the art. In some embodiments, the normal physiological pH in the blood may be in the range of from 7.0 to about 7.8, or from about 7.1 to about 7.7, or from about 7.2 to about 7.6, or from about 7.2 to about 7.5, or from about 7.2 to about 7.4.

In certain embodiments, the multi-specific antibody of the present disclosure has a ratio of the affinity or avidity to EpCAM and/or tumor-reactive lymphocyte antigen (e.g. CD3) at the aberrant condition to the same affinity or avidity at the normal physiological condition of at least about 1.3:1, or at least about 2:1, or at least about 3:1, or at least about 4:1, or at least about 5:1, or at least about 6:1, or at least about 7:1, or at least about 8:1, or at least about 9:1, or at least about 10:1, or at least about 11:1, or at least about 12:1, or at least about 13:1, or at least about 14:1, or at least about 15:1, or at least about 16:1, or at least about 17:1, or at least about 18:1, or at least about 19:1, or at least about 20:1, or at least about 30:1, or at least about 40:1, or at least about 50:1, or at least about 60:1, or at least about 70:1, or at least about 80:1, or at least about 90:1, or at least about 100:1.

In some embodiments, the antibody comprises one or more non-naturally occurring amino acids. For example, the non-naturally occurring amino acid comprises a carbonyl group, an acetyl group, an aminooxy group, a hydrazine group, a hydrazide group, a semicarbazide group, an azide group, or an alkyne group. See, e.g., U.S. Pat. No. 7,632,924 for suitable non-naturally occurring amino acids. The term “non-naturally occurring amino acid” also includes amino acids produced by modification (e.g. post-translational modifications) of a naturally occurring amino acid but are not themselves naturally incorporated into a growing polypeptide chain by the translation complex of a living organism. Examples of such non-naturally occurring amino acids include, but are not limited to, N-acetylglucosaminyl-L-serine, N-acetylglucosaminyl-L-threonine, and O-phosphotyrosine.

In some embodiments, the antibody is in a “mimetic” or “peptidomimetic” form, which is either entirely composed of synthetic, non-natural analogues of amino acids, or is a chimeric molecule of partly natural occurring amino acids and partly non-natural analogs of amino acids. The mimetic can also incorporate any amount of natural occurring amino acid conservative substitutions as long as such substitutions also do not substantially alter the antibody's structure and/or activity.

The mimetic form can contain any combination of non-natural structural components. In one aspect, mimetic of the disclosure includes one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, a gamma turn, a beta sheet, an alpha helix conformation, and the like. For example, the multi-specific antibody can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., ~C(═O)~CH2~ for —C(═O)~NH—), aminomethylene (CH₂—NH), ethylene, olefin (CH═CH), ether (CH2~O), thioether (CH₂~S), tetrazole, thiazole, retroamide, thioamide and ester (see, e.g., Spatola (1983) in Chemistry and Biochemistry of Amino Acids, Peptides and Proteins, vol. 7, pp 267-357, “Peptide Backbone Modifications,” in Chemistry and Biochemistry of Amino Acids, Peptides, and Proteins, vol. 7, B. Weinstein, ed., New York: Marcell Dekker, pp. 257-267).

More examples of non-naturally occurring amino acid residues include D- or L-naphthylalanine; D- or L-phenylglycine; D- or L-2 thienylalanine; D- or L-1, -2, -3, or -4 pyrenylalanine; D- or L-3 thienylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; D- or L-2-indole(alkyl)alanines; and, D- or L-alkylanines, where alkyl can be substituted or unsubstituted methyl, ethyl, propyl, hexyl, butyl, pentyl, isopropyl, iso-butyl, sec-isobutyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Acidic non-natural amino acids may be generated by substitution by, e.g., non-carboxylate amino acids while maintaining a negative charge, such as (phosphono)alanine; sulfated threonine. Carboxyl side groups (e.g., aspartyl or glutamyl) can also be selectively modified by reaction with carbodiimides (R′~N—C—N—R′) such as, e.g., 1-cyclohexyl-3(2-morpholinyl-(4-ethyl) carbodiimide or 1-ethyl-3(4-azonia-4,4-dimethylpentyl) carbodiimide. Aspartyl or glutamyl can also be converted to asparaginyl and glutaminyl residues by reaction with ammonium ions.

Basic non-natural amino acids can be generated by substitution with, e.g., (in addition to lysine and arginine) ornithine, citrulline, or (guanidino)-acetic acid, or (guanidino)alkyl-acetic acid, where alkyl is as defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

The mimetic form of the antibody may also contain one or more amino acids of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, and these forms can also be referred to as the R- or S-forms.

The mimetic form of the antibody may be synthesized using any protein chemical synthesis techniques. In a typical in vitro protein synthesis process, a peptide is extended in length by one amino acid through forming a peptide bond between the peptide and an amino acid. The formation of the peptide bond is carried out using a ligation reaction, which can use a natural amino acid or a non-natural amino acid. Thus, in this manner non-natural amino acids can be introduced into the antibody of the present disclosure to make mimetics.

In some embodiments, the non-naturally occurring amino acid in the antibody can provide for linkage to a macromolecule such as a polymer, a protein, or a fatty acid, etc. In some embodiments, the multi-specific antibody is linked (e.g., covalently linked) to a polymer (e.g., a polymer other than a polypeptide). Suitable polymers include, e.g., biocompatible polymers, water-soluble biocompatible polymers, synthetic polymers and naturally-occurring polymers. Examples of polymers include substituted or unsubstituted straight or branched chain polyalkylene, polyalkenylene and polyoxyalkylene polymers as well as branched or unbranched polysaccharides, e.g. a homo- or hetero-polysaccharide. More examples of suitable polymers include ethylene vinyl alcohol copolymers (commonly known by the generic name EVOH or by the trade name EVAL); polybutylmethacrylates; poly(hydroxyvalerates); poly(L-lactic acids); polycaprolactones; poly(lactide-co-glycolides); poly(hydroxybutyrates); poly(hydroxybutyrate-co-valerates); polydioxanones; polyorthoesters; polyanhydrides; poly(glycolic acids); poly(D,L-lactic acids); poly(glycolic acid-co-trimethylene carbonates); polyphosphoesters; polyphosphoester urethanes; poly(amino acids); cyanoacrylates; poly(trimethylene carbonates); poly(iminocarbonates); copoly(ether-esters) (e.g., poly(ethylene oxide)-poly(lactic acid) (PEO/PLA) co-polymers); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylenes and ethylene-alpha olefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitriles; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon-66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; amorphous Teflon™; poly(ethylene glycol); and carboxymethyl cellulose.

Examples of synthetic polymers include unsubstituted and substituted straight or branched chain poly(ethyleneglycol)s, poly(propyleneglycols) poly(vinylalcohols), and derivatives thereof, e.g., substituted poly(ethyleneglycols) such as methoxypoly(ethyleneglycol), and derivatives thereof. Suitable naturally-occurring polymers include, e.g., albumin, amylose, dextran, glycogen, and derivatives thereof.

The linked polymers can have an average molecular weight in a range of from 500 Da to 50000 Da, e.g., from 5000 Da to 40000 Da, or from 25000 to 40000 Da. For example, in some embodiments, where the multi-specific antibody comprises a poly(ethylene glycol) (PEG) or methoxypoly(ethyleneglycol) polymer, the PEG or methoxypoly(ethyleneglycol) polymer can have a molecular weight in a range of from about 0.5 kiloDaltons (kDa) to 1 kDa, from about 1 kDa to 5 kDa, from 5 kDa to 10 kDa, from 10 kDa to 25 kDa, from 25 kDa to 40 kDa, or from 40 kDa to 60 kDa.

For example, a water-soluble polymer (e.g., PEG) can be linked to the antibody by reacting a water-soluble polymer comprising a carbonyl group with an antibody having a non-naturally occurring amino acid that comprises an aminooxy, hydrazine, hydrazide or semicarbazide group. As another example, the antibody can be linked to a water-soluble polymer by reacting an antibody that comprises an alkyne-containing amino acid with a water-soluble polymer that comprises an azide moiety. In some cases, the azide or alkyne group is linked to the PEG molecule through an amide linkage.

In some embodiments, the macromolecule linked to the antibody is an albumin. The albumin may be for example the albumin of the subject that receives the antibody. For example, if antibody is intended to be used in a human, a human albumin may be linked to the antibody. If the antibody is intended to be used in a dog, a dog albumin may be linked to the multi-specific antibody. Generally speaking, an albumin from a species is linked to the antibody if the antibody is intended to be used in that species.

Examples of the linkers for conjugating the macromolecule to the antibody include glutaraldehyde, a homobifunctional cross-linker, or a heterobifunctional cross-linker Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a one-step reaction procedure in which the cross-linker is added to a solution containing a mixture of the macromolecule and antibody to be linked. In a mild alkaline pH, imido esters react only with primary amines to form imidoamides, and the overall charge of the cross-linked macromolecule and antibody is not affected. Homobifunctional sulfhydryl reactive cross-linkers include bismaleimidohexane (BMH), 1,5-difluoro-2,4-dinitrobenzene (DFDNB), and 1,4-di-(3′,2′-pyridyldithio) propinoamido butane (DPDPB).

Heterobifunctional cross-linkers have two or more different reactive moieties (e.g., an amine reactive moiety and a sulfhydryl-reactive moiety) and are cross-linked with one of the macromolecule and antibody via the amine or sulfhydryl reactive moiety, then reacted with the other one of macromolecule and antibody via the non-reacted moiety. Multiple heterobifunctional haloacetyl cross-linkers are available, as are pyridyl disulfide cross-linkers. Carbodiimides are a classic example of heterobifunctional cross-linking reagents for coupling carboxyl groups to amines, which results in an amide bond.

The antibody can be glycosylated, e.g., covalently linked to a carbohydrate or polysaccharide moiety. Glycosylation of multi-specific antibody is typically through N-linking or O-linking.

N-linking glycosylation refers to the attachment of the carbohydrate moiety to the side chain of an asparagine residue of the antibody. The tripeptide sequences “asparagine-X-serine” or “asparagine-X-threonine,” where X is any amino acid except proline, are the recognition sequences for enzymatic attachment of the carbohydrate moiety to the asparagine side chain. Thus, the presence of either of these tripeptide sequences in the antibody creates a potential glycosylation site. 0-linking glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody may be accomplished by altering its amino acid sequence such that it contains one or more of the above-described tripeptide sequences (for N-linking glycosylation sites). The alteration may also be made by the addition of, or substitution by, one or more serine or threonine residues to the sequence of the original antibody (for O-linking glycosylation sites). Conversely, removal of glycosylation sites can be accomplished by amino acid alteration within the native glycosylation sites of the multi-specific antibody.

The antibody can be covalently linked to another macromolecule (e.g., a lipid, a polypeptide, a synthetic polymer, a carbohydrate, and the like) using a linker selected from glutaraldehyde, a homobifunctional cross-linker, or a heterobifunctional cross-linker. Glutaraldehyde cross-links multi-specific antibody via their amino moieties. The homobifunctional cross-linkers and heterobifunctional cross-linkers are described in this application.

The linker used to construct the multi-specific antibodies may be a flexible peptide that ensures proper folding of the multi-specific antibodies. Exemplary linkers include (Ser)n, (Ser-Ala)n and (Ala)n.

In certain embodiments, the conditionally active anti-EpCAM antibodies of the present disclosure in addition to binding to human EpCAM also bind to EpCAM of non-human primates, for example, cynomolgus monkeys (Macaca fascicularis). The ability to bind to both human and non-human primate EpCAM proteins is advantageous in safety and efficacy testing as it allows for early testing in non-human primates rather than human subjects. In one particular embodiment, the anti-EpCAM antibodies, including bispecific antibodies and bind to each of human and cynomolgus EpCAM proteins with at least 5 times, at least 4 times, or at least 3 times greater affinity than the same antibodies bind rat or mouse EpCAM proteins. In another embodiment, a conditionally active bispecific antibody of the present disclosure binds each of human EpCAM and cynomolgus EpCAM proteins with at least 5 times greater affinity than the same antibody binds mouse or rat EpCAM proteins. In still another embodiment, these antibodies bind cynomolgus EpCAM protein with at least 45% or at least 50% of the binding affinity of the same antibody to human EpCAM protein.

In each of the embodiments disclosed herein, the antibody or antibody fragment may have a higher antigen binding activity to an EpCAM protein at a value of a condition in a tumor microenvironment in comparison with a different value of the same condition that occurs in a non-tumor microenvironment. In one embodiment, the condition is pH.

The conditionally active anti-EpCAM antibody or antibody fragment may have at least 70% of the antigen binding activity at pH 6.0 as compared to the same antigen binding activity of a parent antibody or antibody fragment from which it is derived, also at pH 6.0 and the antibody or antibody fragment may have less than 50%, or less than 40%, or less than 30%, or less than 20% or less than 10% of the antigen binding activity at pH 7.4 as compared to the same antigen binding activity of a parent antibody or antibody fragment from which it is derived at pH 7.4. The antigen binding activity may be, for example, binding to EpCAM protein or binding to CD3.

In each of the previous embodiments, the antigen binding activity may be measured by an ELISA assay.

In deriving these variants, one is guided by the process as described herein. The variants of the heavy chain and light chain variable regions may be prepared by introducing appropriate modifications into the nucleotide sequence encoding the heavy and light chain variable regions, or by peptide synthesis. Such modifications include, for example, deletions from, and/or insertions into and/or substitutions of residues within the amino acid sequences of the heavy and light chain variable regions. Any combination of deletion, insertion, and substitution can be made to arrive at the antibodies or antibody fragments of the present disclosure, provided that they possess the desired characteristics, e.g., antigen-binding to human EpCAM and/or conditional activity.

In certain embodiments, antibody or antibody fragment variants having one or more amino acid substitutions are provided. Sites of interest for substitutional mutagenesis include the CDRs and framework regions (FRs). Conservative substitutions are shown in Table 2 under the heading of “preferred substitutions.” More substantial changes are provided in Table 2 under the heading of “exemplary substitutions,” and as further described below in reference to amino acid side chain classes. Amino acid substitutions may be introduced into an antibody or antibody fragment of interest and the products screened for a desired activity, e.g., retained/improved antigen binding, or decreased immunogenicity.

TABLE 2
Amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more complementarity determining region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol., vol. 207, pp. 179-196, 2008), and/or SDRs (a-CDRs), with the resulting variant V_{H} or V_{L} being tested for binding affinity. Affinity maturation by constructing and reselecting from secondary libraries has been described, e.g., in Hoogenboom et al. in Methods in Molecular Biology, vol. 178, pp. 1-37, 2001). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody or antibody fragment to bind antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant V_{H} and V_{L} sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, vol. 244, pp. 1081-1085, 1989. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody or antibody fragment with antigen is affected. Further substitutions may be introduced at the amino acid locations demonstrating functional sensitivity to the initial substitutions. Alternatively, or additionally, a crystal structure of an antigen-antibody complex to identify contact points between the antibody or antibody fragment and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody include the fusion to the N- or C-terminus of the antibody to an enzyme (e.g. for ADEPT) or a polypeptide which increases the serum half-life of the antibody.

Amino acid sequence modification(s) of the antibodies described herein are contemplated. For example, it may be desirable to improve the binding affinity and/or other biological properties of the antibody. It is known that when a humanized antibody is produced by simply grafting only CDRs in V_{H} and V_{L} of an antibody derived from a non-human animal in FRs of the V_{H} and V_{L} of a human antibody, the antigen binding activity is reduced in comparison with that of the original antibody derived from a non-human animal. It is considered that several amino acid residues of the V_{H} and V_{L} of the non-human antibody, not only in CDRs but also in FRs, are directly or indirectly associated with the antigen binding activity. Hence, substitution of these amino acid residues with different amino acid residues derived from FRs of the V_{H} and V_{L} of the human antibody would reduce of the binding activity. In order to resolve the problem, in antibodies grafted with human CDR, attempts have to be made to identify, among amino acid sequences of the FR of the V_{H} and V_{L} of human antibodies, an amino acid residue which is directly associated with binding to the antibody, or which interacts with an amino acid residue of CDR, or which maintains the three-dimensional structure of the antibody and which is directly associated with binding to the antigen. The reduced antigen binding activity could be increased by replacing the identified amino acids with amino acid residues of the original antibody derived from a non-human animal.

Modifications and changes may be made in the structure of the antibodies of the present disclosure, and in the DNA sequences encoding them, and still obtain a functional molecule that encodes an antibody with desirable characteristics.

In making the changes in the amino sequences, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

A further object of the present disclosure also encompasses function-conservative variants of the antibodies of the present disclosure.

Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical) over the whole length of the shorter sequence. Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the sequences of the antibodies or antibody fragments of the disclosure, or corresponding DNA sequences which encode said antibodies or antibody fragments, without appreciable loss of their biological activity.

It is known in the art that certain amino acids may be substituted by other amino acids having a similar hydropathic index or score and still result in a protein with similar biological activity, i.e. still obtain a biological functionally equivalent protein.

As outlined herein, amino acid substitutions are generally, therefore, based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include arginine and lysine; glutamate and aspartate; serine and threonine; glutamine and asparagine; and valine, leucine and isoleucine.

In certain embodiments, the anti-EpCAM antibodies or antibody fragments provided herein may be altered to increase or decrease the extent to which the antibodies or antibody fragments are glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH, vol. 15, pp. 26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the disclosure may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol., vol. 336, pp. 1239-1249, 2004; Yamane-Ohnuki et al. Biotech. Bioeng., vol. 87, pp. 614-622, 2004. Examples of cell lines capable of producing defucosylated antibodies include Lec13 CHO cells deficient in protein fucosylation (Ripka et al. Arch. Biochem. Biophys., vol. 249, pp. 533-545, 1986; US Pat Appl No US 2003/0157108 A; and WO 2004/056312 A1, especially at Example 11), and knockout cell lines, such as alpha-1,6-fucosyltransferase gene, FUT8, knockout CHO cells (see, e.g., Yamane-Ohnuki et al. Biotech. Bioeng., vol. 87, pp. 614-622, 2004; Kanda, Y. et al., Biotechnol. Bioeng., vol. 94, pp. 680-688, 2006; and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In certain embodiments, one or more amino acid modifications may be introduced into the Fe region of the anti-EpCAM antibodies or antibody fragments provided herein, thereby generating an Fe region variant. The Fe region variant may comprise a human Fe region sequence (e.g., a human IgG1, IgG2, IgG3 or IgG4 Fe region) comprising an amino acid modification (e.g. a substitution) at one or more amino acid positions.

Certain embodiments contemplate an antibody variant that possesses some but not all effector functions, which make it a desirable candidate for applications in which the half-life of the antibody in vivo is important yet certain effector functions (such as ADCC) are unnecessary or deleterious. In vitro and/or in vivo cytotoxicity assays can be conducted to confirm the reduction/depletion of CDC and/or ADCC activities. For example, Fe receptor (FcR) binding assays can be conducted to ensure that the antibody lacks FcγR binding (hence likely lacking ADCC activity) but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol., vol. 9, pp. 457-492, 1991. Non-limiting examples of in vitro assays to assess ADCC activity of a molecule of interest is described in U.S. Pat. No. 5,500,362 (see also, e.g. Hellstrom et al. Proc. Nat'l Acad. Sci. USA, vol. 83, pp. 7059-7063, 1986) and Hellstrom, I et al., Proc. Nat'l Acad. Sci. USA, vol. 82, pp. 1499-1502, 1985; U.S. Pat. No. 5,821,337 (see also Bruggemann et al., J. Exp. Med., vol. 166, pp. 1351-1361, 1987). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA, vol. 95, pp. 652-656, 1998. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods, vol. 202, pp. 163-171, 1996; Cragg, M. S. et al., Blood, vol. 101, pp. 1045-1052, 2003; and Cragg, M. S, and M. J. Glennie, Blood, vol. 103, pp. 2738-2743, 2004). FcRn binding and in vivo clearance/half-life determinations can also be performed using methods known in the art (see, e.g., Petkova, S. B. et al., Int'l. Immunol., vol. 18, pp. 1759-1769, 2006).

Variants of the antibodies or antibody fragments with reduced effector function include those with substitution of one or more of Fe region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fe mutant with substitution of residues 265 and 297 to alanine (U.S. Pat. No. 7,332,581).

Certain antibody variants with improved or diminished binding to FcRs are described in the art. (See, e.g., U.S. Pat. No. 6,737,056; WO 2004/056312, and Shields et al., J. Biol. Chem., vol. 9, pp. 6591-6604, 2001).

In certain embodiments, an antibody variant comprises an Fe region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol., vol. 164, pp. 4178-4184, 2000.

Antibodies with increased half-lives and improved binding to the neonatal Fe receptor (FcRn), which is responsible for the transfer of maternal IgGs to the fetus (Guyer et al., J. Immunol., vol. 117, pp. 587-593, 1976 and Kim et al., J. Immunol., vol. 24, p. 249, 1994), are described in US2005/0014934. Those antibodies comprise an Fc region with one or more substitutions therein which improve binding of the Fc region to FcRn. Such Fe variants include/e those with substitutions at one or more of Fe region residues: 238, 256, 265, 272, 286, 303, 305, 307, 311, 312, 317, 340, 356, 360, 362, 376, 378, 380, 382, 413, 424 or 434, e.g., substitution of Fe region residue 434 (U.S. Pat. No. 7,371,826). See also Duncan & Winter, Nature, vol. 322, pp. 738-740, 1988; U.S. Pat. Nos. 5,648,260; 5,624,821; and WO 94/29351 concerning other examples of Fe region variants.

In certain embodiments, it may be desirable to create cysteine engineered antibodies, e.g., “thioMAbs,” in which one or more residues of the anti-EpCAM antibodies or antibody fragments are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fe region. Cysteine engineered antibodies may be generated as described, e.g., in U.S. Pat. No. 7,521,541.

In certain embodiments, the anti-EpCAM antibodies or antibody fragments provided herein may be further modified to contain additional non-proteinaceous moieties that are known in the art and readily available. The moieties suitable for derivatization of the antibody or antibody fragment include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody or antibody fragment may vary, and if more than one polymer are attached, they can be the same or different molecules. In general, the number and/or type of polymers used for derivatization can be determined based on considerations including, but not limited to, the particular properties or functions of the antibody or antibody fragment to be improved, whether the derivative will be used in a therapy under defined conditions, etc.

The anti-EpCAM antibodies or antibody fragments of the disclosure, or their variants, have a higher binding affinity to EpCAM under a condition in a tumor microenvironment than under a condition in a non-tumor microenvironment. In one embodiment, the condition in tumor microenvironment and the condition in a non-tumor microenvironment are both pH. In one embodiment, the anti-EpCAM antibodies or antibody fragments of the disclosure thus selectively bind to EpCAM at a pH about 5.0-6.8 but will have a lower binding affinity to EpCAM at a pH about 7.2-7.8 encountered in a normal non-tumor microenvironment. In another embodiment, the anti-EpCAM antibodies or antibody fragments of the disclosure selectively bind to EpCAM at a pH of about 5.0-6.9 but will have a lower binding affinity to EpCAM at a pH of about 7.0-7.6 encountered in a normal non-tumor microenvironment. In still another embodiment, the anti-EpCAM antibodies or antibody fragments of the disclosure selectively bind to EpCAM at a pH of about 5.0-6.9 but will have a lower binding affinity to EpCAM at a pH of about 7.0-7.8 encountered in a normal non-tumor microenvironment. In yet another embodiment, the anti-EpCAM antibodies or antibody fragments of the disclosure selectively bind to EpCAM at a pH of about 5.0-6.8 but will have a lower binding affinity to EpCAM at a pH of about 7.2-7.6 encountered in a normal non-tumor microenvironment. In one embodiment, the anti-EpCAM antibodies or antibody fragments have higher binding affinity to EpCAM at pH 6.0 than at pH 7.4 in screening assays such as those described herein.

In certain embodiments, the anti-EpCAM antibodies or antibody fragments of the present disclosure have a dissociation constant (Kd) with EpCAM under a condition in tumor microenvironment of about ≤1 μM, ≤100 nM, ≤10 nM, ≤1 nM, ≤0.1 nM, ≤0.01 nM, or ≤0.001 nM (e.g. 10⁻⁸ M or less, or from 10⁻⁸M to 10⁻¹³M, or from 10⁻⁹M to 10⁻¹³ M). In one embodiment, the ratio of the Kd of the antibody or antibody fragment with EpCAM at the condition in tumor microenvironment to the Kd at the same condition in non-tumor microenvironment is at least about 1.5:1, at least about 2:1, at least about 3:1, at least about 4:1, at least about 5:1, at least about 6:1, at least about 7:1, at least about 8:1, at least about 9:1, at least about 10:1, at least about 20:1, at least about 30:1, at least about 50:1, at least about 70:1, or at least about 100:1. In another embodiment, the ratio of the Kd of the antibody or antibody fragment with EpCAM at the condition in tumor microenvironment to the Kd at the same condition in non-tumor microenvironment is at least about 1.5:1, at least about 2:1, at least about 3:1, at least about 4:1, at least about 5:1, at least about 6:1, at least about 7:1, at least about 8:1, at least about 9:1, at least about 10:1, at least about 15:1 or at least about 20:1.

In one embodiment, Kd is measured by a radiolabeled antigen binding assay (RIA) performed with the Fab version of an antibody of interest and its antigen using the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol.293:865-881 (1999)). To establish conditions for the assay, MICROTITER® multi-well plates (Thermo Scientific) are coated overnight with 5 g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 l/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at about 10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (~0.2 μM) before injection at a flow rate of 5 l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

The anti-EpCAM antibodies of the disclosure may be a chimeric, humanized or human antibody. In one embodiment, an anti-EpCAM antibody fragment is employed, e.g., a Fv, Fab, Fab′, Fab′-SH, scFv, a diabody, a triabody, a tetrabody or an F(ab′)₂ fragment and multispecific antibodies formed from antibody fragments. In another embodiment, the antibody is a full length antibody, e.g., an intact IgG antibody or other antibody class or isotype as defined herein. For a review of certain antibody fragments, see Hudson et al. Nat. Med., vol. 9, pp. 129-134, 2003. For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

The diabodies of the disclosure may be bivalent or bispecific. See, for example, EP 404,097; WO 1993/01161; Hudson et al., Nat. Med. 9:129-134 (2003); and Hollinger et al., Proc. Natl. Acad. Sci. USA, vol. 90, pp. 6444-6448, 1993 for examples of diabodies. Examples of triabodies and tetrabodies are also described in Hudson et al., Nat. Med., vol. 9, pp. 129-134, 2003.

In some embodiments, the disclosure provides single-domain antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage).

In some embodiments, the anti-EpCAM antibodies of the disclosure may be chimeric antibodies. Certain chimeric antibodies are described, e.g., in U.S. Pat. No. 4,816,567; and Morrison et al., Proc. Natl. Acad. Sci. USA, vol. 81, pp. 6851-6855, 1984). In one example, the chimeric antibody comprises a non-human variable region (e.g., a variable region derived from a mouse, rat, hamster, rabbit, or non-human primate, such as a monkey) and a human constant region. In a further example, the chimeric antibody is a “class switched” antibody in which the class or subclass of the antibody has been changed relative to the class or subclass of the parent antibody. Chimeric antibodies include antigen-binding fragments thereof.

In certain embodiments, the chimeric antibody of the disclosure is a humanized antibody. Typically, such a non-human antibody is humanized to reduce immunogenicity to humans, while retaining the specificity and affinity of the parental non-human antibody. Generally, a humanized antibody comprises one or more variable domains in which CDRs (or portions thereof) are derived from a non-human antibody, and FRs (or portions thereof) are derived from human antibody sequences. A humanized antibody may optionally also comprise at least a portion of a human constant region. In some embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci., vol. 13, pp. 1619-1633, 2008, and are further described, e.g., in Riechmann et al., Nature, vol. 332, pp. 323-329, 1988; Queen et al., Proc. Nat'l Acad. Sci. USA, vol. 86, pp. 10029-10033, 1989; U.S. Pat. Nos. 5,821,337, 7,527,791, 6,982,321, and 7,087,409; Kashmiri et al., Methods, vol. 36, pp. 25-34, 2005 (describing SDR (a-CDR) grafting); Padlan, Mol. Immunol., vol. 28, pp. 489-498, 1991 (describing “resurfacing”); Dall'Acqua et al., Methods, vol. 36, pp. 43-60, 2005 (describing “FR shuffling”); and Osbourn et al., Methods, vol. 36, pp. 61-68, 2005 and Klimka et al., Br. J. Cancer, vol. 83, pp. 252-260, 2000 (describing the “guided selection” approach to FR shuffling).

Human framework regions that may be used for humanization include but are not limited to: framework regions selected using the “best-fit” method (see, e.g., Sims et al. J. Immunol., vol. 151, p. 2296, 1993); framework regions derived from the consensus sequence of human antibodies of a particular subgroup of light or heavy chain variable regions (see, e.g., Carter et al. Proc. Natl. Acad. Sci. USA, vol. 89, p. 4285, 1992; and Presta et al. J. Immunol., vol. 151, p. 2623, 1993); human mature (somatically mutated) framework regions or human germline framework regions (see, e.g., Almagro and Fransson, Front. Biosci., vol. 13, pp. 1619-1633, 2008); and framework regions derived from screening FR libraries (see, e.g., Baca et al., J. Biol. Chem., vol. 272, pp. 10678-10684, 1997 and Rosok et al., J. Biol. Chem., vol. 271, pp. 22611-22618, 1996).

In some embodiments, the anti-EpCAM antibodies of the disclosure are multispecific, e.g. bispecific antibodies. Multispecific antibodies are monoclonal antibodies that have binding specificities for at least two different sites. In certain embodiments, one of the binding specificities is for EpCAM and the other is for another antigen such as CD3. One specific example of this is a bispecific antibody with binding specificities for EpCAM and CD3+. A bispecific conditionally active antibody may be mono conditionally active or dual conditionally active. Thus, in the case of the mono conditionally active bispecific antibody, one of the binding sites is conditionally active and the other is not, e.g. a wild type (WT) EpCAM paired with a conditionally active (CAB) CD3+ or a CAB EpCAM paired with a WT CD3+. In the case of a dual conditionally active antibody, both binding sites are conditionally active as in CAB EpCAM×CAB CD3+.

Multispecific antibodies can be mono, dual, tri-, etc. conditionally active. Thus, any one or more of the binding regions of the multispecific antibody may be conditionally active.

In certain embodiments, bispecific antibodies may bind to two different epitopes of EpCAM. Bispecific antibodies may also be used to localize cytotoxic agents to cells which express EpCAM. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature, vol. 305, pp. 537-540, 1983), WO 93/08829, and Traunecker et al., EMBO J. vol. 10, pp. 3655-3659, 1991), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, vol. 229, pp. 81-83, 1985); using leucine zippers to produce bi-specific antibodies (see, e.g., Kostelny et al., J. Immunol., vol. 148, pp. 1547-1553, 1992); using “diabody” technology for making bispecific antibody fragments (see, e.g., Hollinger et al., Proc. Natl. Acad. Sci. USA, vol. 90, pp. 6444-6448, 1993); and using single-chain Fv (scFv) dimers (see, e.g. Gruber et al., J. Immunol., vol. 152, pp. 5368-5374, 1994); and preparing trispecific antibodies as described, e.g., in Tutt et al. J. Immunol., vol. 147, pp. 60-69, 1991.

Engineered antibodies with three or more functional antigen binding sites, including “Octopus antibodies,” are also included herein (see, e.g. US 2006/0025576A1).

The anti-EpCAM antibodies or antibody fragments of the disclosure may be produced using recombinant methods and compositions, which are described in detail in US 2016/0017040.

Bispecific antibodies of the present disclosure comprise an anti-EpCAM antibody disclosed herein in combination with an antibody that binds to a T-lymphocyte antigen. In one embodiment, the antibodies may comprise an anti-EpCAM portion which contains the combinations of complementarity determining regions provided in Table 1. In other embodiments, the anti-EpCAM portion of the bispecific antibody contains a heavy chain variable region of SEQ ID NO: 52 and a light chain variable region of any one of SEQ ID NOs: 53-69. In another embodiment, anti-EpCAM portion of the bispecific antibody contains a light chain variable region of SEQ ID NO: 51 and a heavy chain variable region of any one of SEQ ID NOs: 70-96. In some embodiments the antibody that binds to a T-lymphocyte antigen is a scFv antibody. In certain embodiments, the T-lymphocyte antigen antibody may be an anti-CD3 antibody. In particular embodiments, the anti-CD3 antibody contains a light chain variable region of SEQ ID NO: 101 and a heavy chain variable region of SEQ ID NO: 100. In a more particular embodiment, the anti-CD3 scFv antibody comprises SEQ ID NO: 99. Additional sequences of anti-CD3 antibodies useful in the multi-specific antibodies of the present disclosure can be found in WO 2019/241216.

In one embodiment, the bispecific antibody comprises an intact IgG molecule or fragment of an IgG antibody such as an (Fab′) 2 fragment with an scFv antibody attached to the C-terminus of the light chain of the IgG or IgG fragment as shown in FIG. 3. In one embodiment, the IgG portion of the bispecific antibody may comprise any of the anti-EpCAM antibodies disclosed herein and the scFv portion may be any of the anti-CD3 antibodies disclosed herein. In another embodiment, the IgG portion of the bispecific antibody may be any of the anti-CD3 antibodies disclosed herein and the scFV antibody may be any of the anti-EpCAM antibodies disclosed herein. In one particular embodiment, the IgG portion of the bispecific antibody comprises a light chain variable region containing SEQ ID NOs: 1-3 and a heavy chain variable region containing SEQ ID NO: 4, SEQ ID NO: 5 and SEQ ID NO: 45, while the scFv portion comprises a light chain variable region of SEQ ID NO: 101 and a heavy chain variable region of SEQ ID NO: 100. In another embodiment, the IgG portion of the bispecific antibody comprises a light chain variable region of SEQ ID NO: 51 and a heavy chain variable region of SEQ ID NO: 91, and a scFv fragment of SEQ ID NO: 99.

In certain embodiments, any of the anti-EpCAM antibodies or antibody fragments provided herein may be used for detecting the presence of EpCAM in a biological sample, either quantitatively or qualitatively. In certain embodiments, a biological sample comprises a cell or tissue, such as breast, pancreas, esophagus, lung and/or brain cells or tissue.

A further aspect of the disclosure relates to an anti-EpCAM antibody or antibody fragment of the disclosure for diagnosing and/or monitoring a cancer or another disease in which EpCAM expression levels are increased or decreased from a normal physiological level at least one location in the body.

In one embodiment, antibodies or antibody fragments of the disclosure may be labelled with a detectable molecule or substance, such as a fluorescent molecule, a radioactive molecule or any other label known in the art. For example, an antibody or antibody fragment of the disclosure may be labelled with a radioactive molecule. For example, suitable radioactive molecules include but are not limited to radioactive atoms used for scintigraphic studies such as ¹²³I, ¹²⁴I, ¹¹¹In, ¹⁸⁶Re, and ¹⁸⁸Re. Antibodies or antibody fragments of the disclosure may also be labelled with a spin label for nuclear magnetic resonance (NMR) imaging, such as iodine-123, iodine-131, indium-Ill, fluorine-19, carbon-13, nitrogen-15, oxygen-17, gadolinium, manganese or iron. Following administration of the antibody, the distribution of the radiolabeled antibody within the patient is detected. Any suitable known method can be used. Some non-limiting examples include, computed tomography (CT), position emission tomography (PET), magnetic resonance imaging (MRI), fluorescence, chemiluminescence and sonography.

Antibodies or antibody fragments of the disclosure may be useful for diagnosing and staging of cancer and diseases associated with EpCAM overexpression. Cancers associated with EpCAM overexpression may include squamous cell cancer, small-cell lung cancer, non-small cell lung cancer, gastric cancer, pancreatic cancer, glial cell tumors such as glioblastoma and neurofibromatosis, cervical cancer, ovarian cancer, liver cancer, bladder cancer, hepatoma, breast cancer, colon cancer, melanoma, colorectal cancer, endometrial carcinoma, salivary gland carcinoma, kidney cancer, renal cancer, prostate cancer, vulval cancer, thyroid cancer, hepatic carcinoma, sarcomas, hematological cancers (leukemias), astrocytomas, and various types of head and neck cancer or other EpCAM expressing or overexpressing hyperproliferative diseases.

Antibodies or antibody fragments of the disclosure may be useful for diagnosing diseases other than cancers for which EpCAM expression is increased or decreased. Both the soluble or cellular EpCAM forms can be used for such diagnoses. Typically, such diagnostic methods involve use of a biological sample obtained from the patient. The biological sample encompasses a variety of sample types obtained from a subject that can be used in a diagnostic or monitoring assay. Biological samples include but are not limited to blood and other liquid samples of biological origin, solid tissue samples such as a biopsy specimen or a tissue culture or cells derived therefrom, and the progeny thereof. For example, biological samples include cells obtained from a tissue sample collected from an individual suspected of having a cancer associated with EpCAM overexpression, and in preferred embodiments from glioma, gastric, lung, pancreatic, breast, prostate, renal, hepatic and endometrial. Biological samples encompass clinical samples, cells in culture, cell supernatants, cell lysates, serum, plasma, biological fluid, and tissue samples.

In a particular embodiment is provided a method of diagnosing a cancer associated with EpCAM overexpression in a subject by detecting EpCAM on cells from the subject using the antibody of the disclosure. In particular, said method may include steps of:
1. contacting a biological sample of a subject with an antibody or antibody fragment according to the disclosure under conditions suitable for the antibody or antibody fragment to form complexes with cells in the biological sample that express EpCAM; and
2. detecting and/or quantifying said complexes, whereby detection of said complexes is indicative of a cancer associated with EpCAM overexpression.

In order to monitor the progress of a cancer, the method may be repeated at different times, in order to determine if antibody binding to the samples increases or decreases, wherefrom it can be determined if the cancer has progressed, regressed or stabilized.

In a particular embodiment, the disclosure provides a method of diagnosing a disease associated with the expression or overexpression of EpCAM. Examples of such diseases may include cancers, human immune disorders, thrombotic diseases (thrombosis and atherothrombosis), and cardiovascular diseases.

In one embodiment, an anti-EpCAM antibody or antibody fragment for use in a method of diagnosis or detection is provided. In a further aspect, a method of detecting the presence of EpCAM4 in a biological sample is provided. In a further aspect, a method of quantifying the amount of EpCAM in a biological sample is provided. In certain embodiments, the method comprises contacting the biological sample with an anti-EpCAM antibody or antibody fragment as described herein under conditions permissive for binding of the anti-EpCAM antibody or antibody fragment to EpCAM and detecting whether a complex is formed between the anti-EpCAM antibody or antibody fragment and EpCAM. Such a method may be carried out in vitro or in vivo. In one embodiment, an anti-EpCAM4 antibody or antibody fragment is used to select subjects eligible for therapy. In some embodiments, the therapy will include administration of an anti-EpCAM antibody or antibody fragment to the subject.

In certain embodiments, labeled anti-EpCAM antibodies or antibody fragments are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

The anti-EpCAM antibodies or antibody fragments and multi-specific antibodies incorporating same have cell killing activity. This cell killing activity extends to multiple different types of cell lines. Thus, the anti-EpCAM antibodies, fragments or multi-specific antibodies thereof may be useful for treating proliferative diseases associated with EpCAM expression. The antibodies, fragments or multi-specific antibodies may be used alone or in combination with any suitable agent or other conventional treatments.

The anti-EpCAM antibody, antibody fragment or multi-specific antibody of the present disclosure may be used to treat diseases associated with EpCAM expression, overexpression or activation. There are no particular limitations on the types of cancer or tissue that can be treated other than the requirement for EpCAM expression. Examples include squamous cell cancer, small-cell lung cancer, non-small cell lung cancer, gastric cancer, pancreatic cancer, glial cell tumors such as glioblastoma and neurofibromatosis, cervical cancer, ovarian cancer, liver cancer, bladder cancer, hepatoma, breast cancer, colon cancer, melanoma, colorectal cancer, endometrial carcinoma, salivary gland carcinoma, kidney cancer, renal cancer, prostate cancer, vulval cancer, thyroid cancer, hepatic carcinoma, sarcomas, hematological cancers (leukemias), astrocytomas, and various types of head and neck cancer. Particular cancers are glioma, gastric, lung, pancreatic, breast, prostate, renal, hepatic and endometrial cancer.

Anti-EpCAM antibodies, antibody fragments or multi-specific antibodies of the present disclosure are potential activators of the innate immune response and thus may be used in the treatment of human immune disorders, such as sepsis. The anti-EpCAM antibody or antibody fragment of the disclosure may also be used as adjuvants for immunization such as for vaccines and as anti-infection agents against, for example, bacteria, viruses and parasites.

In each of the embodiments of the treatment methods described herein, the anti-EpCAM antibody, antibody fragment or multi-specific anti-EpCAM antibody or antibody fragment may be delivered in a manner consistent with conventional methodologies associated with management of the disease or disorder for which treatment is sought. In accordance with the disclosure herein, an effective amount of the antibody, antibody fragment or multi-specific antibody is administered to a subject in need of such treatment for a time and under conditions sufficient to prevent or treat the disease or disorder. Thus, an aspect of the disclosure relates to a method for treating a disease associated with the expression of EpCAM comprising administering to a subject in need thereof with a therapeutically effective amount of an antibody, antibody fragment or multi-specific antibody of the disclosure.

For administration, the anti-EpCAM antibody, antibody fragment or multi-specific antibody of the disclosure may be formulated as a pharmaceutical composition. The pharmaceutical composition including anti-EpCAM antibody, antibody fragment or multi-specific antibody can be formulated according to known methods for preparing pharmaceutical compositions. In such methods, the therapeutic molecule is typically combined with a mixture, solution or composition containing a pharmaceutically acceptable carrier.

A pharmaceutically acceptable carrier is a material that can be tolerated by a recipient patient. Sterile phosphate-buffered saline is one example of a pharmaceutically acceptable carrier. Other suitable pharmaceutically acceptable carriers are well-known to those in the art. (See, e.g., Gennaro (ed.), Remington's Pharmaceutical Sciences (Mack Publishing Company, 19th ed. 1995)) Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc. These considerations can be taken into account by a skilled person to formulate suitable pharmaceutical compositions. The pharmaceutical compositions of the disclosure can be formulated for topical, oral, parenteral, intranasal, intravenous, intramuscular, subcutaneous or intraocular administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition of, for example, sterilized water or physiological saline, permit the constitution of injectable solutions.

In some embodiments, tonicity agents, sometimes known as “stabilizers” are present to adjust or maintain the tonicity of a liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount of from 0.1% to 25% by weight, for example, 1 to 5% of the pharmaceutical composition. Tonicity agents may include polyhydric sugar alcohols, for example trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients may include: polyhydric sugar alcohols (enumerated herein); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be employed to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants may be present in a concentration range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The doses used for the administration can be adapted as a function of various parameters, and in particular as a function of the mode of administration used, of the relevant pathology, or alternatively of the desired duration of treatment. To prepare pharmaceutical compositions, an effective amount of the antibody, antibody fragment or multi-specific antibody may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi.

Solutions of the active compounds as free base or pharmacologically acceptable salts can be prepared in a water suitably mixed with a surfactant. Dispersions can also be prepared in glycerol, liquid polyethylene glycols, and mixtures thereof and in oils. Under ordinary conditions of storage and use, these preparations contain a preservative to prevent the growth of microorganisms.

The anti-EpCAM antibody, antibody fragment or multi-specific antibody of the present disclosure can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the anti-EpCAM antibodies, antibody fragments or multi-specific antibodies described herein in the required amount in the appropriate solvent with one or more of the other ingredients enumerated above, as may be required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of dimethyl sulfoxide (DMSO) as solvent is envisioned to result in extremely rapid penetration, delivering high concentrations of the active agents to a small tumor area.

Upon formulation, solutions containing the anti-EpCAM antibodies, antibody fragments or multi-specific antibodies may be administered in a manner compatible with the dosage formulation and in such amount as is therapeutically effective. The formulations are easily administered in a variety of dosage forms, such as the type of injectable solutions described herein.

For parenteral administration in an aqueous solution, for example, the solution should be suitably buffered if necessary, and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition, weight, and/or gender of the subject being treated. The person responsible for administration will, in any event, determine the appropriate dose for the individual subject.

The antibodies, antibody fragments or multi-specific antibodies disclosed herein may be formulated within a therapeutic mixture to deliver about 0.0001 to 10.0 milligrams, or about 0.001 to 5 milligrams, or about 0.001 to 1 milligrams, or about 0.001 to 0.1 milligrams, or about 0.1 to 1.0 or even about 10 milligrams per dose. Multiple doses can also be administered at selected time intervals.

In certain embodiments, the use of liposomes and/or nanoparticles is contemplated for the introduction of antibodies or antibody fragments into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 m) are generally designed using polymers able to degrade in vivo. Biodegradable polyalkyl-cyanoacrylate nanoparticles that meet these requirements are contemplated for use in the present disclosure, and such particles may be easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 A, containing an aqueous solution in the core. The physical characteristics of liposomes depend on pH, ionic strength and the presence of divalent cations.

Pharmaceutical formulations containing an anti-EpCAM antibody, antibody fragment or multi-specific antibody as described herein are prepared by mixing such antibody, antibody fragment or multi-specific antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated. Preferably, ingredients with complementary activities that do not adversely affect each other may be combined into a single formulation. For example, it may be desirable to provide an EGFR antagonist (such as erlotinib), an anti-angiogenic agent (such as a VEGF antagonist which may be an anti-VEGF antibody) or a chemotherapeutic agent (such as a taxoid or a platinum agent) in addition to the anti-EpCAM antibody, antibody fragment or multi-specific antibody of the present disclosure. Such active ingredients are suitably present in combination in amounts that are effective for the purpose intended.

Any of the anti-EpCAM antibodies, antibody fragments or multi-specific antibodies provided herein may be used in therapeutic methods. In one aspect, an anti-EpCAM antibody, antibody fragment or multi-specific antibody for use as a medicament is provided. In further aspects, an anti-EpCAM antibody or antibody fragment for use in treating cancer (e.g., breast cancer, non-small cell lung cancer, pancreatic cancer, brain cancer, cancer of pancreas, brain, kidney, ovary, stomach, leukemia, uterine endometrium, colon, prostate, thyroid, liver, osteosarcoma, and/or melanoma) is provided. In certain embodiments, an anti-EpCAM antibody, antibody fragment or multi-specific antibody for use in a method of treatment is provided. In certain embodiments, the disclosure provides an anti-EpCAM antibody, antibody fragment or multi-specific antibody for use in a method of treating an individual having cancer comprising administering to the individual an effective amount of the anti-EpCAM antibody, antibody fragment or multi-specific antibody. In further embodiments, this disclosure provides an anti-EpCAM antibody, antibody fragment or multi-specific antibody for use in inhibiting angiogenesis, inhibiting cell proliferation, inhibiting tumor vasculature (e.g., intratumoral vasculature or tumor-associated vasculature), and/or inhibiting tumor stromal function.

In a further aspect, the disclosure provides for the use of an anti-EpCAM antibody or antibody fragment in the manufacture or preparation of a medicament. In one embodiment, the medicament is for treatment of cancer (in some embodiments, breast cancer, non-small cell lung cancer, pancreatic cancer, brain cancer, cancer of the pancreas, brain, kidney, ovary, stomach, leukemia, uterine endometrium, colon, prostate, thyroid, liver, osteosarcoma, and/or melanoma). In a further embodiment, the medicament is for use in a method of treating cancer comprising administering to an individual having cancer an effective amount of the medicament. In one such embodiment, the method further comprises administering to the individual an effective amount of at least one additional therapeutic agent, e.g., as described herein. In a further embodiment, the medicament is for inhibiting angiogenesis, inhibiting cell proliferation, inhibiting immune function, inhibiting tumor vasculature (e.g., intratumoral vasculature or tumor-associated vasculature), and/or inhibiting tumor stromal function. In a further embodiment, the medicament is for use in a method of inhibiting angiogenesis, inhibiting cell proliferation, inhibiting tumor vasculature (e.g., intratumoral vasculature or tumor-associated vasculature), and/or inhibiting tumor stromal function in an individual comprising administering to the individual an amount effective of the medicament to inhibit angiogenesis, inhibit cell proliferation, promote immune function, induce inflammatory cytokine section (e.g., from tumor-associated macrophages), inhibit tumor vasculature development (e.g., intratumoral vasculature or tumor-associated vasculature), and/or inhibit tumor stromal function. An “individual” according to any of the above embodiments may be a human.

In a further aspect, the disclosure provides pharmaceutical formulations comprising any of the anti-EpCAM antibodies, antibody fragments or multi-specific antibodies provided herein, e.g., for use in any of the above therapeutic methods. In one embodiment, a pharmaceutical formulation comprises any of the anti-EpCAM antibodies, antibody fragments or multi-specific antibodies provided herein and a pharmaceutically acceptable carrier. In another embodiment, a pharmaceutical formulation comprises any of the anti-EpCAM antibodies, antibody fragments or multi-specific antibodies provided herein and at least one additional therapeutic agent, e.g., as described below.

In each and every treatment described herein, the antibodies, antibody fragments or multi-specific antibodies of the disclosure can be used alone or in combination with other agents in a therapy. For instance, an antibody of the disclosure may be co-administered with at least one additional therapeutic agent. In certain embodiments, an additional therapeutic agent is an anti-angiogenic agent. In certain embodiments, an additional therapeutic agent is a VEGF antagonist (in some embodiments, an anti-VEGF antibody, for example bevacizumab). In certain embodiments, an additional therapeutic agent is an EGFR antagonist (in some embodiment, erlotinib). In certain embodiments, an additional therapeutic agent is a chemotherapeutic agent and/or a cytostatic agent. In certain embodiments, an additional therapeutic agent is a taxoid (e.g., paclitaxel) and/or a platinum agent (e.g., carboplatinum). In certain embodiments the additional therapeutic agent is an agent that enhances the patient's immunity or immune system.

Such combination therapies noted above encompass combined administration (where two or more therapeutic agents are included in the same or separate formulations), and separate administration, in which case, administration of the antibody, antibody fragment or multi-specific antibody can occur prior to, simultaneously, and/or following, administration of the additional therapeutic agent and/or adjuvant. Antibodies or antibody fragments can also be used in combination with radiation therapy and/or surgical intervention.

The anti-EpCAM antibodies, antibody fragments or multi-specific antibodies may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody, antibody fragment or multi-specific antibody need not be, but is optionally formulated with one or more agents currently used to treat the disorder in question. The effective amount of such other agents depends on the amount of antibody, antibody fragment or multi-specific antibody present in the formulation, the type of disorder or treatment, and other factors discussed above. These are generally used in the same dosages and with administration routes as described herein or in any dosage and by any route that is empirically/clinically determined to be appropriate.

For the prevention or treatment of disease, the appropriate dosage of an antibody, antibody fragment or multi-specific antibody (when used alone or in combination with one or more other additional therapeutic agents) will depend on the type of disease to be treated, the type of antibody, antibody fragment or multi-specific antibody, the severity and course of the disease, whether the antibody or antibody fragment is administered for preventive or therapeutic purposes, previous therapy, the patient's clinical history and response to the antibody, antibody fragment or multi-specific antibody, and the discretion of the attending physician. The antibody or antibody fragment is suitably administered to the patient at one time or over a series of treatments. Depending on the type and severity of the disease, about 1 μg of antibody or antibody fragment/kg bodyweight of the patient to 40 mg of antibody or antibody fragment/kg bodyweight of the patient can be an initial candidate dosage for administration to the patient, whether, for example, by one or more separate administrations, or by continuous infusion. For repeated administrations over several days or longer, depending on the condition, the treatment would generally be sustained until a desired suppression of disease symptoms occurs. Such doses may be administered intermittently, e.g. every week or every three weeks (e.g. such that the patient receives from about two to about twenty, or e.g. about six doses of the antibody or antibody fragment). An initial higher loading dose, followed by one or more lower doses may be administered. However, other dosage regimens may be useful. The progress of this therapy is easily monitored by conventional techniques and assays.

Enhancing the host's immune function to combat tumors is the subject of increasing interest. Conventional methods include (i) APC enhancement, such as (a) injection into the tumor of DNA encoding foreign MHC alloantigens, or (b) transfecting biopsied tumor cells with genes that increase the probability of immune antigen recognition (e.g., immune stimulatory cytokines, GM-CSF, co-stimulatory molecules B7.1, B7.2) of the tumor, (iii) adoptive cellular immunotherapy, or treatment with activated tumor-specific T-cells. Adoptive cellular immunotherapy includes isolating tumor-infiltrating host T-lymphocytes, expanding the population in vitro, such as through stimulation by IL-2 or tumor or both. Additionally, isolated T-cells that are dysfunctional may be also be activated by in vitro application of the anti-PD-L1 antibodies of the disclosure. T-cells that are so-activated may then be readministered to the host. One or more of these methods may be used in combination with administration of an antibody, antibody fragment or multi-specific antibody of the present disclosure.

Traditional therapies for cancer include the following: (i) radiation therapy (e.g., radiotherapy, X-ray therapy, irradiation) or the use of ionizing radiation to kill cancer cells and shrink tumors. Radiation therapy can be administered either externally via external beam radiotherapy (EBRT) or internally via brachytherapy; (ii) chemotherapy, or the application of cytotoxic drug which generally affect rapidly dividing cells; (iii) targeted therapies, or agents which specifically affect the deregulated proteins of cancer cells (e.g., tyrosine kinase inhibitors imatinib, gefitinib; monoclonal antibodies, photodynamic therapy); (iv) immunotherapy, or enhancement of the host's immune response (e.g., vaccine); (v) hormonal therapy, or blockade of hormone (e.g., when tumor is hormone sensitive), (vi) angiogenesis inhibitor, or blockade of blood vessel formation and growth, and (vii) palliative care, or treatment directed to improving the quality of care to reduce pain, nausea, vomiting, diarrhea and hemorrhage. Pain medication such as morphine and oxycodone, anti-emetics such as ondansetron and aprepitant, can permit more aggressive treatment regimens.

In the treatment of cancer, any of the described conventional treatments for the treatment of cancer may be conducted, prior, subsequent or simultaneous with the administration of the anti-EpCAM antibodies, antibody fragments or multi-specific antibodies disclosed herein. Additionally, the anti-EpCAM antibodies, antibody fragments or multi-specific antibodies may be administered prior, subsequent or simultaneous with conventional cancer treatments, such as the administration of tumor-binding antibodies (e.g., monoclonal antibodies, toxin-conjugated monoclonal antibodies) and/or the administration of chemotherapeutic agents.

In another aspect of the disclosure, an article of manufacture containing an anti-EpCAM antibody, antibody fragment or multi-specific antibody and other materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody, antibody fragment or multi-specific antibody of the disclosure. The label or package insert indicates that the composition is used for treating the condition of choice. Moreover, the article of manufacture may comprise (a) a first container with a composition contained therein, wherein the composition comprises an anti-EpCAM antibody, antibody fragment or multi-specific antibody; and (b) a second container with a composition contained therein, wherein the composition comprises a further cytotoxic or otherwise therapeutic agent. The article of manufacture in this embodiment of the disclosure may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The disclosure also provides kits comprising at least one antibody, antibody fragment or multi-specific antibody of the disclosure. Kits containing antibodies, or antibody fragments, or multi-specific antibodies of the disclosure find use in detecting EpCAM expression (increase or decrease), or in therapeutic or diagnostic assays. Kits of the disclosure can contain an antibody coupled to a solid support, e.g., a tissue culture plate or beads (e.g., sepharose beads). Kits can be provided which contain antibodies for detection and quantification of EpCAM in vitro, e.g. in an ELISA or a Western blot. Such antibody useful for detection may be provided with a label such as a fluorescent or radiolabel.

The kits may further contain instructions on the use thereof. In some embodiments, the instructions comprise instructions required by the U.S. Food and Drug Administration or other applicable agency for in vitro diagnostic kits. In some embodiments, the kits comprise one or more antibodies, antibody fragments or multi-specific antibodies. In other embodiments, the kits further comprise one or more enzymes, enzyme inhibitors or enzyme activators. In still other embodiments, the kits further comprise one or more chromatographic compounds. In yet other embodiments, the kits further comprise one or more compounds used to prepare the sample for spectroscopic assay. In further embodiments, the kits further comprise comparative reference material to interpret the presence or absence of EpCAM according to intensity, color spectrum, or other physical attributes of an indicator.

The following examples are illustrative, but not limiting, of the anti-EPCAM antibodies of the present disclosure. Other suitable modifications and adaptations of the variety of conditions and parameters normally encountered in the field, and which are obvious to those skilled in the art, are within the scope of the disclosure.

### EXAMPLES

Examples 1-15 for making conditionally active antibodies are described in WO 2017/078839.

For Examples 16-28, an exemplary antibody (BA3182 or BAP150.31-BF45) comprising SEQ ID NOS: 98 and 99 was utilized.

| Light Chain Variable Region of | Heavy Chain Variable Region of |
|---|---|
| Anti-EPCAM of BA3182 | Anti-EPCAM of BA3182 |
| DIQMTQSPSS LSASVGDRVT | EVQLVESGGG LVQPGGSLRL |
| ITCSASSSIS YMHWYQQKPG | SCAASGYTFT SYWMHWIRQS |
| QAPRLLIYST SNLASGVPSR | PSRGLEWLGY IRPSTGYTEY |
| FSGSGSGTDF TFTISSLEAE | NQKFKDRFTI SRDDSKNTAY |
| DAATYYCHQW STYHTFGQGT | LQMNSLKTED TAVYYCGRGD |
| KVEIKRTVAA PSVFIFPPSD | NWVGFKNWGQ GTLVTVSSAS |
| EQLKSGTASV VCLLNNFYPR | TKGPSVFPLA PSSKSTSGGT |
| EAKVQWKVDN ALQSGNSQES | AALGCLVKDY FPEPVTVSWN |
| VTEQDSKDST YSLSSTLTLS | SGALTSGVHT FPAVLQSSGL |
| KADYEKHKVY ACEVTHQGLS | YSLSSVVTVP SSSLGTQTYI |
| SPVTKSFNRG ECSRSGGGGE | CNVNHKPSNT KVDKKVEPKS |
| VQLVESGGGL VQPGGSLRLS | CDKTHTCPPC PAPELLGGPS |
| CAASGFTENT YAMNWVRQAP | VFLFPPKPKD TLMISRTPEV |
| GKGLEWVARI RSKYNNYATY | TCVVVDVSHE DPEVKFNWYV |
| YADSVKDRFT ISRDDSKNSL | DGVEVHNAKT KPREEQYQST |
| CAASGFTFNT YAMNWVRQAP | YRVVSVLTVL HQDWLNGKEY |
| GKGLEWVARI RSKYNNYATY | KCKVSNKALP APIEKTISKA |
| YADSVKDRFT ISRDDSKNSL | KGQPREPQVY TLPPSRDELT |
| YLQMNSLKTE DTAVYYCVRH | KNQVSLTCLV KGFYPSDIAV |
| SNFGNSKVSW FAYWGQGTLV | EWESNGQPEN NYKTTPPVLD |
| TVSSGGSGGS GGSGGSGGQA | SDGSFFLYSK LTVDKSRWQQ |
| VVTQEPSLTV SPGGTVTLTC | GNVFSCSVMH EALHNHYTQK (SEQ ID |
| RSSAGAVTTS NYDNWVQQKP | NO: 99) |
| GQAPRGLIGG TNKRAPWTPA | |
| RFSGSLLGGK AALTITGAQA | |
| EDEADYYCAL WYSNLWVFGG | |
| GTKLTVLSR (SEQ ID NO: 98) | |

### Example 16. Affinity ELISA

This example shows the binding activity of BA3182 bispecific antibody to recombinant human CD3 and human EpCAM or cyno EpCAM extracellular domain (ECD) using an enzyme linked immunosorbent assay (ELISA). The ELISA assay was conducted with buffers at pH 6.0 (tumor microenvironment pH) or pH 7.4 (normal physiological pH). Serially diluted BA3182 was added to wells coated with recombinant CD3 epsilon and delta heterodimer extracellular domain. Bound BA3182 was quantified using human or cyno EpCAM extracellular domain fused to mouse Fc and anti-mouse IgG antibody conjugated to horseradish peroxidase (HRP), which reacted with 3,3′,5,5′ tetramethylbenzidine (TMB) colorimetric substrate to generate a colored product. The absorbance at 450 nm (OD₄₅₀) in each well is proportional to the amount of BA3182 bound to both human CD3 complex and human or cyno EpCAM with a dynamic range of 0.0847 to 50000 pM. EC50 values at different pH values for binding of BA3182 to human CD3 and human or cyno EpCAM were calculated using the nonlinear fit model (variable slope, four parameters) built into GraphPad Prism™ software. The EC50 values for BA3182 binding activity to human CD3 and human EpCAM at pH 6.0 were 297.83 pM, and 4155.67 pM at pH 7.4. The EC50 values for BA3182 binding activity to human CD3 and cyno EpCAM at pH 6.0 were 543.47 pM, and 29415.33 pM at pH 7.4. The results demonstrated that the binding activity of BA3182 to CD3 and/or EpCAM at normal physiological pH (pH 7.4) was significantly weaker than binding at the tumor microenvironment pH (pH 6.0).

### Materials

BA3182, 1.03 mg/mL

### Antigens:

Recombinant human CD3 epsilon and delta heterodimer, BioVision, Catalog #P11830-500,
lot number 7C19P11830.
Recombinant human EpCAM fused to mouse Fc (human EpCAM-mFec), Evitria, lot number 12919-SEC.
Recombinant cyno EpCAM fused to mouse Fc (cyno EpCAM-mFc), BioAtla, lot number 20072.

### Antibodies:

Goat anti-mouse HRP antibody, Promega, Catalog #W402B, lot number 0000465785.

### Reagents:

Carbonate-Bicarbonate buffer capsule, Sigma Catalog #C3041-100CAP, lot number SLBZ3401.
10×PBS, Gibco, Catalog #70011-044, lot number 2323767.
Albumin, Bovine (BSA), Sigma Aldrich, Catalog #A9647-100G, lot number SLCH8436.
Sodium bicarbonate (7.5% solution), Gibco, Catalog #25082-094, lot number 2336825. Tween-20, Sigma Aldrich, Catalog #P9416-50ML, lot number SLCJ0231.
TMB chromogen solution, Life Technology, Catalog #002023, lot number 05123211-7. 12N HCl: VWR, Cat #87003-251, lot #4118020.
ELISA assay plates, Thermo Scientific Nunc, Catalog #269787, lot number 1223409. Carbonate-Bicarbonate coating buffer: dissolve content of one capsule of Carbonate-Bicarbonate buffer in 100 mL of sterile water.
PBS buffer: dilute the 10×PBS buffer with distilled water to 1×.
pH ELISA incubation buffer: add sodium bicarbonate and BSA to the PBS buffer to have 2.5 g/L final sodium bicarbonate and 1% BSA, adjust pH to 6.0 or 7.4 using 1N HCl.
pH Wash buffer: add sodium bicarbonate and Tween-20 to PBS buffer to have 2.5 g/L final sodium bicarbonate and 0.1% Tween-20, adjust pH to 6.0 or 7.4 using 1N HCl. Stop solution: 1N HCl

Methods A 3-fold serial dilution of BA3182 in incubation buffer at pH 6.0 and pH 7.4 was carried out. The starting concentration of BA3182 for the complex hCD3/hEpCAM-mFc was 50 nM and the starting concentration of BA3182 for the complex hCD3/cyno EpCAM-mFc 150 nM.

### ELISA Assay

Coat ELISA plates with 100 μL/well of 1 μg/mL of hCD3 antigen in carbonate-bicarbonate coating buffer.
Cover plates with sealing film and incubate overnight at 4° C.
Decant plates and tap out residual liquid on a stack of paper towels.
Wash wells twice by dispensing 200 μL of incubation buffers at pH 6.0 or pH 7.4 to the wells and completely aspirate the contents.
Add 200 μL of incubation buffers at pH 6.0 or pH 7.4 to the wells. Cover with sealing film and place the plate onto a plate shaker set to 200 rpm for 60 minutes at room temperature. Decant plates and tap out residual liquid on a stack of paper towels.
Prepare 3-fold serial dilution of BA3182 starting at 50 nM or 150 nM in incubation buffer at pH 6.0 or pH 7.4.
Add duplicates of 100 μL/well of diluted BA3182 to the plates.
Cover with sealing film and place the plates onto a plate shaker (set to 200 rpm) for 60 minutes at room temperature.
Decant plates and tap out residual liquid on a stack of paper towels.
Wash wells three times by dispensing 200 μL of wash buffers at pH 6.0 or pH 7.4 to the wells and completely aspirate the contents.
Dilute human EpCAM-mFc in incubation buffers at pH 6.0 or pH 7.4 to 1 μg/mL.
Dilute cyno EpCAM-mFc in incubation buffers at pH 6.0 or pH 7.4 to 2 μg/mL.
Add 100 μL/well of the above diluted 1 μg/mL human EpCAM-mFc or 2 μg/mL cyno EpCAM-mFc to each well.
Cover with sealing film and place the plates onto a plate shaker set to 200 rpm for 60 minutes at room temperature.
Decant plates and tap out residual liquid on a stack of paper towels.
Wash wells three times by dispensing 200 μL of wash buffers at pH 6.0 or pH 7.4 pH to the wells and completely aspirate the contents.
Dilute the goat anti-mouse HRP secondary antibody at 1:2500 in incubation buffer at pH 6.0 or pH 7.4.
Add 100 μL of the above diluted goat anti-mouse HRP secondary antibody to each well. Cover with sealing film and place the plates onto a plate shaker (set to 200 rpm) for 60 minutes at room temperature.
Decant plates and tap out residual liquid on a stack of paper towels.
Wash wells three times by dispensing 200 μL of wash buffers at pH 6.0 or pH 7.4 to the wells and completely aspirate the contents.
Dispense 50 μL per well of the TMB substrate solution into all wells of the plates. For wells with human EpCAM-mFc, incubate at room temperature for 5 minutes. For wells with cyno EpCAM-mFc incubate for 10 minutes.
Add 50 μL/well of 1N HCl into all wells of the plates. Read plates at 450 nm using Molecular Device SpectraMax i3X microplate reader.
The average EC50 values of the binding activity of BA3182 to human CD3/human EpCAM-mFc or cyno EpCAM-mFec were calculated using the nonlinear fit model (variable slope, four parameters) built into GraphPad Prism software version 9.2.0.

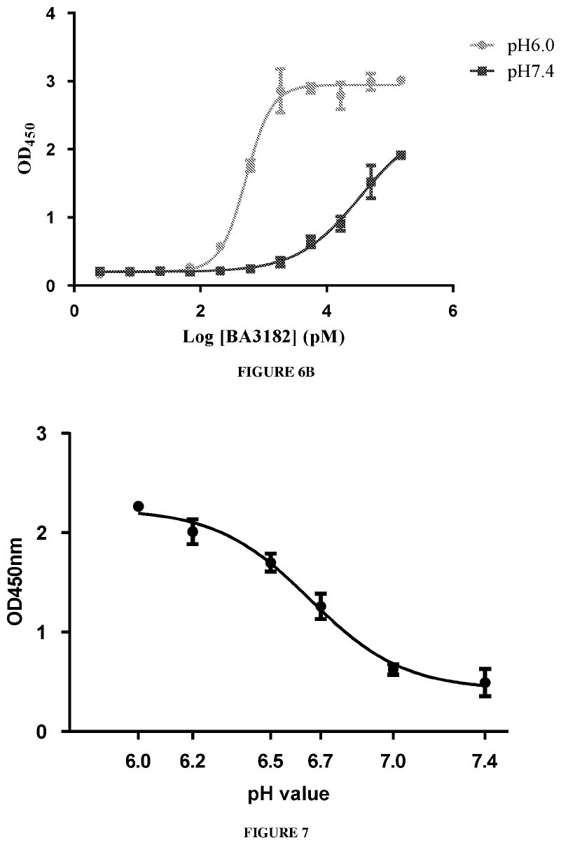

Results A total of three independent ELISA experiments were performed. A summary of the EC50 values of BA3182 to recombinant human CD3/human EpCAM-mFc and human CD3/cyno EpCAM-mFc at pH 6.0 and pH 7.4 are shown in Tables 3 & 4. The binding curves for the representative experiment are shown in FIGS. 6A and 6B. EC50 values of BA3182 at pH 6.0 measured by ELISA were 297.8 pM for human CD3/human EpCAM-mFc and 543.37 pM for human CD3/cyno EpCAM-mFc. At pH 7.4, BA3182 EC50s were 4155.67 pM for human CD3/human EpCAM-mFc and 29415.33 pM for human CD3/cyno EpCAM-mFc. Based on these results, it was observed that BA3182 bound to CD3 and EpCAM antigens with higher affinity at the tumor microevironment pH (pH 6.0) and with much lower affinity under physiological pH (pH 7.4).

TABLE 3
Binding Affinity of BA3182 to human CD3/human
EpCAM-mFc at pH 6.0 and pH 7.4.
pH affinity ELISA EC50 (pM)-hCD3/hEpCAM-mFc

| BA3182 | pH 6.0 | pH 7.4 |
|---|---|---|
| Experiment -1 | 299.30 | 4099.00 |
| Experiment -2 | 295.20 | 4375.00 |
| Experiment -3 | 299.00 | 3993.00 |
| Mean ± SD | 297.83 ± 2.29 | 4155.67 ± 197.20 |

TABLE 4
Binding Affinity of BA3182 to human CD3/cyno
EpCAM-mFc at pH 6.0 and pH 7.4.
pH affinity ELISA EC50 (pM)-hCD3/cynoEpCAM-mFc

| BA3182 | pH 6.0 | pH 7.4 |
|---|---|---|
| Experiment -1 | 528.00 | 33563.00 |
| Experiment -2 | 465.70 | 31633.00 |
| Experiment -3 | 636.70 | 23050.00 |
| Mean ± SD | 543.47 ± 86.54 | 29415.33 ± 5596.37 |

### Example 17. pH Range ELISA

This example shows the binding activity of BA3182 bispecific antibody to recombinant human CD3 and to human EpCAM extracellular domain in a range of pH buffers (pH 6.0 to pH 7.4), mimicking tumor microenvironment pH (pH 6.0 to pH 6.7) and the normal physiological pH (pH 7.4). Binding activity was measured using a sandwich enzyme linked immunosorbent assay (ELISA). Serially diluted BA3182 was bound to recombinant human CD3 epsilon/delta heterodimer extra cellular domain immobilized in the wells. The amount of bound BA3182 was quantified using human EpCAM extracellular domain fused to mouse Fc and anti-mouse IgG antibody conjugated to horseradish peroxidase (HRP), which then reacted with 3,3′,5,5′ tetramethylbenzidine (TMB) colorimetric substrate to generate a colored product. The absorbance at 450 nm (OD₄₅₀) in each well is proportional to the amount of BA3182 bound to both human CD3 complex and human EpCAM in the different pH buffers. Data analysis indicated the pH inflection point (=50% binding activity compared to pH 6.0) for BA3182 is at pH 6.66 with 90% of the binding activities occurring at pH 6.25 (tumor microenvironment pH). Significantly weaker binding activity of BA3182 was detected at pH 7.4 (normal physiological pH).

### Materials

### Antigens:

a) Recombinant human CD3 epsilon and delta heterodimer, BioVision Cat #P1183-500, lot number 7C19P11830.
b) Recombinant human EpCAM fused to mouse Fc (EpCAM-mFc), Evitria, lot number 12919-SEC.
   Antigen coating buffer, distilled water, Millipore.
   Goat anti-mouse HRP antibody, Promega, Cat #W402B, lot number 0000465785.
   Sodium bicarbonate, Sigma, Cat #55761-500G, lot number BCCD6088.
   PBS (1×): Cellgro, Cat #R21-040-CV, lot number 17321021.
   Tween-20: Sigma, Cat #P1379-500ML, lot number SLBS7482.
   Albumin, Bovine (BSA), VWR, Cat #0332, lot number 20D0656194.
   TMB chromogen solution, Life Technology, Cat #002023, lot number 08228211-7.
   HCl, Titansci, Cat #G81788B, lot number P1972715.
   ELISA assay plates, Corning, Cat #42592, lot number 00821030.
   PBS buffer: 0.144 g/L KH₂PO₄, NaCl 9 g/L, 0.795 g/L Na₂HPO₄ pH to 7.4
   pH ELISA Incubation buffer, pH 6.0, 6.2, 6.5, 6.7, 7.0 and 7.4: add 0.1 g sodium bicarbonate and 0.4 g BSA to 40 mL of 1×PBS buffer. Adjust pH to 6.0, 6.2, 6.5, 6.7, 7.0 and 7.4 using 1N HCl
   pH ELISA Wash buffer pH 6.0, 6.2, 6.5, 6.7, 7.0 and 7.4: add 0.15 g sodium bicarbonate, 0.6 g BSA to 60 mL and 0.1% Tween-20 of 1×PBS buffer. Adjust pH to 6.0, 6.2, 6.5, 6.7, 7.0 and 7.4 using 1N HCl
   Stop solution: 1N HCl, add 83.3 mL to 1 L distilled water

### Methods

EA1 was first diluted to 100 nM, and then diluted to 1.5 nM in various pH incubation buffer.
Coat ELISA plates with 100 μL of 1 μg/mL recombinant CD3 epsilon and delta complex antigen in distilled water.
Cover plates with sealing film and incubate overnight at 4° C.
Decant plates and tap out residual liquid on a stack of paper towels.
Wash wells twice by dispensing 200 μL of various pH incubation buffer to the wells and completely aspirate the contents.
Add 200 μL of various pH incubation buffer to the wells, cover with sealing film and place the plate onto a plate shaker (set to 200 rpm) for 60 minutes at room temperature.
Decant plates and tap out residual liquid on a stack of paper towels.
Serially dilute test substances in various pH incubation buffers to 1.5 nM.
Add 100 μL/well of BA3182 to the plates.
Cover with sealing film and place the plates onto a plate shaker (set to 200 rpm) for 60 minutes at room temperature.
Decant plates and tap out residual liquid on a stack of paper towels.
Wash wells three times by dispensing 200 μL of various pH wash buffers to the wells and completely aspirate the contents.
Dilute human EpCAM-mFc in various pH incubation buffers to 1 μg/mL.
Add 100 μL/well of 1 μg/mL human EpCAM-mFc diluted in various pH incubation buffers to each well.
Cover with sealing film and place the plates onto a plate shaker (set to 200 rpm) for 60 minutes at room temperature.
Decant plates and tap out residual liquid on a stack of paper towels.
Wash wells three times by dispensing 200 μL of various pH wash buffers to the wells and completely aspirate the contents.
Dilute the anti-mouse IgG HRP secondary antibody at 1:2500 in various pH incubation buffers.
Add 100 μL anti-mouse IgG HRP secondary antibody diluted in various pH incubation buffers to wells.
Cover with sealing film and place the plates onto a plate shaker (set to 200 rpm) for 60 minutes at room temperature.
Decant plates and tap out residual liquid on a stack of paper towels.
Wash wells three times by dispensing 200 μL of various pH wash buffer to the wells and completely aspirate the contents.
Dispense 5 μL per well of the TMB substrate solution into all wells of the plates. Incubate at room temperature for 5 minutes.
Add 50 μL per well of 1N HCl into all wells of the plates. Read plates at 450 nm using Molecular Device SpectraMax 190 microplate reader.

Data Analysis. Average OD values (from 2 replicates) were plotted against the different pH points tested using GraphPad Prism software. A curve fitting of 4-parameter with variable slope was used to calculate EC50 and EC90. The inflection point of the pH curve where 50% binding activity is observed, equals the EC50 of the fitting equation. Binding activity at pH 6.0 was set to 100%. The pH for 90% binding activity was interpolated from the fitted curve using non-linear regression of finding ECanything with the parameter set to EC90.

Results A summary of the binding activity of BA3182 to recombinant human CD3 and human EpCAM in various pH buffers is shown in Table 5. The binding curves for representative experiments are shown in FIG. 7. The pH inflection point for the binding activity of BA3182 was pH 6.66. The pH inflection point is the pH where 50% of the pH dependent binding of BA3182 was observed. 90% of the binding activity of BA3182 was reached at pH 6.25 (tumor microenvironment pH). In addition, weaker binding activities for BA3182 were detected at normal physiological pH (pH 7.4) (FIG. 7).

TABLE 5
pH inflection point (EC50) and EC90 values of the pH dependent
binding activities of BA3182 to human CD3/human EpCAM-mFc

| | Molecule |
|---|---|
| | BA3182 |
| | Method |
| | pH ELISA |
| | pH inflection point (EC50) |
| Experiment-1 | 6.706 |
| Experiment-2 | 6.656 |
| Experiment-3 | 6.619 |
| Mean ± SD | 6.660 ± 0.043 |
| | pH value (EC90) |
| Experiment-1 | 6.356 |
| Experiment-2 | 6.254 |
| Experiment-3 | 6.149 |
| Mean ± SD | 6.253 ± 0.104 |

### Example 18. Cross Species Affinity ELISA

This example shows the binding activity of BA3182 bispecific antibody to recombinant human CD3 and to human, cynomologus monkey (cyno), rat, and mouse EpCAM extracellular domains. The binding activity of BA3182 to human CD3 and EpCAM antigens from different species was evaluated using a sandwich enzyme linked immunosorbent assay (ELISA). The ELISA assay was carried out using assay buffers at pH 6.0 (tumor microenvironment pH) and at pH 7.4 (normal physiological pH). BA3182 at a concentration of 1.5 nM was added to wells containing immobilized recombinant human CD3 epsilon/delta heterodimer extra cellular domain. The amounts of bound BA3182 were quantified using human, cyno, rat or mouse EpCAM extracellular domains fused to mouse Fc (EpCAM-mFc) and anti-mouse IgG antibody conjugated to horseradish peroxidase (HRP), which then reacted with TMB colorimetric substrate to generate a colored product. The absorbance at 450 nm (OD₄₅₀) in each well was proportional to the amount of BA3182 bound to both human CD3 complex and EpCAM antigen. BA3182 shows strong binding activity to human CD3 complex and to human or cyno EpCAM at pH 6.0 but weaker binding activity at pH 7.4. For rat and mouse EpCAM, very low or no binding activity of BA3182 was observed at both pH values.

### Materials

### Antibody: BA3182

### Antigens:

a) Recombinant human CD3 epsilon and delta heterodimer, BioVision, Cat #P1183-500, lot number 7C19P11830.
b) Recombinant human EpCAM fused to mouse Fc (human EpCAM-mFc), Evitria, lot number 12919-SEC.
c) Recombinant cyno EpCAM fused to mouse Fc (cyno EpCAM-mFc), BioAtla, lot number 22007.
d) Recombinant rat EpCAM fused to mouse Fc (rat EpCAM-mFc), BioAtla, lot number 22009.
e) Recombinant mouse EpCAM fused to mouse Fc (mouse EpCAM-mFc), BioAtla, lot number 22011.
   Antigen coating buffer, distilled water, Millipore.
   Goat anti-mouse HRP antibody, Promega, Cat #W402B, lot number 0000465785.
   PBS (1×), Cellgro, Cat #R21-040-CV, lot number 17321021.
   Albumin, Bovine (BSA), VWR Cat #0332, lot number 20D0656194.
   Sodium bicarbonate, Sigma Cat #S5761-500G, lot number BCCD6088.
   Tween-20, Sigma Cat #P1379-500ML, lot number SLBS7482.
   TMB chromogen solution, Life Technology Cat #002023, lot number 08228211-7.
   HCl, Titansci Cat #G81788B, lot number P1972715.
   ELISA assay plates, Corning Cat #42592, lot number 01919010.
   pH meter, Alalis pH400.

### Shaker, Kylin-Bell TS-2.

Plate reader, Thermo Fisher Multiskan™ Sky 51119770DP, SN: 1530-800210C.
PBS buffer: 0.144 g/L KH₂PO₄, NaCl 9 g/L, 0.795 g/L Na₂HPO₄ pH 7.4.
pH ELISA Incubation buffer: add 0.5 g sodium bicarbonate and 2 g BSA to 200 mL of 1×PBS buffer. Adjust pH to 6.0 or 7.4 using 1N HCl.
pH ELISA Wash buffer: add 0.5 g sodium bicarbonate and 0.1% Tween-20 to 200 mL of 1×PBS buffer. Adjust pH to 6.0 or 7.4 using 1N HCl.
Stop solution: 1N HCl, add 83.3 ml to 1 L distilled water.

### Methods

BA3182 first diluted to 100 nM in PBS, and then diluted to 1.5 nM in pH 6.0 and pH 7.4 incubation buffers.
Coat ELISA plates with 100 μL of 1 g/mL recombinant CD3 epsilon and delta complex antigen in distilled water.
Cover plates with sealing film and incubate overnight at 4° C.
Decant plates and tap out residual liquid on a stack of paper towels.
Wash wells twice by dispensing 200 μL of pH 6.0 or pH 7.4 incubation buffer to the wells and completely aspirate the contents.
Add 200 μL of pH 6.0 or pH 7.4 incubation buffer to the wells. Cover with sealing film and place the plate onto a plate shaker (set to 200 rpm) for 60 minutes at room temperature.
Decant plates and tap out residual liquid on a stack of paper towels.
Serially dilute test substances in pH 6.0 or pH 7.4 incubation buffer to 1.5 nM.
Add 100 μL/well of diluted test substances to the plates.
Cover with sealing film and place the plates onto a plate shaker (set to 200 rpm) for 60 minutes at room temperature.
Decant plates and tap out residual liquid on a stack of paper towels.
Wash wells three times by dispensing 200 μL of pH 6.0 or pH 7.4 wash buffer to the wells and completely aspirate the contents.
Dilute human, cyno, rat or mouse EpCAM-mFc in pH 6.0 or pH 7.4 incubation buffer to 1 μg/mL
Add 100 μL/well of 1 μg/mL human, cyno, rat or mouse EpCAM-mFc diluted in pH 6.0 or pH 7.4 incubation buffer to each well.
Cover with sealing film and place the plates onto a plate shaker (set to 200 rpm) for 60 minutes at room temperature.
Decant plates and tap out residual liquid on a stack of paper towels.
Wash wells three times by dispensing 200 μL of pH 6.0 or pH 7.4 wash buffer to the wells and completely aspirate the contents.
Dilute the anti-mouse IgG HRP secondary antibody at 1:2500 in pH 6.0 or pH 7.4 incubation buffer.
Add 10 μL anti-mouse IgG HRP secondary antibody diluted in various pH incubation buffers to each well.
Cover with sealing film and place the plates onto a plate shaker (set to 200 rpm) for 60 minutes at room temperature.
Decant plates and tap out residual liquid on a stack of paper towels.
Wash wells three times by dispensing 200 μL of pH 6.0 or pH 7.4 wash buffer to the wells and completely aspirate the contents.
Dispense 50 μL per well of the TMB substrate solution into all wells of plates. Incubate at room temperature for 5 minutes.
Add 50 μL per well of 1N HCl into all wells of the plates. Read plates at 450 nm using the microplate reader.

Results A total of three independent ELISA experiments were performed. A representative experiment demonstrating the binding activities of BA3182 to recombinant human CD3 and to human, cyno, rat or mouse EpCAM at pH 6.0 and pH 7.4 is shown in FIG. 8. BA3182 showed strong binding activity to human CD3 and to human or cyno EpCAM at the tumor environment pH (pH 6.0) but much less binding activity at pH 7.4. Weak or no binding of BA3182 to rat and mouse EpCAM, was observed at both pH values.

### Example 19. Specificity ELISA

This example shows the binding activity of BA3182 bispecific antibody to recombinant human CD3, human EpCAM, human trophoblast cell surface antigen 2 (Trop2) and to a non-related human antigen. Trop2 and EpCAM belong to the tumor-associated calcium signal transducer gene family. They share a high sequence similarity in the extracellular domain and transmembrane region. The undisclosed non-related human antigen has no sequence similarities to human EpCAM molecule. The binding activity of BA3182 to these antigens was evaluated by affinity and sandwich enzyme linked immunosorbent assay (ELISA). The assay was conducted using buffer at pH 6.0 (tumor microenvironment pH), or at pH 7.4 (normal physiological pH). BA3182 was captured by recombinant human EpCAM or Trop2 (both fused to a his tag) immobilized in the wells, followed by anti-human IgG antibody conjugated to horseradish peroxidase (HRP) detection in the affinity ELISA assays. In the sandwich ELISA assays, BA3182 was captured by a recombinant human CD3E/6 heterodimer extracellular domain immobilized in the wells, followed by human EpCAM, human Trop2, or the non-related antigen (all fused to mouse Fc) addition. The bound complexes were detected with anti-mouse IgG antibody conjugated to HRP, which reacted with 3,3′,5,5′ tetramethylbenzidine (TMB) colorimetric substrate to generate a colored product. The absorbance at 450 nm (OD₄₅₀) in each well is proportional to the amount of EpCAM, Trop2, or the non-related antigen protein. For human Trop2, very low or no binding was observed in either pH 6.0 or pH 7.4 buffer. An anti-human Trop2 specific antibody (affinity ELISA assay) or bispecific antibody (sandwich ELISA assay) were included as a positive controls to confirm the binding affinity to human Trop2 antigen regardless of the pH buffers. Similarly, no binding of BA3182 was observed to the non-related antigen. Therefore, the binding activity of BA3182 bispecific antibody to recombinant human EpCAM was confirmed to be target specific.

### Materials

### Antibody: BA3182

### Antigens:

a) Recombinant human CD3 epsilon and delta heterodimer, BioVision Cat #P1183-500, lot number 7C19P11830.
b) Recombinant human EpCAM fused to mouse Fc, Evitria, lot number 12919-SEC.
c) Recombinant human Trop2 fused to mouse Fc, BioAtla, lot number 21042.
d) Recombinant non-related human antigen fused to mouse Fc, BioAtla, lot number 21044.
e) Recombinant human EpCAM fused to his tag, BioAtla, lot number 21058.
f) Recombinant human Trop2 fused to his tag, BioAtla, lot number 19044.
   Positive control antibody for Trop2, BioAtla, lot number PB01.
   Positive control bispecific antibody for Trop2, BioAtla, lot number 860650.
   Positive control bispecific antibody for non-related human antigen, BioAtla, lot number 21-10458.
   Antigen coating buffer, distilled water, Millipore.
   Carbonate-Bicarbonate buffer capsule, Sigma Catalog #C3041-100CAP, lot number SLBZ3401.
   Goat anti-human IgG HRP antibody, Promega Cat #W403B, lot number 0000423844
   Goat anti-mouse IgG HRP antibody, Promega Cat #W402B, lot number 0000465785.
   PBS (1×): Cellgro, Cat #R21-040-CV, lot number 17321021.
   Albumin, Bovine (BSA), VWR Cat #0332, lot number 20D0656194.
   Sodium bicarbonate, Sigma Cat #55761-500G, lot number BCCD6088.
   Tween-20, Sigma Cat #P1379-500ML, lot number SLBS7482.
   TMB chromogen solution, Life Technology Cat #002023, lot number 08228211-7.
   HCl, Titansci Cat #G81788B, lot number P1972715.
   ELISA assay plates, Corning Cat #42592, lot number 01919010.
   pH meter, Alalis pH400.

### Shaker, Kylin-Bell TS-2.

Plate reader, Thermo Fisher Multiskan™ Sky 51119770DP, SN: 1530-800210C.
PBS buffer: 0.144 g/L KH₂PO₄, NaCl 9 g/L, 0.795 g/L Na₂HPO₄ pH7.4.
Carbonate-Bicarbonate coating buffer: dissolve content of one capsule of Carbonate-Bicarbonate buffer in 100 mL of sterile water.
pH ELISA Incubation buffer: Add 0.5 g sodium bicarbonate and 2 g BSA to 200 mL of 1×PBS buffer. Adjust pH to 6.0 or 7.4 using 1N HCl.
pH ELISA Wash buffer: Add 0.5 g sodium bicarbonate and 0.1% Tween-20 to 200 mL of 1×PBS buffer. Adjust pH to 6.0 or 7.4 using 1N HCl.
Stop solution: 1N HCl, add 83.3 mL to 1 L distilled water.

### Methods

BA3182 was first diluted to 100 nM in PBS, and then diluted to 2.5 nM for related family ELISA and 1.5 nM for non-related antigen ELISA in pH 6.0 or pH 7.4 incubation buffer. Coat affinity ELISA plates with 100 μL of 1 μg/mL recombinant human EpCAM or Trop2 antigen in carbonate-bicarbonate coating buffer.
Coat sandwich ELISA plate with 100 μL of 1 μg/mL human recombinant CD3 epsilon and delta complex antigen in distilled water.
Cover plates with sealing film and incubate overnight at 4° C.
Decant plates and tap out residual liquid on a stack of paper towels.
Wash wells twice by dispensing 200 μL of pH 6.0 or pH 7.4 incubation buffer to the wells and completely aspirate the contents.
Add 200 μL of pH 6.0 or pH 7.4 incubation buffer to the wells. Cover with sealing film and place the plate onto a plate shaker (set to 200 rpm) for 60 minutes at room temperature.
Decant plates and tap out residual liquid on a stack of paper towels.
Serially dilute test substances in pH6.0 or pH 7.4 incubation buffer to 2.5 nM or 1.5 nM or 0.5 nM.
Add 100 μL/well of diluted BA3182 to the plates.
Cover with sealing film and place the plates onto a plate shaker (set to 200 rpm) for 60 minutes at room temperature.
Decant plates and tap out residual liquid on a stack of paper towels.
Wash wells three times by dispensing 200 μL of pH 6.0 or pH 7.4 wash buffer to the wells and completely aspirate the contents.
For affinity ELISA, Dilute the anti-human IgG HRP secondary antibody at 1:2500 in pH 6.0 or pH 7.4 incubation buffer. Add 100 L anti-human IgG HRP secondary antibody diluted in various pH incubation buffers to each well.
For sandwich ELISA, dilute human EpCAM-mFc, human Trop2-mFc or non-related human antigen-mFc in pH 6.0 or pH 7.4 incubation buffer to 1 μg/mL or 2 μg/mL. Add 100 μL/well of the above diluted 1 μg/mL human EpCAM-mFc, 2 μg/mL human Trop2-mFc or non-related human antigen-mFc to each well.
Cover with sealing film and place the plates onto a plate shaker (set to 200 rpm) for 60 minutes at room temperature.
Decant plates and tap out residual liquid on a stack of paper towels.
Wash wells three times by dispensing 200 μL of pH 6.0 or pH 7.4 wash buffer to the wells and completely aspirate the contents.
For sandwich ELISA, dilute the anti-mouse IgG HRP secondary antibody at 1:2500 in pH 6.0 or pH 7.4 incubation buffer.
Add 100 μL anti-mouse IgG HRP secondary antibody diluted in various pH incubation buffers to each well.
Cover with sealing film and place the plates onto a plate shaker (set to 200 rpm) for 60 minutes at room temperature.
Decant plates and tap out residual liquid on a stack of paper towels.
Wash wells three times by dispensing 200 μL of pH 6.0 or pH 7.4 wash buffer to the wells and completely aspirate the contents.
Dispense 50 μL per well of the TMB substrate solution into all wells of plates. Incubate at room temperature for 5 minutes.
Add 50 μL per well of 1N HCl into all wells of the plates. Read plates at 450 nm using the microplate reader.

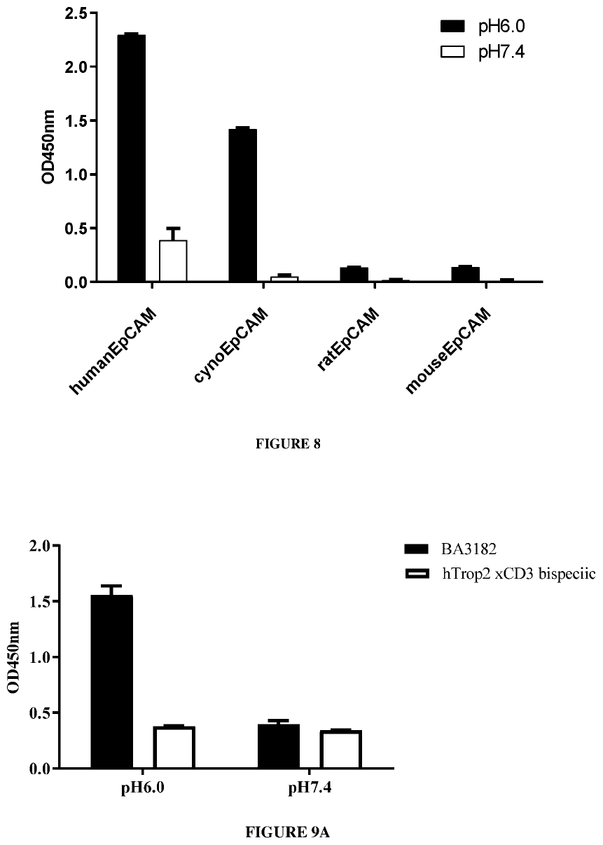

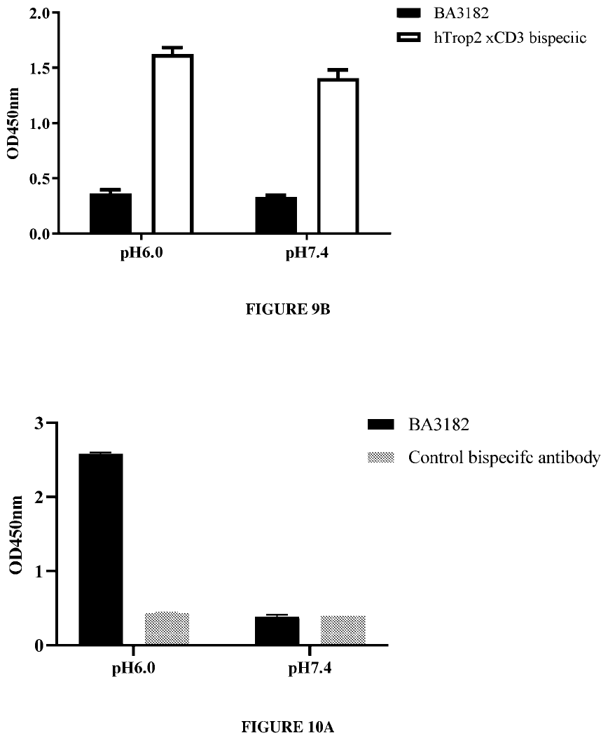

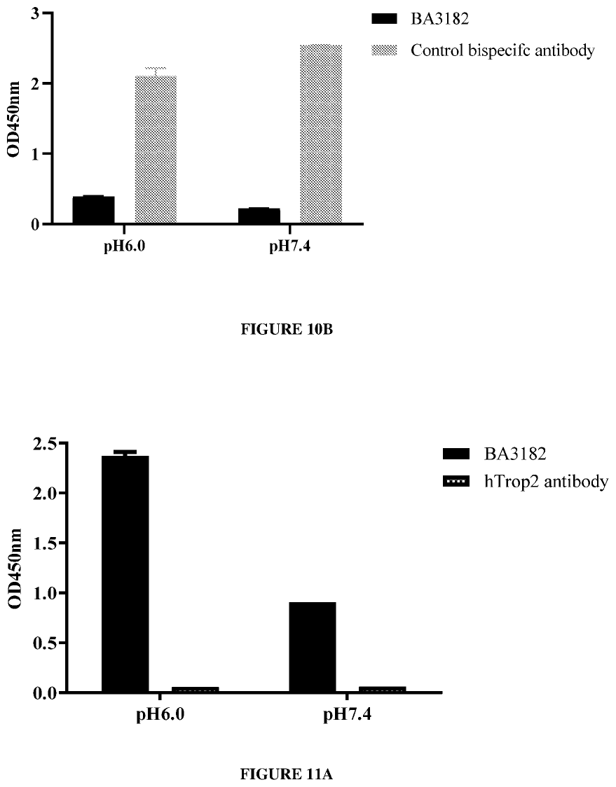

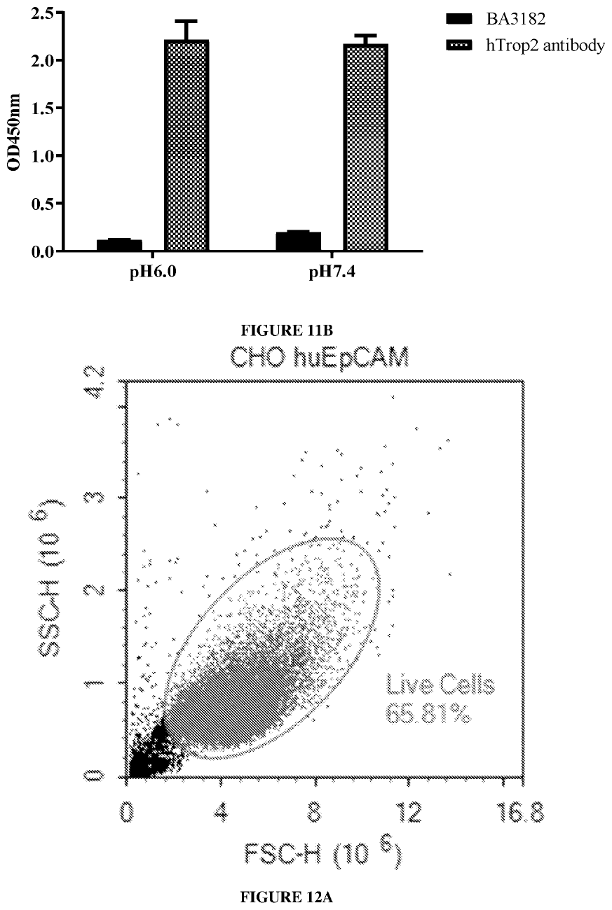

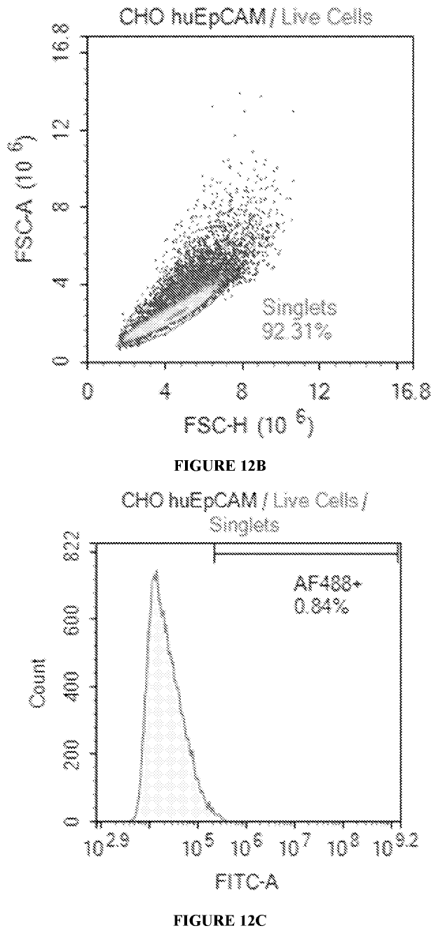

Results A total of three independent ELISA experiments with duplicate samples were performed. A representative experiment demonstrating the binding activities of BA3182 to recombinant human CD3 and human EpCAM or human Trop2 ECD at pH 6.0 and pH 7.4 is shown in FIGS. 9A and 9B. The binding activities of BA3182 to recombinant human CD3 and human EpCAM or non-related human antigen at pH 6.0 and pH 7.4 are shown in FIGS. 10A and 10B. The binding activities of BA3182 to recombinant human EpCAM or human Trop2 ECD at pH 6.0 and pH 7.4 are shown in FIGS. 11A and 11B.

BA3182 showed high binding activity to human EpCAM (FIG. 9A, FIG. 10A and FIG. 11A) at pH 6.0 and lower binding at pH 7.4. BA3182 showed no binding to human Trop2 (FIG. 9B and FIG. 11B) and to the non-related human antigen (FIG. 10B) at both, pH 6.0 and pH 7.4. The data show that BA3182 has high specificity for human EpCAM and CD3.

### Example 20. Binding of BA3182 to EpCAM or CD3 Expressing Cells Analyzed by FACS

This example shows the binding activity of BA3182 bispecific antibody to EpCAM or CD3 expressing cells. The binding activity of BA3182 to EpCAM expressing cells was evaluated using CHO cells expressing human EpCAM, CHO cells expressing cynoEpCAM and HCT116 cells. The binding activity of BA3182 to CD3 expressing cells was evaluated using Jurkat T cells and peripheral blood mononuclear cells (PBMCs) (human, cyno, rat, canine). The cells were incubated with various concentrations of BA3182 at pH 6.0 (tumor microenvironment pH) and at pH 7.4 (normal physiological pH). BA3182 bound to the cells was quantified using anti-human IgG antibody conjugated to Alexa Fluor 488 (AF488). Stained cells were analyzed by fluorescence-activated cell sorting (FACS) to determine the median of fluorescence intensity (MFI), which was proportional to the amount of BA3182 bound to the cells. At each pH, MFI values and corresponding BA3182 concentrations were analyzed using GraphPad Prism software. Non-linear four parameters' curves with variable slopes were used to determine the EC50 values of the binding activity of BA3182 to EpCAM and CD3 molecules expressed on the cell surface at both pH 6.0 and pH 7.4. The mean EC50 values of BA3182 bound to EpCAM and CD3 expressing cells are listed in Tables 6 and 7. Overall, at pH 6.0 BA3182 bound with high affinity to human and cynoEpCAM and CD3 antigens expressed on the cell surface. Decreased affinity of BA3182 to these molecules was observed at pH 7.4, especially to CD3 molecule. No binding of BA3182 was observed to rat, mouse and canine PBMCs. The homology between human, and rat, mouse or canine CD3 molecules is low (below 60%). This result suggests that the CD3 epitope of BA3182 is not present in the rat, mouse and canine CD3 antigens.

### Materials

BA3182 1.03 mg/mL.
Isotype control was produced by Evitria (Zurich, Switzerland), lot number #10229, 3 mg/mL.
CHO-hEpCAM: CHO-S cells, ThermoFisher Cat #R80007 transfected to stably express human EpCAM (synthesized and constructed at BioAtla), clone #32.
CHO-cynoEpCAM: CHO-S cells, Thermo Fisher Cat #R80007 transfected to stably express cynomolgus EPCAM (synthesized and constructed at BioAtla), clone #3.
HCT116, human colon carcinoma, ATCC, Cat #CCL-247™.
Jurkat, human T lymphocyte cells, ATCC, Cat #TIB-152, clone E6-1.

### Human PBMC: Precision for Medicine, Cat #39000, lot #201013292.

### Cyno PBMC: Worldwide Primates, Cat #CA-10, lot #C0767-22.

### SD Rat PBMC: Iqbiosciences, Cat #IQB-RPB101, lot #P20K0105.

Balb/c mouse PBMC: lqbiosciences, Cat #IQB-MPB101, lot #P21A0603.

### Beagle Canine PBMC: Iqbiosciences, Cat #IQB-CPB102, lot #P21C2303.

Mouse anti-hEpCAM conjugated to PE: BioLegend, Cat #324206, clone 9C4, lot #B222943.
Mouse IgG2b isotype conjugated to PE: BioLegend, Cat #400314, clone MPC-11, lot #B214529.
Goat anti-human IgG AF488 antibody: Thermo Fisher, Cat #A11013, lot #2110842.

### PBS: 10×, Thermo Fisher, Cat #14190-144, lot #2152877.

Cell culture grade water: Cytiva Cat #SH30529.03, lot #AH30009593.
Trypan blue stain: Thermo Fisher, Cat #15250-061, lot #1861515.

### BSA: Sigma, Cat #A9647, lot #SLBV4996.

### TrypLE™ Express Enzyme (1×): Thermo Fisher, Cat #12605-10, lot #2193025

### FBS: Thermo Fisher, Cat #16140-011, lot #2372673RP.

### McCoy's 5A: Thermo Fisher, Cat #16600-082, lot #232363.

### DMEM: Thermo Fisher, Cat #11965-084, lot #2346179.

### RPMI-1640: Thermo Fisher, Cat #11875-085, lot #2463433.

### 100×NEAA: Thermo Fisher, Cat #11140-050 lot #2337217.

100 mM Sodium pyruvate: Thermo Fisher, Cat #11360-070, lot #2323639.
G418: Invivogen, Cat #ant-gn-5, lot #GNL-38-06A.
Sodium bicarbonate: Thermo Fisher. Cat #25080-094, lot #2039755.

### 12N HCl: VWR, Cat #87003-251, lot #4118020.

T-150 tissue culture flask: Corning Cat #430825.
50 mL conical tube: BioPioneer Cat #CNT-50, lot #10272021.
96-well U-bottom plate: Greiner bio-one, Cat #650101, lot #B17033C7.
pH Meter: ORION STAR A111, Thermo Fisher, serial #J17162

### Shaker: MaxQ 2000.

### Flow Cytometer: ACEA NovoCyte 20602.

PBS buffer: add 100 mL 10×PBS buffer to 900 mL sterile water.
Wash buffer, pH 6.0: add 10 mL 75 g/mL sodium bicarbonate solution to 290 mL of 1×PBS buffer. Adjust pH to 6.0 using 6N or 0.6N HCl.
Wash buffer, pH 7.4: add 10 mL 75 g/mL sodium bicarbonate solution to 290 mL of 1×PBS buffer. Adjust pH to 7.4 using 6N or 0.6N HCl.
FACS buffer, pH 6.0: add 1.5 mL of 30% BSA to 43.5 mL of wash buffer. Adjust pH to 6.0 using 6N or 0.6N HCl.
FACS buffer, pH 7.4: add 1.5 mL of 30% BSA to 43.5 mL of wash buffer. Adjust pH to 7.4 using 6N or 0.6N HCl.
CHO-hEpCAM/CHO-cynoEpCAM culture media: DMEM+1×NEAA (0.1 mM non-essential amino acid solution)+1× sodium pyruvate (1 mM)+10% FBS+1 mg/mL G418.
HCT116 culture media: McCoy's 5A+10% FBS.
Jurkat culture media: RPMI-1640+10% FBS.

### Methods

Cell Culturing CHO-hEpCAM, CHO-cynoEpCAM, HCT116 and Jurkat cells were maintained in the designated culture media and were routinely sub-cultured twice a week. For FACS analysis, cells were harvested during the exponential growth phase. Frozen human, cyno, rat, mouse and canine PBMC were freshly thawed in RPMI-1640/10% FBS media and analyzed by FACS.

### Cell Staining

On the day of FACS analysis, remove and discard culture medium.
Briefly rinse the cell layer with PBS solution.
Add 3 mL of TrypLE™ Express enzyme solution to each of the T-150 flasks. Wait until cell layer is dispersed.
Add 7 mL of culture media to the flasks and resuspend cells by gentle pipetting.
Pool the cells and transfer the cell suspension to a 50 mL conical tube.
Count the cells with trypan blue staining before centrifugation at 2000 rpm for 3 min at 4° C.
Wash the cells once with PBS and transfer 3×10⁵ cells into an Eppendorf tube.
Add 2 μL of mouse anti-hEpCAM (PE conjugated mouse IgG) or PE-isotype mouse IgG to 100 μL of PBS solution with 1% BSA. Add 100 μL of the diluted PE antibody per tube and shake at 100 RPM for one hour on ice with protection from light.
Wash cells three times with 150 μL PBS solution.
Fix cells with 4% PFA for 10 min at R.T., then wash cells with PBS once.
Resuspend cells in 100 μL PBS and analyze the cells on NovoCyte flow cytometer.
BD Quantibrite™ PE beads standards were used to estimate the number of EpCAM molecules on the cell surface.

### Binding Analysis of BA3182 to EpCAM Expressed on CHO-hEpCAM, CHO-cynoEpCAM and HCT116 Cells

Harvest the cells as described in the Cell Staining section above and wash the cells once with PBS.
Adjust cell concentration to 3×10⁶ cells/mL in pH6.0 or pH7.4 wash buffer.
Aliquot 3×10⁵ cells in 100 μL pH6.0 or pH7.4 wash buffer into 96-well U-bottom plates.
Spin down the cells and discard the buffer.
Make 3-fold serial dilutions of the antibodies starting at 1500 nM for CHO-cynoEpCAM or 500 nM for CHO-hEpCAM and HCT116 in pH6.0 or pH7.4 FACS buffer.
Add 100 L/well of the diluted test article to cells, gently mix and incubate on ice with shaking (100 rpm) for one hour.
Centrifuge the cells at 2000 rpm for 3 min at 4° C. Wash the cells with 150 L of pH6.0 or pH7.4 wash buffer two times.
Dilute the goat anti-human IgG AF488 antibody 1:300 in pH6.0 or pH7.4 FACS buffer. Add 100 μL of the diluted antibody from the step above to the cells and incubate on ice for 45 minutes with protection from light.
Pellet the cells and wash with 150 μL of pH6.0 or pH7.4 wash buffer three times.
Fix cells with 4% paraformaldehyde (PFA) diluted in 1×PBS for 10 min at R.T., then wash cells with 1×PBS.
Resuspend the cells in 100 μL of 1×PBS.
Analyze the cells using NovoCyte Flow Cytometer and software. Acquire 20,000 events of each sample.

### Binding Analysis of BA3182 to CD3 Expressed on Jurkat T Cells, Human, Cyno, Rat, Mouse and Canine PBMCs.

Prepare the cells suspension as described in the Cell Culturing section above. Wash the cells once with PBS.
Adjust cell concentration to 3×10⁶/mL in pH 6.0 or pH 7.4 wash buffer.
Aliquot 3×10⁵ cells in 100 μL pH 6.0 or pH 7.4 wash buffer into 96-well U-bottom plates.
Spin down the cells and discard the buffer.
Make 3-fold serial dilutions of the antibodies starting at 2500 nM in pH 6.0 or pH 7.4 FACS buffer.
Add 100 μL/well of the diluted antibodies to the cells, gently mix and incubate on ice with shaking at 100 rpm for one hour.
Centrifuge the cells at 2000 rpm for 3 min at 4° C. Wash the cells with 150 μL of pH 6.0 or pH 7.4 wash buffer two times.
Dilute the goat anti-human IgG AF488 antibody at 1:300 in pH 6.0 or pH7.4 FACS buffers.
Add 100 μL of the diluted secondary antibody to the cells and incubate on ice for 45 minutes with protection from light.
Pellet the cells and wash with 150 μL of pH 6.0 or pH 7.4 wash buffer three times.
Fix cells with 4% PFA diluted in 1×PBS for 10 min at R.T. Wash cells with 1×PBS and centrifuge at 2000 rpm for 3 min at 4° C.
Resuspend the cells in 100 μL of 1×PBS. Analyze the cells using NovoCyte Flow Cytometer and software. Acquire 20,000 events of each sample.

Data Analysis Median of fluorescence intensity (MFI) and antibody concentrations were used to generate 4-parameter nonlinear regression curves with variable slope using GraphPad Prism software version 9.2.0.

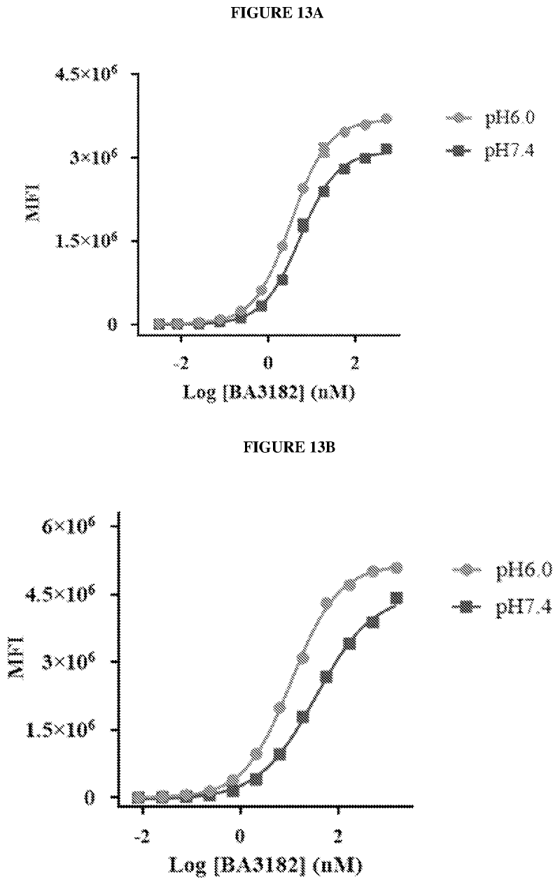

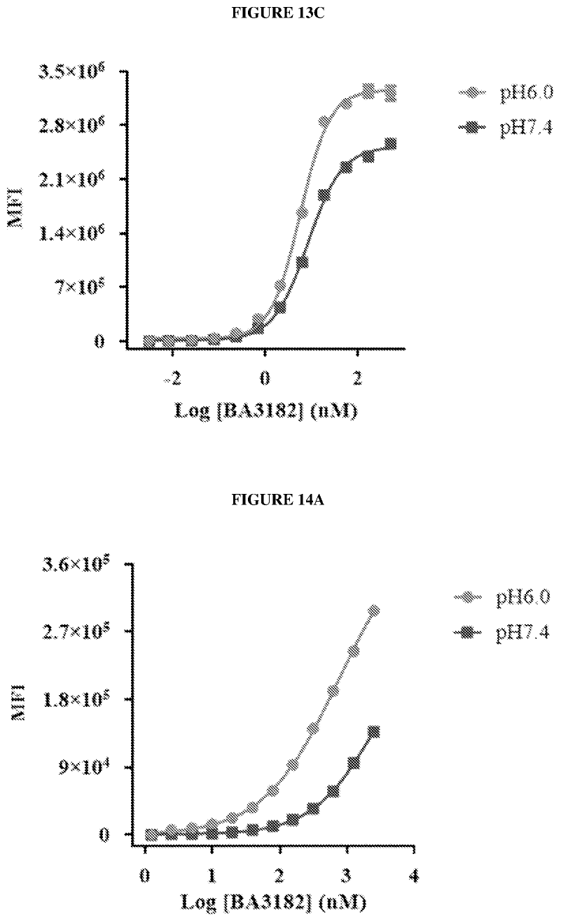

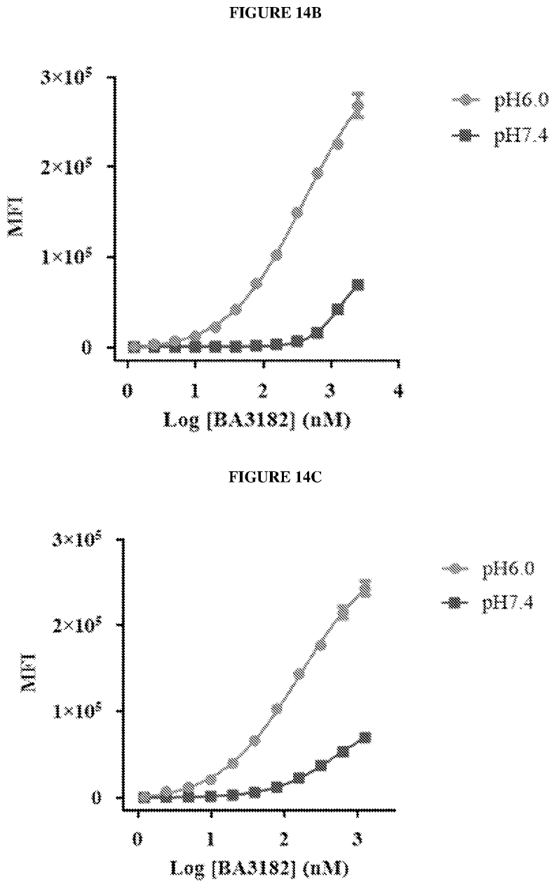

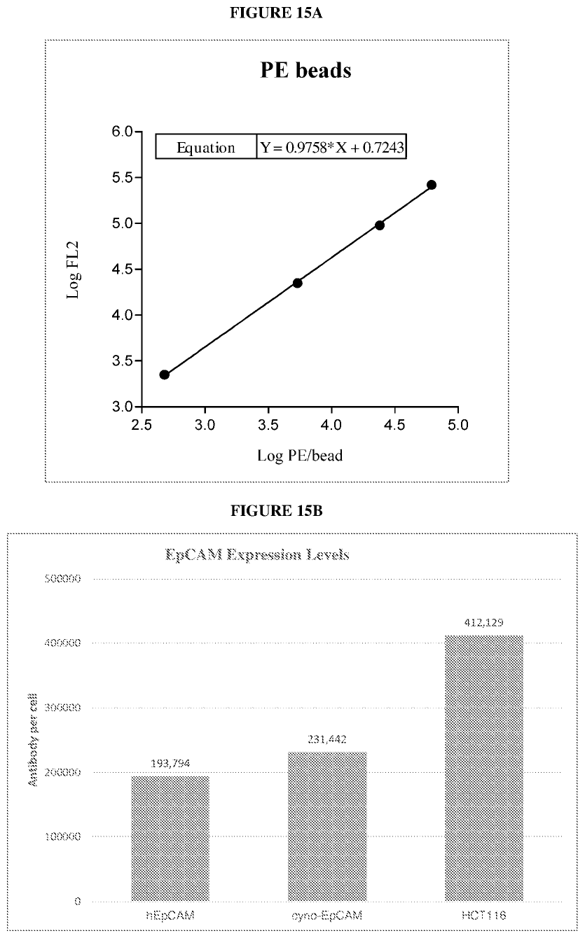

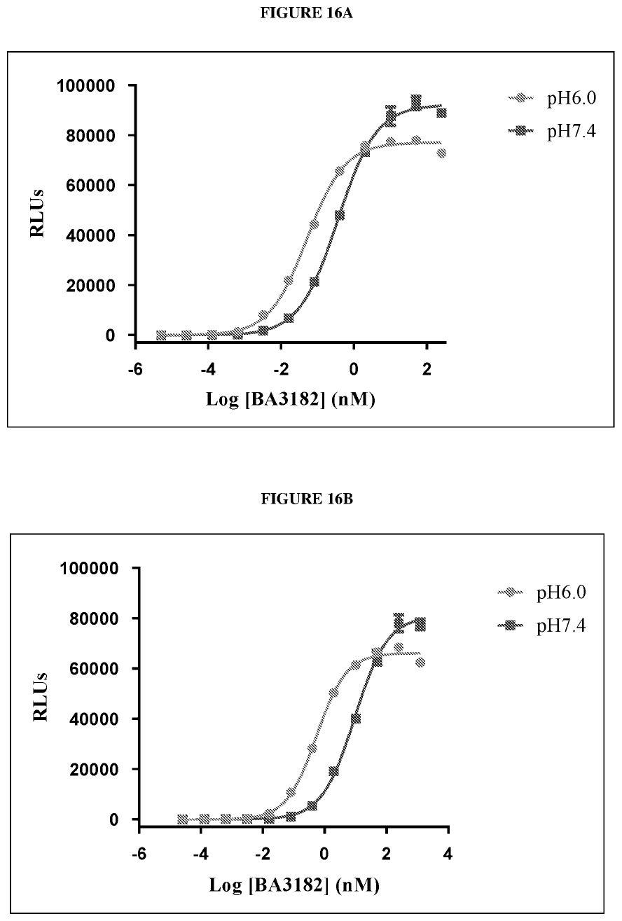

Results At least three independent FACS experiments were performed for each cell line or PBMC tested. The gating strategy for the determination of positive cells is shown in FIGS. 12A-12C. The binding activities of BA3182 to CHO-hEpCAM, CHO-cynoEpCAM, and HCT116 cells are shown in FIGS. 13A-13C. The binding activities of BA3182 to Jurkat cells, human and cyno PBMCs are shown in FIGS. 14A-14C. The expression levels of EpCAM molecules on the cell surface are shown in FIGS. 15A-15B.

BA3182 bound well to EpCAM expressing cells, generating good dose-response curves at pH 6.0 and pH 7.4 (FIGS. 13A-13C and 14A-14C). Mean EC50 values of BA3182 binding to EpCAM at tumor microenvironment pH (pH 6.0) were 3.4 nM, 11.0 nM and 5.9 nM for human and cyno EpCAM expressed in CHO cells, and for hEpCAM expressed on HCT116 cells, respectively (Table 6). The Mean EC50 values of BA3182 binding to EpCAM at normal physiological pH (pH 7.4) was 5.6 nM, 28.6 nM and 8.3 nM for human and cyno EpCAM expressed in CHO cells, and for hEpCAM expressed on HCT116 cells, respectively (Table 6). BA3182 bound with lower affinity to EpCAM antigen expressed on the cell surface at pH 7.4. No binding to naive CHO cells was observed (data not shown).

Dose-response curves of BA3182 binding to CD3 expressing cells is shown in FIGS. 14A-14C. BA3182 bound with higher affinity to CD3 antigen at pH 6.0. BA3182 bound to CD3 expressing cells with significantly lower affinity at pH 7.4. Mean EC50 values of BA3182 binding to CD3 expressed on the cell surface were 808.6 nM, 460.3 nM, and 161.6 nM for human PBMC, cyno PBMC, and Jurkat cells, respectively (Table 7). The mean EC50 values for the binding activities of BA3182 in physiological pH (pH 7.4) were 2337.3 nM, 1717.3 nM, and 518.2 nM for human PBMC, cyno PBMC, and Jurkat cells, respectively (Table 7). No binding of BA3182 to rat, mouse and canine PBMCs was observed (data not shown).

TABLE 6
Binding affinity of BA3182 EpCAM expressing cells.
BA3182 EC50 (nM) Mean ± SD

| Cell Type | pH 6.0 | pH 7.4 |
|---|---|---|
| CHO hEpCAM | 3.41 ± 0.85 | 5.65 ± 1.33 |
| CHO cynoEpCAM | 11.02 ± 0.73 | 28.55 ± 5.02 |
| HCT116 | 5.01 ± 0.31 | 8.28 ± 0.14 |

TABLE 7
Binding of BA3182 CD3 expressing cells.
BA3182 EC50 (nM) Mean ± SD

| Cell Type | pH 6.0 | pH 7.4 |
|---|---|---|
| Human PBMC | 808.60 ± 68.00 | 2337.33 ± 386.40 |
| Cyno PBMC | 460.30 ± 74.47 | 1717.33 ± 341.52 |
| Jurkat T cells | 161.60 ± 10.70 | 518.20 ± 15.90 |

### Example 21. Functional T Cell Activation Bioassay

This example shows the functional activity of BA3182 bispecific antibody using a Promega T cell activation bioassay. EpCAM expressing cell lines, HCT116, CHO cells expressing hEpCAM and CHO cells expressing cynoEpCAM were cultured in the presence of titrated concentrations of BA3182 and an isotype control bispecific antibody, followed by the addition of TCR/CD3 effector cells according to the vendor's protocol. TCR/CD3 effector cells, when engaged via BA3182 bispecific antibodies in the presence of EpCAM expressing cells, activate the NFAT pathway, resulting in an NFAT-RE-mediated luminescence signal that is detected by addition of Bio-Glo™ Reagent and quantified with a luminometer. To determine BA3182 EC50 values, the relative luminescent unit (RLU) values were analyzed using the nonlinear fit model (variable slope, four parameters) built into GraphPad Prism software. The mean EC50 of BA3182 added to CHO cells expressing hEpCAM was 0.049 nM at pH 6.0 and 0.383 nM at pH 7.4. The mean EC50 of BA3182 added to CHO cells expressing cynoEpCAM was 0.576 nM at pH 6.0 and 9.647 nM at pH 7.4. The mean EC50 of BA3182 added to HCT116 cells was 0.273 nM at pH 6.0 and 1.988 nM at pH 7.4. These results demonstrated that BA3182 was very potent in mediating TCR/CD3 engagement of T cells at pH 6.0 (tumor microenvironment) and significantly less potent at physiological pH (pH 7.4).

### Materials

BA3182
RPMI-1640: ThermoFisher Gibco Cat #11875-085, Lot #2463433
DMEM: ThermoFisher Gibco Cat #11965-084, Lot #2346179
FBS: ThermoFisher Gibco Cat #16140-011, Lot #2372673RP
100×NEAA: Gibco, Cat #11140-050 Lot #2337217
100 mM Sodium pyruvate: Gibco, Cat #11360-070, Lot #2323639
Bio-Glo™ buffer: Promega, Cat #G719A, Lot #0000473817
Bio-Glo™ assay substrate: Promega, Cat #G720A, Lot #0000481126
Cell culture grade water: Cytiva Cat #SH30529.03, Lot #AH30009593
12N HCl: VWR, Cat #87003-251, Lot #4118020
50 mL conical tube: BioPioneer Cat #CNT-50, Lot #10272021
96-well assay plate: Corning, Cat #3917, Lot #32021006
Syringe filter: Celltreat Cat #229747, Lot #211004-052-1A
pH Meter: ORION STAR A111, ThermoFisher, serial #J17162
Adhesive plate sealing films for microplate: E&K Scientific, Seal Plate, Cat #T396100 Lot #SG262G
Shaker: MaxQ 2000
Sample dilution block: Corning, Cat #3956, Lot #07718000
Plate reader: Molecular Devices, SpectraMax i3X
pH 6.0 assay buffer: DMEM+1×NEAA (0.1 mM non-essential amino acid solution)+1× sodium pyruvate (1 mM)+10% FBS, adjust to pH 6.0 using 6N HCl, sterilize with 0.2 μm Syringe filter.
pH 7.4 assay buffer: DMEM+1×NEAA (0.1 mM non-essential amino acid solution)+1× sodium pyruvate (1 mM)+10% FBS adjust to pH 7.4 using 6N HCl, sterilize with 0.2 m Syringe filter.
CHO-hEpCAM/CHO-cynoEpCAM culture media: DMEM+1×NEAA (0.1 mM non-essential amino acid solution)+1× sodium pyruvate (1 mM)+10% FBS+1 mg/mL G418
HCT116 culture media: McCoy's 5A+10% FBS
CHO hEpCAM clone #9, BioAtla, passage #11.
CHO cynoEpCAM clone #3, BioAtla, passage #13.
HCT116, human colon carcinoma, ATCC, Cat #CCL-247™, passage #10.
TCR/CD3 effector cells, Promega, T cell activation bioassay (NFAT), propagation model,
Cat #11601, expanded/cryopreserved, passage #7.

### Methods

Prepare a 2× working solution in pH 6.0 or pH 7.4 assay buffer of the test article, 500 nM for CHO hEpCAM and HCT116 cells, 2500 nM for cynoEpCAM cells. Then make a 5-fold serial dilution for a total of 11 data points.
Dispense 25 μL of serially diluted 2× test article solutions to the wells according to the plate layout. The final starting concentration is 250 nM or 1250 nM according to the target cells.
One day before the assay, seed EpCAM expressing cells into a 96-well assay plate. Add 100 μL of 4×10⁵ cells/mL in culture media to the plate, seeding 4×10⁴ cells/well. Incubate at 37° C. with 5% CO₂ overnight. On the day of the assay, prepare 2× of a 5-fold serial dilution of the antibodies in assay buffer for a total of 11 data points at each of pH 6.0 and pH 7.4. The initial antibody concentration for HCT116 and CHO hEpCAM cells was 250 nM. For CHO cynoEpCAM cells, the starting concentration was 1250 nM. Cover with sterile plate sealer and plate on ice.
Thaw two vials of frozen TCR/CD3 effector cells in a 37° C. water bath for 3 minutes, gently mix the cell suspension and transfer the content to two 1.7 mL sterile Eppendorf tubes, spin down with a benchtop mini centrifuge at 6000 rpm for 30 seconds to pellet the cells. Aspirate the supernatant and resuspend one vial with 4 mL assay media at pH 6.0 and the other vial with 4 mL of assay media at pH 7.4.
Remove the culture media from the assay plate carefully without disturbing the cells.
Add 25 μL/well of 2× diluted antibodies according to the layout.
Add 25 μL/well of the TCR/CD3 effector cell suspension according to the layout.
Cover the plate and incubate at 37° C. with 5% CO₂ for 6 hours.
Pre-warm the Bio-Glo™ buffer and substrate from Promega at room temperature.
Add the Bio-Glo™ buffer to the substrate when the 6 hours incubation ends and mix well until the substrate is completely dissolved.
Add 50 μL/well Bio-Glo™ substrate to the assay plate according to the layout, incubate for 5 minutes at room temperature protected from light on a shaker at 100 rpm.
Record the luminescence signal using Molecular Devices SpectraMax i3X reader.

Data Analysis The functional activities of the test articles at pH 6.0 and pH 7.4 were determined by plotting bioluminescence units (RLUs) against the antibody concentrations. The EC50 values were calculated using the nonlinear fit (variable slope, four parameters) model built into Graph Pad Prism software version 9.2.0.

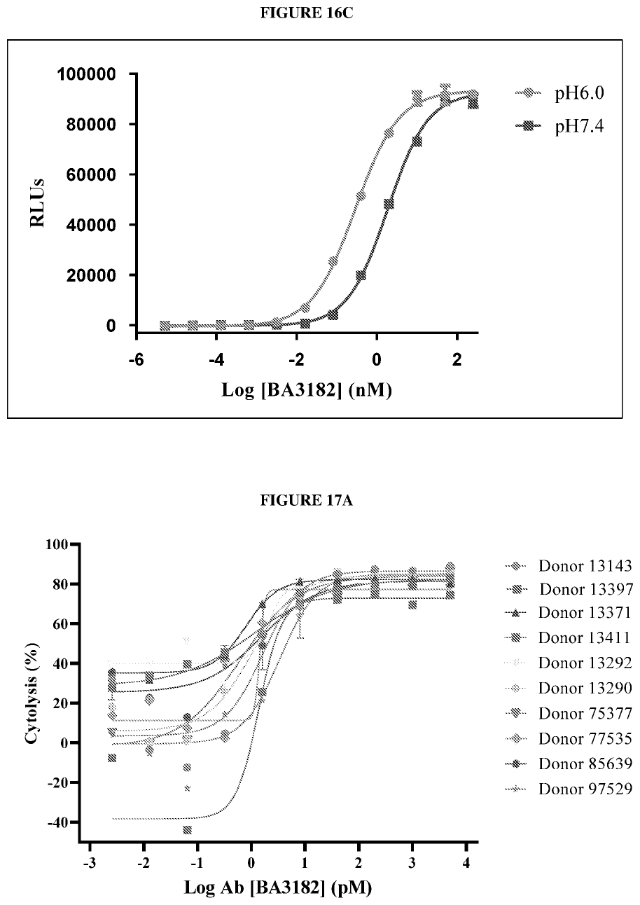

Results Three independent experiments were performed for each cell type. The functional activity of BA3182 was determined using a Promega T cell Activation Bioassay. The results showed that BA3182 had higher activity in an acidic pH which mimics the tumor microenvironment (pH 6.0). On the contrary, BA3182 was less active at a normal physiological pH (neutral pH, pH 7.4) (FIGS. 16A, 16B, 16C). The mean EC50 of BA3182 for CHO hEpCAM cells was 0.049 nM at pH 6.0 and 0.383 nM at pH 7.4 (Table 8). For CHO cynoEpCAM cells, the mean EC50 was 0.576 nM and 9.647 nM, at pH 6.0 and pH 7.4, respectively (Table 9). The mean EC50 of BA3182 for HCT116 cells was 0.273 nM at pH 6.0 and 1.988 nM at pH 7.4 (Table 10). No effect of the isotype control bispecific antibody was observed. Finally, no activity of BA3182 was detected against naive CHO cells (data not shown).

TABLE 8
Potency of BA3182 in mediating T cell Activation
in CHO-hEpCAM cells at pH 6.0 and pH 7.4.
BA3182 EC50 (nM) in CHO hEpCAM cells

| | pH 6.0 | pH 7.4 |
|---|---|---|
| Experiment-1 | 0.051 | 0.358 |
| Experiment-2 | 0.049 | 0.387 |
| Experiment-3 | 0.048 | 0.403 |
| Mean ± SD | 0.049 ± 0 0.002 | 0.383 ± 0.023 |

TABLE 9
Potency of BA3182 in mediating T cell Activation
in CHO-cynoEpCAM cells at pH 6.0 and pH 7.4.
BA3182 EC50 (nM) in CHO cynoEpCAM cells

| | pH 6.0 | pH 7.4 |
|---|---|---|
| Experiment-1 | 0.545 | 9.457 |
| Experiment-2 | 0.597 | 8.825 |
| Experiment-3 | 0.586 | 10.660 |
| Mean ± SD | 0.576 ± 0.027 | 9.65 ± 0.932 |

TABLE 10
Potency of BA3182 in mediating T cell Activation
in HCT116 cells at pH 6.0 and pH 7.4.
BA3182 EC50 (nM) in HCT116 cells

| | pH 6.0 | pH 7.4 |
|---|---|---|
| Experiment-1 | 0.292 | 1.857 |
| Experiment-2 | 0.288 | 2.146 |
| Experiment-3 | 0.238 | 1.962 |
| Mean ± SD | 0.273 ± 0.030 | 1.99 ± 0.147 |

### Example 22. Cytotoxicity Assay Using Human and Cynomolgus PBMCs

This example shows the functional activity of BA3182 bispecific antibody using an in vitro cytotoxicity assay in which human or cyno peripheral blood mononuclear cells (PBMCs) are activated upon in vitro stimulation with BA3182 antibody to kill EpCAM expressing cells. To this end, HCT116 cells or CHO cells expressing cynoEpCAM were treated with serially diluted BA3182 antibody, followed by the addition of human or cyno PBMCs. The cocultures were incubated in culture media at pH 6.5 mimicking the pH of the tumor microenvironment, or at pH 7.4 mimicking a normal physiological pH. BA3182 mediated cytolysis was monitored in real time using Agilent xCELLigence Real Time Cell Analysis technology (RTCA). The rate of target cell cytolysis was calculated by referencing the target cell growth without treatment at a defined time point. The percentage of cytolysis obtained was plotted against the concentrations of BA3182. Data was fitted using a four parameters nonlinear regression model with variable slope, to allow the determination of EC50 and EC20 values. The mean EC50 of BA3182 mediated cytolysis of HCT116 was 1.41 μM at pH 6.5 (using 10 different human PBMCs donors) and 5.61 μM at pH 7.4 (using 6 different human PBMCs donors). The mean EC20 of BA3182 mediated cytolysis of HCT116 was 0.54 μM at pH 6.5 (using 10 different human PBMCs donors) and 4.09 μM at pH 7.4 (using 6 different human PBMCs donors). The mean EC50 of BA3182 mediated cytolysis by cyno PBMCs of CHO cells expressing cynoEpCAM was 17.39 μM at pH 6.5 and 184.73 μM at pH 7.4.

### Materials

BA3182 Isotype control bispecific antibody was produced by Evitria (Zurich, Switzerland), lot number #10229, 3 mg/mL.
RPMI-1640: ThermoFisher Gibco Cat #11875-085, Lot #2463433
DMEM: ThermoFisher Gibco Cat #11965-084, Lot #2346179
FBS: Sigma, Cat #12306C-500 mL, Lot #16J367
100×NEAA: Gibco, Cat #11140-050 Lot #2337217
100 mM Sodium pyruvate: Gibco, Cat #11360-070, Lot #2323639
Cell culture grade water: Corning Cat #25-055-CM, lot #17216006
12N HCl: VWR, Cat #87003-251, Lot #4118020
50 mL conical tube: BioPioneer Cat #CNT-50, Lot #10272021
E-Plate View 96: Agilent, Cat #300601010, Lot #20211132
Syringe filter: Celltreat Cat #229747, Lot #211004-052-1A
pH Meter: ORION STAR A111, ThermoFisher, serial #J17162
Trypan blue stain: Gibco Cat #15250-061, lot #1861515
Adhesive plate sealing films for microplate: E&K Scientific, Seal Plate, Cat #T396100 Lot #SG262G
Sample dilution block: Corning, Cat #3956, Lot #07718000
Agilent xCELLigence Real-Time Cell Analysis (RTCA) MP Analyzer
Plate reader: SpectraMax i3X, Molecular Devices
PBMC pH 6.5 assay media: RPMI-1640+10% FBS, adjust to pH 6.5 using 6N HCl, sterilize with 0.2 μm Syringe filter.
PBMC pH 7.4 assay media: RPMI-1640+10% FBS, adjust to pH 7.4 using 6N HCl, sterilize with 0.2 μm Syringe filter.
CHO cynoEpCAM culture media: DMEM+1×NEAA (0.1 mM non-essential amino acid solution)+1× sodium pyruvate (1 mM)+10% FBS+1 mg/mL G418
HCT116 culture media: McCoy's 5A+10% FBS
CHO cynoEpCAM clone #3, BioAtla, passage #13
HCT116, human colon carcinoma, ATCC, Cat #CCL-247™, passage #11
Human PBMC: Precision for Medicine, Cat #93000-10M, lot #13143; 2010113397; 2010113371; 2010113411; 2010113292; 2010113290; 201675377; 201877535; 201885639; 201897529
CynoPBMC: Worldwide Primates Inc, Cat #CA-10, lot #207117-1

### Methods

### Cell Culturing and Plating.

One day before the assay, detach the target cells and resuspend in culture media as a single cell solution.
Count cells with a hemocytometer using Trypan blue staining.
Adjust the target cell concentration to 4×10⁴ cells/mL and add 100 μL into the 96-well assay plate, seeding 4×10³ cells/well in cell culture media.
Incubate the target cells at 37° C. with 5% CO₂ overnight.

### PBMC Resting

Thaw the PBMCs in a water bath at 37° C. for 3 minutes and add to 10 mL warm PBMC assay media (pH 7.4) in a dropwise manner, swirl the conical tube constantly to help the cells adapt to the culture media temperature.
Pipette up and down using a 10 mL pipette until there are no visible clumps.
Collect the cells using a centrifuge at 1400 rpm for 10 minutes, discard the media supernatant.
Resuspend the cell pellet with 10 mL fresh warm media, and pipette to separate the cells. Incubate at 37° C., 5% CO₂ incubator overnight (between 18-20 hrs.).
Before plating, count the cells and determine cell viability.
Collect the cells using a centrifuge at 1400 rpm for 10 minutes, discard the media supernatant.
Resuspend cells with designated volume of pH media to a density of 4×10⁵ cells/mL.

### Test Antibody Preparation and Assay Setup

Serially dilute test articles to 2× the starting concentration in pH 6.5 or pH 7.4 assay media creating a stock solution starting at 10 nM for HCT116/human PBMC cells and 200 nM for CHO cyno EpCAM/cyno PBMC cells, respectively. Perform 5-fold dilution for a total of 11 data points. Dispense 100 μL/well serially diluted test articles and add 100 μL/well of the PBMC suspension. Final volume is 200 μL/well.
For total lysis cocultures of PBMC and target cells were treated with 1% SDS. Incubate at 37° C. with 5% CO₂ for 100 hrs. with data collection every 15 minutes for a total of 400 reads.

Results The functional activity of BA3182 was determined using CD3 mediated PBMC killing of target cells. Human PBMCs from six heathy subjects were cocultured with HCT116 and various concentrations of BA3182 for 120 h. Cell growth was determined using the XCELLigence Real-Time Cell Analysis technology. Cytotoxicity was determined using the following function:

$$( \frac{cell index of untreated cultures - cell index of BA 3182 treated cultures}{cell index of untreated cultures - cell index of 100 \% lysis} ) \times 100$$

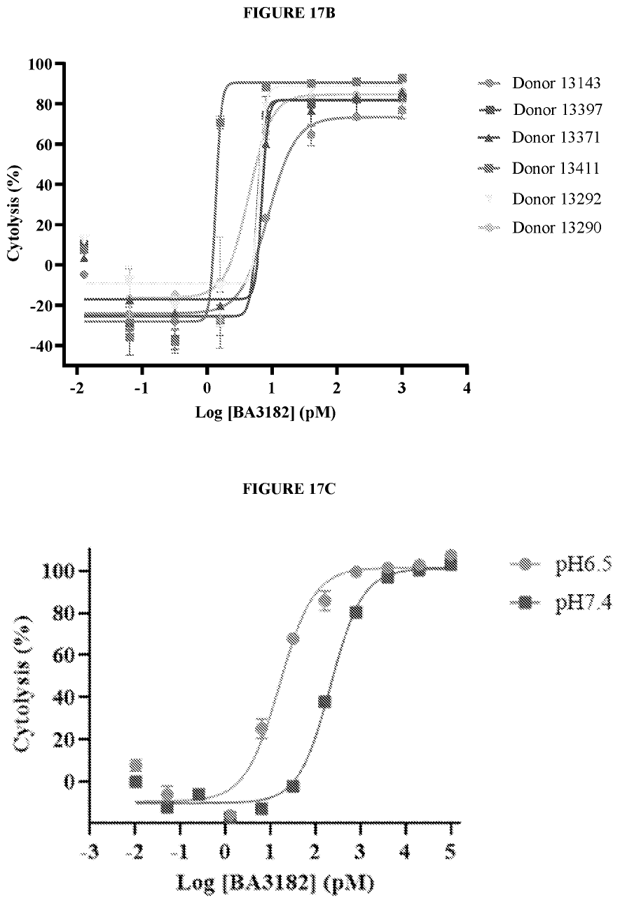

The results showed that BA3182 had higher activity at a tumor environment pH (acidic pH, pH 6.5). Alternatively, BA3182 was less active in normal physiological pH (neutral pH, pH 7.4) (FIGS. 17A-17C). The mean EC50 of BA3182 in tumor microenvironment (pH 6.5) for HCT116 cells was 1.41 pM and 5.61 pM at pH 7.4, the normal physiological pH. The mean EC20 of BA3182 in tumor microenvironment (pH 6.5) for HCT116 cells was 0.54 pM and 4.09 pM at pH 7.4 (Table 11).

TABLE 11
BA3182 potency in inducing cytolysis of EpCAM expressing cells.
A.
BA3182 EC₅₀ and EC₂₀ (pM) in HCT116 cells

| | pH 6.5 | | pH 7.4 | |
|---|---|---|---|---|
| PBMC | EC₅₀ | EC₂₀ | EC₅₀ | EC₂₀ |
| Donor ID | (pM) | (pM) | (pM) | (pM) |
| 13143 | 1.66 | 0.53 | 8.73 | 4.45 |
| 13397 | 1.19 | 0.13 | 5.9 | 5.24 |
| 13371 | 0.79 | 0.31 | 6.96 | 5.82 |
| 13411 | 1.39 | 0.7 | 1.36 | 1.21 |
| 13292 | 2.05 | 0.7 | 6.35 | 5.34 |
| 13290 | 1.03 | 0.27 | 4.37 | 2.48 |
| 75377 | 0.6 | 0.11 | N/A | N/A |
| 77535 | 1.52 | 1.27 | N/A | N/A |
| 85639 | 0.88 | 0.19 | N/A | N/A |
| 97529 | 2.95 | 1.18 | N/A | N/A |
| Mean ± SD | 1.41 ± 0.69 | 0.54 ± 0.42 | 5.61 ± 2.52 | 4.09 ± 1.84 |

B.
BA3182 EC50 (pM) in CHO cynoEpCAM cells

| | pH 6.5 | pH 7.4 |
|---|---|---|
| Experiment-1 | 18.86 | 172.40 |
| Experiment-2 | 15.94 | 214.70 |
| Experiment-3 | 17.37 | 167.10 |
| Mean ± SD | 17.39 ± 1.46 | 184.73 ± 26.09 |

### Example 23. PBMC Cytokine Release Assay

In this example the release of IL-2, IL-6, IL-10, INFγ, and TNFα cytokines upon stimulation of human peripheral mononuclear cells (PBMCs) with various concentrations of BA3182 in the presence of EpCAM expressing cells was investigated. To this end, HCT116 cancer cells were seeded in tissue culture plates and incubated overnight at 37° C. with 5% CO₂. The next day, growth medium was removed and human PBMCs from 9 donors were added to establish co-cultures in culture medium at pH 6.5, representative of tumor microenvironment pH. BA3182 or isotype control antibodies were serially diluted, added to the plates and the cultures were maintained at 37° C. with 5% CO₂. After 48 h the plates were centrifuged to pellet the cells, supernatants were collected, transferred to a fresh plate, and stored at −80° C. until analysis of cytokine levels. Cytokine concentrations were determined with R&D Systems Quantikine ELISAs Kit and BA3182 EC50 values were calculated using the GraphPad prism software. BA3182 was able to induce IL-2 and INFγ cytokines release in nine donors at pH 6.5. Baseline IL-6, IL-10 and TNFα cytokines were detected, and the levels varied between donors. No effect of BA3182 or isotype in inducing these cytokines was observed.

### Materials

TABLE 12
Antibodies

| | | Conc. | |
|---|---|---|---|
| Protein | Lot. No. | (mg/ml) | Buffer |
| BA3182 | 70B-010- | 1.03 | 20 mM Histidine, 5% |
| | 002-T01-01 | | Trehalose, 0.05% PS80; pH 6.0 |
| Isotype* | 21-0095 | 1.64 | PBS 100 mM Arginine |
| BA-HEL- | | | |
| BF130 | | | |

*Isotype: Non-CAB human IgG anti-Hen Egg white lysozyme (HEL) containing anti-hCD3 scFv.

TABLE 13
Reagents and Consumables

| Name | Vendor | Cat. No. | Lot. No. |
|---|---|---|---|
| Human INFγ Quanitkine | R&D | SIF50C | P310840 |
| ELISA | Systems | | |
| Human IL-2 Quantikine | R&D | S2050 | P308937 |
| ELISA | Systems | | |
| Human IL-10 Quantikine | R&D | S1000B | P331853 |
| ELISA | Systems | | |
| Human TNFα Quantikine | R&D | STA00D | P325919 |
| ELISA | Systems | | |
| Human IL-6 Quantikine | R&D | S6050 | P327315 |
| ELISA | Systems | | |
| McCoy's 5A Medium | Gibco | 16600-082 | 2323631 |
| RPMI 1640 Medium | Gibco | 11875-085 | 2403433 |
| FBS | Sigma | 12306C | 16C363 |
| PBS, pH 7.4 | Gibco | 10010-023 | 2418661 |
| Dynabeads Human T-Activator | Gibco | 11131D | 01029596 |
| CD3/CD28 | | | |
| 96-well Costar Flat Bottom | Corning | 3596 | — |
| Plates | | | |
| Trypan Blue Stain | Gibco | 15250 | 2337341 |
| 0.25% Trypsin -EDTA | Gibco | 25200-056 | 2323575 |
| PE-anti Human EpCAM Clone | Biolegend | 324206 | B342477 |
| 9C4 | | | |
| PE-mouse IgG2bκ Isotype | Biolegend | 400314 | B324362 |
| Clone MPC-11 | | | |
| BSA | Sigma | A9647 | SLBV4996 |
| HCl | VWR | 87003-251 | 4118020 |

TABLE 14
Equipment

| Name | Vendor | Model No. | S/N |
|---|---|---|---|
| Microplate | Molecular Devices | SpectraMax i3x | 363703462 |
| Reader | | | |
| Flow cytometer | ACEA | NovoCyto 20602 | 605 6 |

### Methods

Cell Culturing The HCT116 cells were maintained in McCoy's 5A culture medium supplemented with 10% FBS. The cells were routinely sub-cultured twice per week. The cells were harvested during the exponential growth phase and counted for plating and for determination of surface expression of EpCAM.

Cell Staining Prior to plating the target cells for the assay, the surface expression of EpCAM was confirmed by flow cytometry. To this end, the culture medium was removed from T75 HCT116 culture flasks, and the cell layer was briefly rinsed with PBS pH 7.4. One mL of 0.25% trypsin-EDTA was added to each flask and the cells were returned to 37° C. until the cell layer was dispersed. Trypsin reaction was blocked with 9 mL of culture medium and the cells were resuspended by gentle pipetting. For FACS staining, 2×10⁵ HCT116 cells were added to 1.5 mL Eppendorf tubes and washed with PBS pH 7.4. Cells were spun, supernatant removed, and the cells were resuspended with 100 μL of PBS+1% BSA containing 2 μL of PE-anti Human EpCAM Clone 9C4 or PE-mouse IgG2bk Isotype Clone MPC-11. Cells were incubated for one hour on ice, in the dark, with shaking at 100 rpm. After incubation, the cells were washed three times with 1 mL PBS pH 7.4. Then, the cells were resuspended in 120 μL PBS+1% BSA and analyzed on a NovoCyte flow cytometer.

Incubation with Antibodies HCT116 cells were seeded at a density of 6000 cells/well in 96-well tissue culture plates in McCoy's 5A culture medium supplemented with 10% FBS and incubated overnight at 37° C. with 5% CO₂. After overnight culture, the growth medium was removed and 30,000 cells/well of human PBMCs were added in RPMI 1640+10% FBS medium at pH 6.5 (reflective of tumor microenvironment). PBMC cells were added at a 5:1 ratio of PBMC to target cell. BA3182 or isotype control antibodies were serially diluted from 150 to 0.008 nM (3-fold dilutions) in RPMI 1640+10% FBS at pH 6.5, added to the plates and the cultures maintained at 37° C. with 5% CO₂. After 48 h incubation with the test antibodies, the plates were centrifuged at 2000 rpm for 3 minutes at ambient temperature to pellet the cells, and the supernatants were removed to fresh 96-well plates and stored at −80° C. until analysis of cytokine levels.

Measurement of Cytokines Cytokine concentrations were determined using Quantikine™ ELISA assays following the manufacturer's protocol. Supernatants were diluted in assay diluent as required to obtain values in the linear range of the standard curve of the assay. EC50 values were calculated from non-linear 4-parameter regression curves of cytokine standards using Graph Pad Prism software version 9.0.

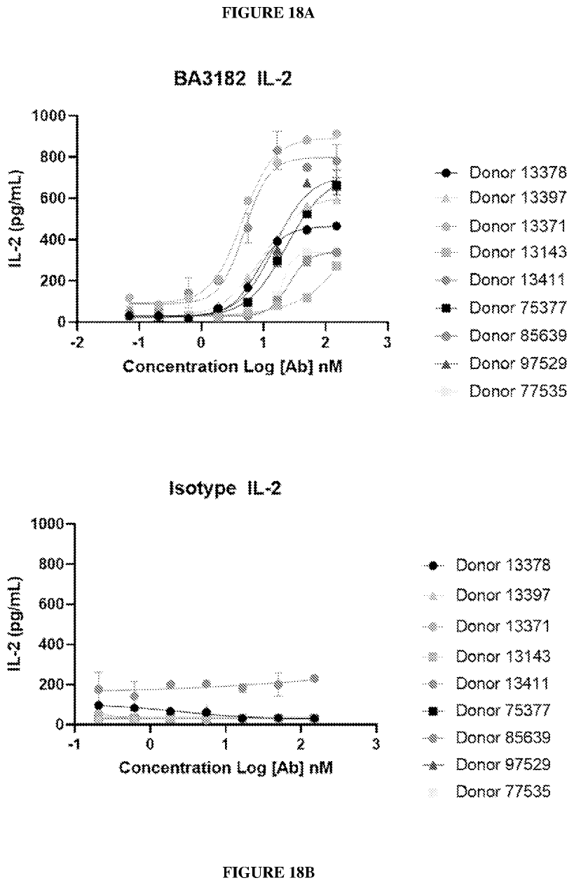

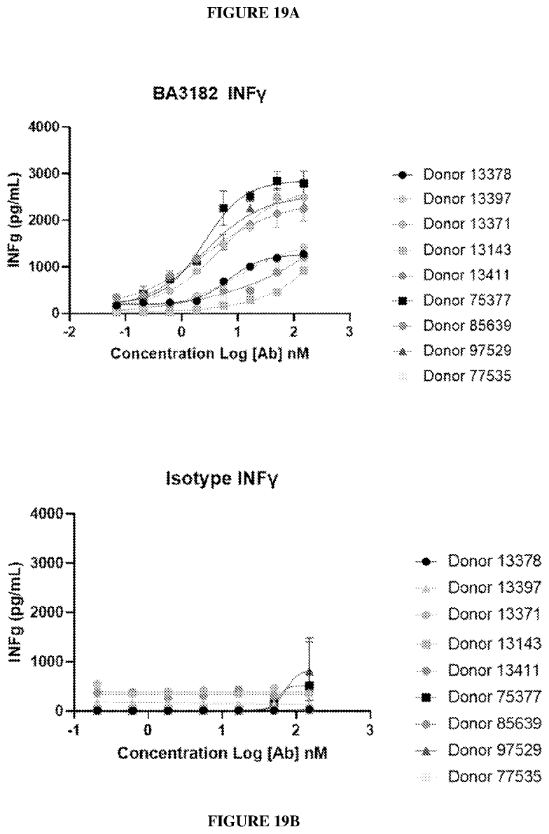

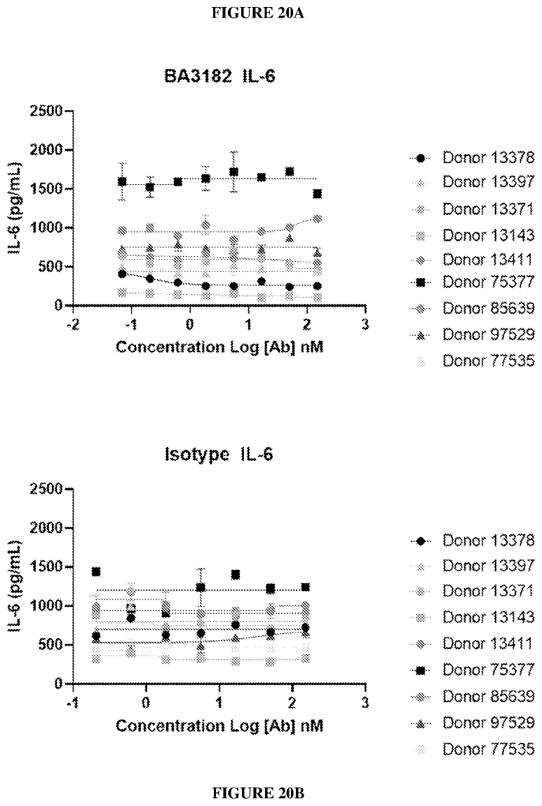

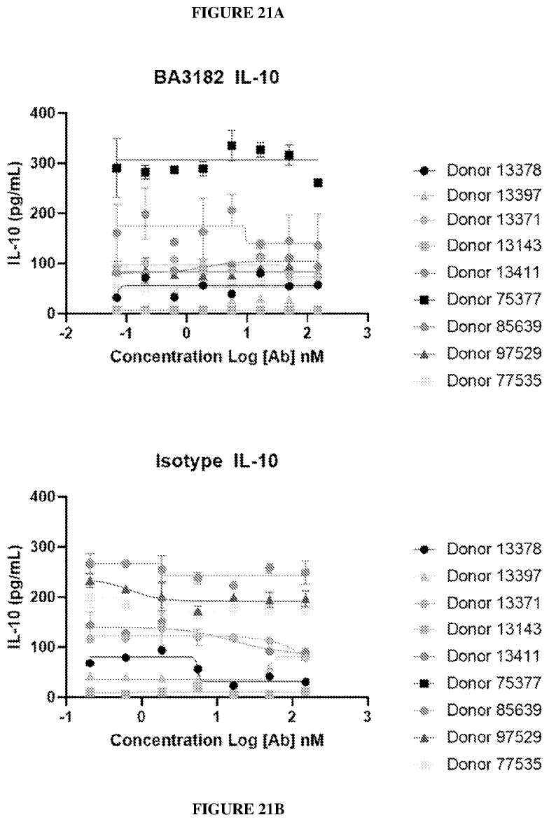

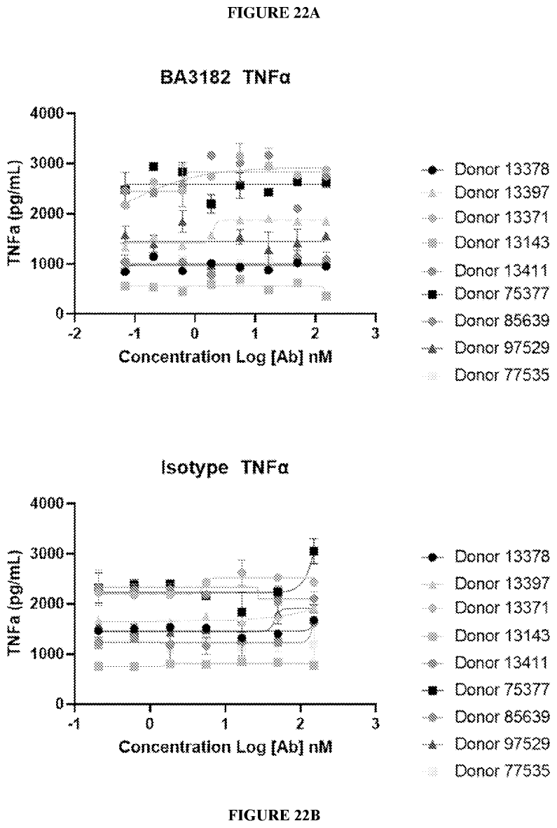

Results BA3182 was able to induce release of IL-2 and INFg cytokines by human PBMCs cocultured with human colon cancer cell line HCT116 at pH 6.5 (tumor microenvironment pH). No cytokine response was induced by stimulation of human PBMCs with BA3182 in the absence of HCT116 cells (data not shown). Isotype control antibody, which is a bispecific antibody anti-Hen egg white lysozyme/CD3, did not induce IL-2 or INFg release (FIGS. 18A-18B and FIGS. 19A-19B). Neither BA3182 nor isotype antibodies had any measurable effect on the release of IL-6, IL-10 or TNFα beyond background levels (FIGS. 20A-20B, 21A-21B and 22A-22B). Low nanomolar EC50 values of BA3182 were determined for the induction of IL-2 and IFNg by PBMCS from 9 human donors tested at pH 6.5. PBMCs from a 10th donor did not respond to any stimulus including the positive control of CD3/CD28 immobilized microbeads (data not shown) when cultured with HCT116 cancer cells and this donor was eliminated from the study. The EC50 values obtained at pH 6.5 for the induction of IL-2 and INFg by BA3182 varied with the donor and are presented in Table 15. This data demonstrates that in vitro, BA3182 antibody is a potent inducer of IL-2 and IFNγ production by human PBMCs in the presence of EpCAM antigen. BA3182 does not induce pro-inflammatory cytokine production such as IL-6 and TFN-alpha or IL-10 anti-inflammatory cytokine by human PBMCs.

TABLE 15
BA3182 EC50 values for the induction IL-2 and INFg
cytokine production by human PBMCs co-cultured with
HCT116 cancer cells in the presence of BA3182.

| Donor | EC50 IL-2 (nM) | EC50 INFγ (nM) |
|---|---|---|
| 3378 | 7.85 | 6.45 |
| 3397 | 9.82 | 10.05 |
| 3371 | 4.56 | 5.28 |
| 3143 | ND | ND |
| 3411 | 4.92 | 2.54 |
| 75377 | 23.20 | 2.53 |
| 85639 | 25.56 | ND |
| 97529 | 14.99 | 2.52 |
| 77535 | 19.66 | 24.03 |

ND = not determined from curve fit.

### Example 24. C1q Affinity ELISA

In this Example, binding of human complement protein C1q to BA3182 was determined using a C1q affinity ELISA assay. ELISA assay plates were coated with BA3182 or a positive control antibody (B12). Purified human C1q protein was serially diluted and added to the plates. Binding of C1q protein to the antibodies was detected by the addition of sheep anti-human C1q-HRP conjugated antibody. Colored product was generated by the addition of TMB colorimetric substrate. The OD absorbance in each well was determined using a microplate reader. Data was analyzed and non-linear 4 parameter regression curves were generated using Graph Pad Prism software. Results indicate that C1q bound poorly to BA3182. This result was expected as the glycosylation site at position 297 was mutated from asparagine (N) to glutamine (Q). This mutation has been proven successful in abrogating effector functions such as Complement Dependent Cytotoxicity (CDC).

### Materials

### Antibodies

| | Conc. | |
|---|---|---|
| Protein | (mg/ml) | Buffer |
| BA3182 | 1.03 | 20 mM Histidine, 5% |
| (EpCAM × CD3 Bispecific | | Trehalose, 0.05% PS80; pH |
| Ab) | | 6.0 |
| B12 (hIgG_{1, k} Anti-gp120) | 5.9 | PBS 100 mM Arginine |

### Reagents and Consumables

| Name | Vendor | Cat. No. | Lot. No. |
|---|---|---|---|
| Human C1q | Complement | A100 | 57 |
| | Technology | | |
| ELISA Plate | Thermo | 269787 | 1223409 |
| | Scientific | | |
| Blocker Casein in | Thermo | 37528 | WF329342 |
| PBS | Scientific | | |
| Sheep anti human | Creative | CPBT- | LLE12158918 |
| C1q Ab, HRP | Diagnostics | 65026SH | |
| Carbonate- | Sigma | C3041- | SLB23401 |
| Bicarbonate | | 50CAP | |
| buffer | | | |
| HCl | VWR | 87003-251 | 4118020 |
| PBS pH 7.4 | Gibco | 10010-023 | 2418661 |
| Tween 20 | EMD | 655204-100mL | 3739705 |

Methods ELISA plates were coated with 100 μL/well of BA3182 or B12 antibodies at 3 μg/mL in carbonate-bicarbonate buffer and incubated overnight at 4° C. The next day, plates were blocked with 300 μL/well of Blocker Casein buffer and incubated at room temperature for 1 h. After this period, plates were washed three times with 300 μL/well of PBS with 0.05% Tween 20. C1q was half-log titrated in Blocker Casein buffer (600, 189.75, 60.01, 18.98, 6.00, 1.90, 0.60, 0.19, 0.06 and 0.02 g/mL); 100 μL/well of each dilution was added to the plates and incubated at room temperature for 2 h. The plates were then washed and subsequently incubated with 100 μL/well of secondary antibody sheep anti-human C1q Ab-HRP diluted 1:200 at room temperature for 1 h. After washing, 100 μL/well of 3,3′,5,5′ tetramethylbenzidine (TMB) substrate was added. Plates were incubated in the dark at room temperature for 5-20 min, and the enzymatic reaction was stopped with 50 μL of 2M HCl. The absorbance at 450 nm was read using a SpectraMax i3x, Molecular Device microplate reader. Data was analyzed and non-linear 4 parameter regression curves were generated using Graph Pad Prism software version 9.0.

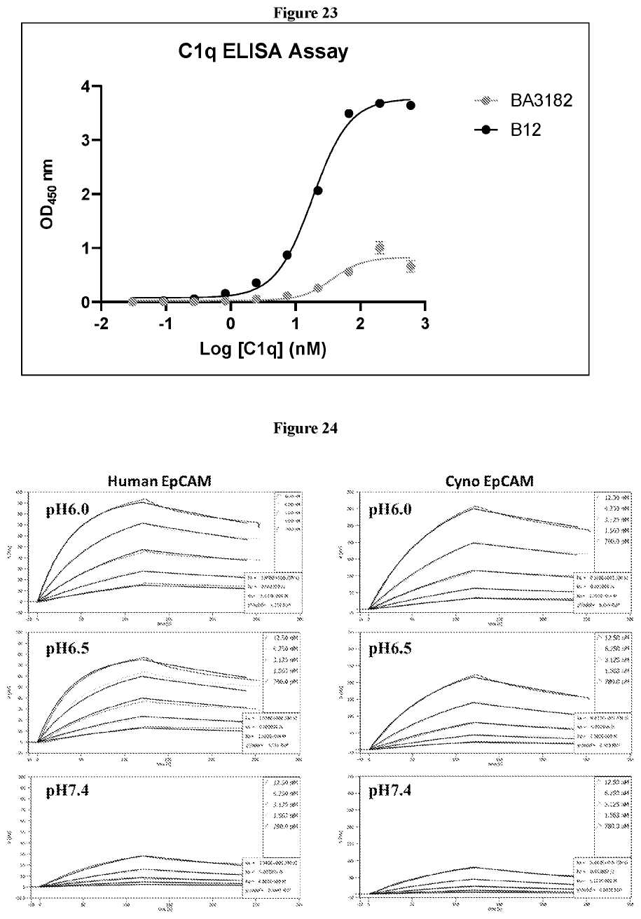

Results Molecular weight of 410 kDa was used to calculate the molar concentration of C1q, and 450 nm absorbance values were used to generate four-parameter regression curves for the calculations of EC50 values. C1q protein bound strongly to the positive control antibody B12, generating a good dose-response curve (FIG. 23). B12 is a human IgG1, k anti-gp120 glycoprotein, and binding of C1q to this antibody was expected. C1q bound poorly to the BA3182 (FIG. 23), suggesting that the affinity of C1q to BA3182 antibody is low. The calculated EC50 value for the binding affinity of C1q to BA3182 was 12.89 times higher than the EC50 for the binding affinity of C1q to B12, 613.1 nM versus 47.55 nM, respectively (Table 16). BA3182 antibody is a human IgG_{1.k} aglycosylated mutant containing the N297Q mutation which has been shown to reduce the binding affinity of human IgG₁ to C1q protein, inhibiting complement fixation and activation.

TABLE 16
Binding Affinity of C1q to BA3182

| Antibody | EC50 (nM) |
|---|---|
| BA3182 | 613.1 |
| B12 | 47.55* |

### Example 25. Analysis of Binding Kinetics by SPR

The objective of this example was to determine the binding kinetics of BA3182 to human and cynomolgus EpCAM and CD38/6 heterodimer at pH 6.0, pH 6.5, and pH 7.4 using surface plasmon resonance (SPR). The ligands were immobilized on the surface of a flat amine sensor chip followed by the injection of titrated concentrations of BA3182. The binding interaction between BA3182, the ligands, and a control surface were monitored in real time. The binding kinetics (on-rate kd, off-rate ka, affinity K_{D}) were calculated using a 1:1 Langmuir model built into the analysis software.

BA3182 binds to human EpCAM under tumor microenvironment conditions (acidic pH) with ~1.2 nM affinity. At normal physiological pH, the affinity drops 5.5-fold to 6.7 nM. Binding to cynomolgus EpCAM shows a similar trend: binding affinity at acidic pH is ~2-3 nM and drops 6-fold to ~12 nM at normal physiological pH. BA3812 binds to human CD3 under tumor microenvironment conditions (acidic pH) with 8-9 nM affinity. At normal physiological pH the affinity is ~35 nM. Binding to cynomolgus CD3 shows a similar trend: binding affinity at acidic pH is ~11 nM and drops 3-fold to ~30 nM at normal physiological pH.

### Materials

### Antibodies

BA3182 was used at a stock concentration of 1.03 mg/mL.

### Instruments and Reagents

| Name | Vendor | Cat. No. | Lot No. |
|---|---|---|---|
| SPR32 Pro | Bruker | NA | NA |
| Instrument | | | |
| Flat Amine Chip | Bruker | SPR-AS-AMF | 10BB10215A |
| Human EpCAM-His | BioAtla | n/a | 210158 |
| Human CD3-hFc | BioVision | P11830-500 | Y20204090 |
| Cyno EpCAM-His | BioAtla | n/a | 8272019 |
| Acetate 5.0 | Bruker | 1862646 | 10DA11285N |
| Acetate 5.5 | Bruker | 1862646 | 10DA11285N |
| Ethanolamine-HCl | Bruker | 1862634 | 10DA1133C |
| EDC | Bruker | 1862634 | 10DA11292A |
| NHS | Bruker | 1862634 | 10DA11292A |
| PBS | Thermofisher | 70011-044 | 2323767 |
| Twecn-20 | Sigma | P9416-50ML | SLCJ0231 |
| Glycine | Sigma | G7126-100G | SLCC0460 |
| Sodium Bicarbonate | Thermofisher | 25080-094 | 2390131 |
| Sodium chloride | VWR | 0241-500G | 22A3156549 |
| Hydrochloric acid | VWR | 470301-264 | 4107B75 |
| PES bottle filter | Cell Treat | 229708 | 211104-208-A |
| Sonicator vacuum | Branson | N/A | N/A |
| Amicon Ultra-15, 30K | Millipore | UFC903024 | RIPB80727 |
| | Sigma | | |

Buffers. SPR Running buffers containing sodium bicarbonate at pH 6.0, pH 6.5, and pH 7.4 were prepared as described below immediately before use as the pH of the buffer changes during storage.

### PBST-SB-75 pH 6.0

1. Add 16.6 mL 7.5% Sodium Bicarb solution to 483.4 mL 1×PBS.
2. Add 250 μLL Tween-20.
3. Add 2.19 g NaCl.
4. Adjust pH to 5.9 with 6N HCL.
5. Filter with 0.22 μM PES bottle filter.
6. Degas using a sonicator and vacuum for 5 minutes.
7. Remove a 50 mL aliquot for sample preparation.

### PBST-SB-75 pH 6.5

1. Add 16.6 mL 7.5% Sodium Bicarb solution to 483.4 mL 1×PBS.
2. Add 250 μL Tween-20.
3. Add 2.19 g NaCl.
4. Adjust pH to 6.4 using 6N HCL.
5. Filter with 0.22 μM PES bottle filter.
6. Degas using a sonicator and vacuum for 5 minutes.
7. Remove a 50 mL aliquot for sample preparation.

### PBST-SB-75 pH 7.4

1. Add 16.6 mL 7.5% Sodium Bicarb solution to 483.4 mL PBS
2. Add 250 μL Tween-20.
3. Add 2.19 g NaCl.
4. Adjust pH to 7.35 using 6N HCL.
5. Filter with 0.22 μM PES bottle filter.
6. Degas using a sonicator and vacuum for 5 minutes.
7. Remove a 50 mL aliquot for sample preparation.

### Sensor Regeneration Solution

### 10 mM Glycine pH 2.0

Methods. The SPR32 Pro instrument has eight flow channels (1-8) with four detection spots each (A, B, C, D). The 32 detection spots can be addressed individually or in groups. The surface of each new flat amine sensor chip was preconditioned according to the manufacturer's recommendations using the built-in method before immobilization of the ligands. PBST pH 7.4 was used as running buffer for the preconditioning and the immobilization of EpCAM or CD3.

A, B, and C detection spots were activated by injecting a mixture of EDC/NHS (250 mM/50 mM) for 240 s at a flow rate of 25 μL/min. No protein was immobilized on the control surface (detections spots 1A-8A). Human EpCAM-His was diluted to 0.5 μg/mL in 10 mM NaAc pH 5.0 and injected over detection spots 1B-8B for 240 s at a flow rate of 25 μL/min. CynoEpCAM-His was diluted to 0.5 μg/mL in 10 mM NaAc pH 5.5 and injected over detection spots 1C-8C for 240 s at a flow rate of 25 L/min. A, B, and C detection spots were blocked by injecting ethanolamine (1M) for 240 s at a flow rate of 25 μL/min. A, B, C, and D detection spots were activated by injecting a mixture of EDC/NHS (250 mM/50 mM) for 240 s at a flow rate of 25 μL/min. No protein was immobilized on the control surface (detections spots 1A-8A). Human CD3-hFc fusion was diluted to 1 μg/mL in 10 mM NaAc pH 5.5 and injected over detection spots 1D-8D for 240 s at a flow rate of 25 L/min. CynoCD3-His was diluted to 0.05 g/mL in 10 mM NaAc pH 5.5 and injected over detection spots 1B-8B for 240 s at a flow rate of 25 μL/min. A, B, C and D detection spots were blocked by injecting ethanolamine (1M) for 240 s at a flow rate of 25 μL/min.

BA3182 was buffer exchanged into 1×PBS using a Amicon Ultra-15 spin filter (150 kDa MWCO). 1 mL of BA3182 was added to the upper reservoir followed by ~14 mL of 1×PBS. The solution was centrifuged at 4000 RPM for 12 minutes. The flow through was discarded and the procedure was repeated 3 more times. Protein concentration was determined by UV280. Buffer-exchanged BA3182 was diluted in running buffer to a starting concentration of 5 μg/mL. Subsequently, a two-fold serial dilution was performed for a total of 7 dilution points (5 μg/mL to 0.078 μg/mL).

The serial dilution of BA3182 (channel 8 highest, channel 2 lowest concentration, channel 1 running buffer) was injected over detection spots A, B, and C (flow rate 25 μL/min, contact time 120 s; off-rate measurement for 120 s). The sensor surface was regenerated by injecting 10 mM glycine pH 2.0 (flow rate 25 μL/min, contact time 15 s). The analyte injection was repeated two times (total of three analyte injections). Running buffer was injected before and after the BA3182 injections as blank analyte injection.

Detection spots A without immobilized protein were used as a control surface for reference subtraction. In addition, data with buffer only as analyte (0 nM analyte) were subtracted from each run. Double subtracted data were fitted with the provided analysis software Sierra Analyzer R3 (Bruker) using a 1:1 binding model. A molecular weight of 200 kDa was used to calculate the molar concentrations of the analytes.

Kinetic binding data from a representative experiment at each tested pH condition were entered into a SPR simulation software (www.sprpages.nl\spr-simulation) to analyze the signal drop from pH 6.0 to pH 7.4. Input data for the software include calculated on/off rates and K_{D} from an actual SPR experiment, molecular weights of analyte and ligand, amount of analyte immobilized on the sensor surface, association and dissociation time, maximum analyte concentration used, number of analyte concentrations and dilution steps. The program generates a sensorgram assuming 100% of the immobilized ligand is able to bind. For random immobilization of the ligand this is usually in the 30-40% range. The actual fraction can be determined by lowering the active concentration until the maximum signal in the simulation matches the maximum signal of the actual experiment. The active concentration of the ligand (EpCAM or CD3) at pH 6.0 was set to 100%.

Results A total of three independent SPR experiments at each pH were performed. The same sensor chip was used for all pH conditions. Binding kinetics of BA3182 to recombinant human and cynomolgus EpCAM at pH 6.0, pH 6.5, and pH 7.4 are summarized in Table 1. The binding curves for representative experiments at each pH are shown in FIG. 24. BA3182 binds to human EpCAM with an affinity of 1.30 nM at pH 6.0, 1.22 nM at pH 6.5, and 6.73 nM at pH 7.4 (Table 17) and to cynomolgus EpCAM with an affinity of 2.03 nM at pH 6.0, 2.93 nM at pH 6.5, and 11.5 nM at pH 7.4 (Table 17).

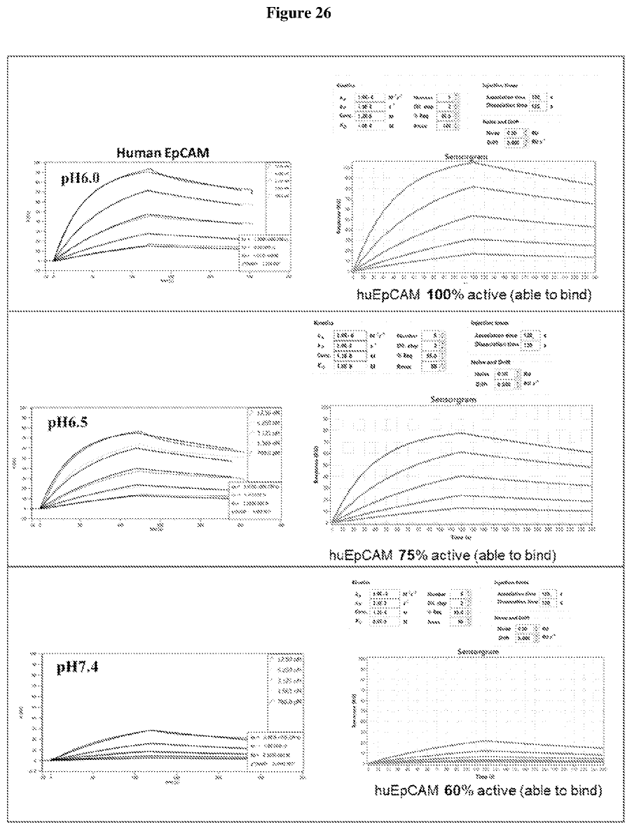

Simulations of the obtained sensorgrams show that the reduced signal at higher pH is caused by a reduction of active ligand on the chip surface (FIG. 26). This differential binding profile is expected as the antibody was engineered to have reduced binding under physiological conditions while maintaining full binding capabilities in the tumor microenvironment. BA3182 binds to human and cynomolgus EpCAM with similar affinity (within two-fold) indicating that cynomolgus monkeys are an appropriate species for toxicology testing.

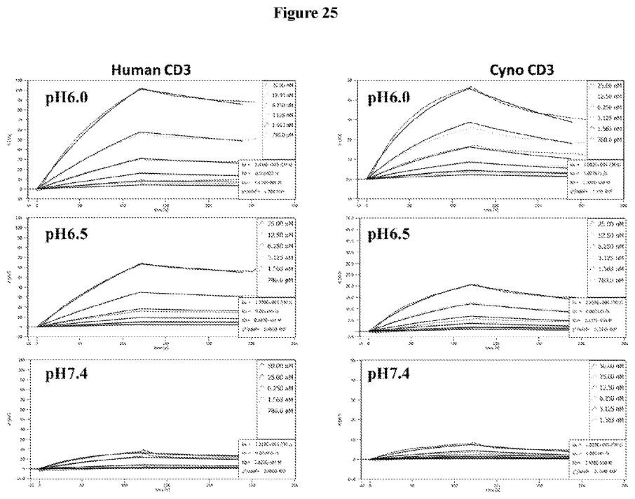

A total of three independent SPR experiments at each pH were performed. The same sensor chip was used for all pH conditions. Binding kinetics of BA3182 to recombinant human and cynomolgus CD3 at pH 6.0, pH 6.5, and pH 7.4 are summarized in Table 18. The binding curves for representative experiments at each pH are shown in FIG. 25.

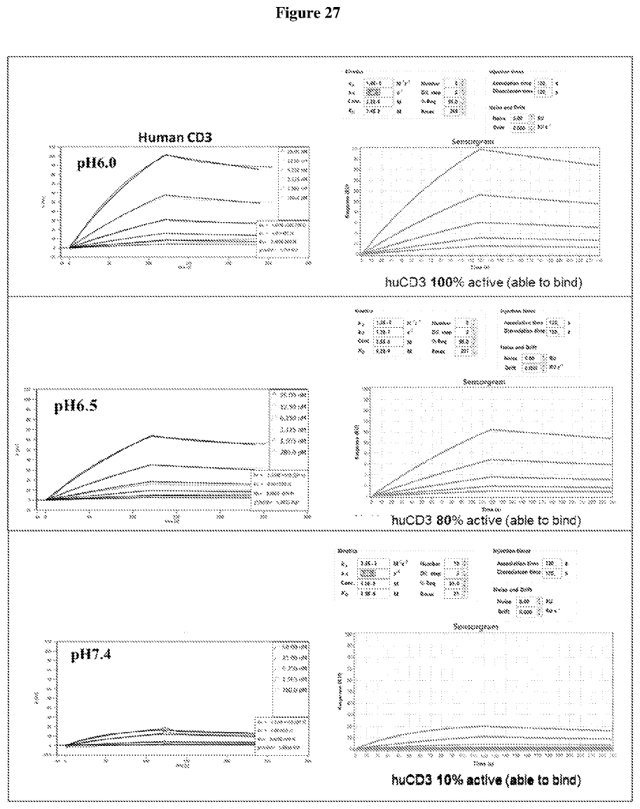

BA3182 binds to human CD3 with an affinity of 8.1 nM at pH 6.0, 9.1 nM at pH 6.5, and 35 nM at pH 7.4 (Table 18) and to cynomolgus CD3 with an affinity of 11.0 nM at pH 6.0, 11.8 nM at pH 6.5, and 28.6 nM at pH 7.4 (Table 18). At pH 7.4 the signal for most analyte concentrations was within the noise level and could not be used for K_{D} calculations. The binding data for pH 7.4 shown in Table 18 are based on only the highest concentration used. K_{D} values based on a single concentration are within two-fold of the values calculated with the whole dilution series. In addition to the lower binding affinity at higher pH, the maximum SPR signal drops from pH 6.0 to pH 7.4. (FIG. 25). Simulations of the obtained sensorgrams show that the reduced signal at higher pH is caused by a reduction of active ligand on the chip surface (FIG. 27).

TABLE 17
Affinity of BA3182 to human and cynomolgus EpCAM at different pH conditions.
The results from 3 independent experiments and the average values are listed.

| | pH 6.0 | | | pH 6.5 | | | pH 7.4 | | |
|---|---|---|---|---|---|---|---|---|---|
| | kₐ [M · s] | k_{d}[s⁻¹] | K_{D}[M] | kₐ [M · s] | k_{d}[s⁻¹] | K_{D}[M] | kₐ [M · s] | k_{d}[s⁻¹] | K_{D}[M] |
| hEpCAM exp1 | 1.91E+06 | 1.96E−03 | 1.03E−09 | 1.98E+06 | 2.04E−03 | 1.03E−09 | 4.89E+05 | 3.58E−03 | 7.32E−09 |
| hEpCAM exp2 | 1.93E+06 | 2.57E−03 | 1.33E−09 | 1.66E+06 | 2.35E−03 | 1.42E−09 | 6.52E+05 | 3.45E−03 | 5.29E−09 |
| hEpCAM exp3 | 1.58E+06 | 2.54E−03 | 1.61E−09 | 1.62E+06 | 2.01E−03 | 1.24E−09 | 3.95E+05 | 3.30E−03 | 8.37E−09 |
| hEpCAM average | 1.81E+06 | 2.36E−03 | 1.30E−09 | 1.75E+06 | 2.13E−03 | 1.22E−09 | 5.12E+05 | 3.44E−03 | 6.73E−09 |
| cynoEpCAM exp1 | 9.38E+05 | 1.67E−03 | 1.78E−09 | 8.94E+05 | 2.28E−03 | 2.55E−09 | 2.99E+05 | 4.55E−03 | 1.52E−08 |
| cynoEpCAM exp2 | 8.28E+05 | 1.80E−03 | 2.17E−09 | 7.83E+05 | 2.66E−03 | 3.39E−09 | 4.54E+05 | 4.17E−03 | 9.18E−09 |
| cynoEpCAM exp3 | 8.64E+05 | 1.84E−03 | 2.12E−09 | 8.64E+05 | 0.002506 | 2.90E−09 | 3.44E+05 | 3.86E−03 | 1.12E−08 |
| cynoEpCAM average | 8.77E+05 | 1.77E−03 | 2.02E−09 | 8.47E+05 | 2.48E−03 | 2.93E−09 | 3.66E+05 | 4.19E−03 | 1.15E−08 |

TABLE 18
Affinity of BA3182 to human and cynomolgus CD3 at different pH conditions. The results from 3 independent experiments
and the average values are listed. Data calculated from a single analyte concentration are shaded in gray.

| | pH 6.0 | | | pH 6.5 | | | pH 7.4 | | |
|---|---|---|---|---|---|---|---|---|---|
| | kₐ [M · s] | k_{d}[s⁻¹] | K_{D}[M] | kₐ [M · s] | k_{d}[s⁻¹] | K_{D}[M] | kₐ [M · s] | k_{d}[s⁻¹] | K_{D}[M] |
| hCD3 exp1 | 1.74E+05 | 1.59E−03 | 9.16E−09 | 1.34E+05 | 1.19E−03 | 8.89E−09 | 7.92E+04 | 2.69E−03 | 3.39E−08 |
| hCD3 exp2 | 1.92E+05 | 1.43E−03 | 7.44E−09 | 1.23E+05 | 1.45E−03 | 1.18E−08 | 8.77E+04 | 3.00E−03 | 3.42E−08 |
| hCD3 exp3 | 1.62E+05 | 1.24E−03 | 7.66E−09 | 1.79E+05 | 1.33E−03 | 7.41E−09 | 8.72E+04 | 3.22E−03 | 3.69E−08 |
| hCD3 average | 1.76E+05 | 1.42E−03 | 8.08E−09 | 1.46E+05 | 1.32E−03 | 9.09E−09 | 8.47E+04 | 2.97E−03 | 3.50E−08 |
| cynoCD3 exp1 | 3.84E+05 | 3.87E−03 | 1.01E−08 | 2.80E+05 | 3.13E−03 | 1.12E−08 | 2.08E+05 | 6.29E−03 | 3.03E−08 |
| cynoCD3 exp2 | 3.75E+05 | 4.12E−03 | 1.10E−08 | 2.01E+05 | 3.05E−03 | 1.51E−08 | 2.29E+05 | 6.60E−03 | 2.88E−08 |
| cynoCD3 exp3 | 3.77E+05 | 4.52E−03 | 1.20E−08 | 2.26E+05 | 2.16E−03 | 9.55E−09 | 3.05E+05 | 8.37E−03 | 2.74E−08 |
| cynoCD3 average | 3.79E+05 | 4.17E−03 | 1.10E−08 | 2.36E+05 | 2.78E−03 | 1.18E−08 | 2.47E+05 | 7.09E−03 | 2.86E−08 |

### Example 26. Fcγ and FcRn Receptor Binding Analysis by SPR

This example shows the binding affinity (K_{D}) of BA3182 to human FcγRI, FcγRIIa, FcγIIb/c, FcγRIIIa, FcγRIIIb and FcRn receptors using surface plasmon resonance (SPR) technology. The Fc-gamma receptor extracellular domains were captured on the chip using an anti-His-tag antibody. Serial dilutions of BA3182 and a control IgG1 antibody were injected across the eight detection spots containing the Fc-gamma receptor and a control surface containing only the anti-His antibody. BA3182 has a non-glycosylated Fc-domain (N297Q mutation) and—as expected—did not bind to the Fc-gamma receptors. Binding to FcRn at pH 6.0 was measured by immobilizing BA3182 or a control IgG1 on the chip surface and injecting a serial dilution of FcRn. BA3182 showed similar binding affinity to FcRn as the IgG1 control antibody. The binding kinetics were calculated using a SICK Longmuir 1:1 model built into the analysis software.

### Materials

### Antibodies BA3182

IgG1-Ctrl (lot #13578) produced by Evitria (Zürich, Switzerland)

### Instruments and Reagents.

| Name | Vendor | Cat. No. | Lot No. |
|---|---|---|---|
| SPR32 Pro Instrument | Bruker | NA | NA |
| SPR High-Capacity Amine | Bruker | 1862615 | 21SPR09A |
| Sensors | | | |
| Flat Amine Chip | Bruker | SPR-AS-AMF | 10BB10215A |
| Amine Coupling Kit | Bruker | 1862634 | 10DA1313BA |
| Acetate 4.5 | Bruker | 1862646 | 10DA11285L |
| Acetate 5.5 | Bruker | 1862646 | 10DA11285N |
| Ethanolamine-HCl | Bruker | 1862634 | 10DA1133C |
| 10 × HBS-EP+ | GE | BR100669 | BCBR4629V |
| THE ™ His tag antibody | Genscript | A00186 | 14L000861 |
| Amicon Spin Column | EMD Millipore | UFC903024 | RIPB80727 |
| FcγRI/CD64 | ACRO | FCA-H52H1 | RC163P1-20BUF1-XF |
| FcγRIIa/CD32a (H131) | Sino Biological | 10374-H08C1 | LC13JU0607 |
| FcγRIIb/c (CD32b/c) | Sino Biological | 10259-H08H | LC15MY1709 |
| FcγRIIIa/CD16a (F158) | Sino Biological | 10389-H08H | LC15AU1812 |
| FcγRIIIb/CD16b | Sino Biological | 11046-H08C | LC15NO0404 |
| FcRn | ACRO | FCM-H5286 | A476-8CMF1-MA |

Methods The SPR32 Pro instrument has eight flow channels (1-8) with four detection spots each (A, B, C, D). The 32 detection spots can be addressed individually or in groups. The surface of each new sensor chip was preconditioned according to the manufacturer's recommendations using the built-in method before immobilization of proteins. PBST pH 7.4 was used as running buffer for the preconditioning and the immobilization of all antibodies.

All 32 spots on a preconditioned chip were activated by injecting a mixture 1-ethyl-3-(3-dimethylaminopropyl) carbodiimide/N-hydroxysuccinimide (EDC/NHS) (250 mM/50 mM) for 420 s at a flow rate of 10 μL/min. The His-Tag antibody was diluted to 50 μg/mL in NaAc pH 4.5 and injected over all 32 detection spots for 360 s at a flow rate of 10 μL/min. All 32 detection spots were blocked by injecting ethanolamine (1M) for 420 s at a flow rate of 10 μL/min.

For the binding test of BA3182 with the Fcγ receptors a capture assay format was used. The Fcγ receptor was diluted in running buffer and captured in spot B, C, or D of an anti-His sensor chip. Next BA3182 or the IgG1-ctrl antibody (dilution series in running buffer) was injected over AB, BC or CD spots. Detection spots without captured protein were used as control surface. Injections of running buffer were used as blanks. The His capture surface was regenerated after each analyte injection. One injection of the IgG1-ctrl antibody and three injections of BA3182 were performed for each assay.

### FcγRI (CD64) Capture Assay

Running buffer: Phosphate buffered saline containing Tween-20 (PBST), pH 7.4 FcγRI (CD64): 50 nM in PBST, pH 7.4, injected over D spots (flow rate: 10 L/min, contact time 15 s).
IgG1-ctrl: two-fold dilution series in PBST, pH 7.4 starting at 6 μg/mL, injected over CD spots (flow rate: 20 L/min, contact time 180 s; off-rate measurement 400 s).
BA3182: two-fold dilution series in PBST, pH 7.4, starting at 6 μg/mL, injected over CD spots (flow rate: 20 μL/min, contact time 180 s; off-rate measurement 400 s)

### FcγRIIa H131 (CD32a) Capture Assay

FcγRIIa H131 (CD32a): Running buffer was 181.8 nM in HBS-EP+, injected over C spots (flow rate: 10 L/min, contact time 60 s).
IgG1-ctrl: two-fold dilution series in HBS-EP+ starting at 1.5 mg/mL, injected over BC spots (flow rate: 20 L/min, contact time 60 s; off-rate measurement 60 s).
BA3182: two-fold dilution series in HBS-EP+, starting at 1.5 mg/mL, injected over BC spots (flow rate: 20 L/min, contact time 60 s; off-rate measurement 60 s).
Regeneration: 10 mM Glycine, pH 2.0, injected over BC spots (flow rate: 25 μL/min, contact time 15 s)

### FcγRIIb/c (CD32b/c) Capture Assay

Running buffer: HEPES/NaCl buffer containing EDTA and surfactant P20 (HBS-EP+) FcγRIIb/c (CD32b/c): 192.3 nM in HBS-EP+, injected over C spots (flow rate: 10 μL/min, contact time 60 s).
IgG1-ctrl: two-fold dilution series in HBS-EP+ starting at 1.5 mg/mL, injected over BC spots (flow rate: 20 μL/min, contact time 60 s; off-rate measurement 60 s).
BA3182: two-fold dilution series in HBS-EP+, starting at 1.5 mg/mL, injected over BC spots (flow rate: 20 μL/min, contact time 60 s; off-rate measurement 60 s).
Regeneration: 10 mM Glycine, pH 2.0, injected over BC spots (flow rate: 25 μL/min, contact time 15 s).

### FcγRIIIa F158 (CD16a) Capture Assay.

FcγRIIIa (CD16a): 171.7 nM in HBS-EP+, injected over C spots (flow rate: 10 μL/min, contact time 60s)
IgG1-ctrl: two-fold dilution series in HBS-EP+ starting at 1.5 mg/mL, injected over BC spots (flow rate: 20 L/min, contact time 60 s; off-rate measurement 60 s).
BA3182: two-fold dilution series in HBS-EP+, starting at 1.5 mg/mL, injected over BC spots (flow rate: 20 L/min, contact time 60 s; off-rate measurement 60 s).
Regeneration: 10 mM Glycine, pH2.0, injected over BC spots (flow rate: 25 μL/min, contact time 15 s).

### FcγRIIIb (CD16b) Capture Assay

FcγRIIIIb (CD16b): 180.2 nM in HBS-EP+, injected over C spots (flow rate: 10 L/min, contact time 60 s)
IgG1-ctrl: two-fold dilution series in HBS-EP+ starting at 1.5 mg/mL, injected over BC spots (flow rate: 20 μL/min, contact time 60 s; off-rate measurement 60 s).
BA3182: two-fold dilution series in HBS-EP+, starting at 1.5 mg/mL, injected over BC spots (flow rate: 20 L/min, contact time 60 s; off-rate measurement 60 s).
Regeneration: 10 mM Glycine, pH2.0, injected over BC spots (flow rate: 25 μL/min, contact time 15 s).

### FcRn Binding Assay.

BA3182 and IgG1-ctrl antibodies were immobilized on a flat amine sensor chip. All 32 spots on a preconditioned chip were activated by injecting a mixture EDC/NHS (250 mM/50 mM) for 420 s at a flow rate of 10 μL/min. Both BA3182 and the IgG1-ctrl antibody were diluted to 0.1 g/mL in NaAc pH 5.5 and injected over spots B(IgG1-ctrl) or C (BA3182) for 240 s at a flow rate of 10 L/min. All 32 detection spots were blocked by injecting Ethanolamine (1M) for 60 s at a flow rate of 10 μL/min.

FcRn: three-fold dilution series in PBS+TWEEN pH 6.0 starting at 25 μg/mL, injected over ABCD spots (flow rate: 20 μL/min, contact time 60 s; off-rate measurement 60 s). Regeneration: 1×PBS, pH 7.4, injected over ABCD spots (flow rate: 10 L/min, contact time 30s)

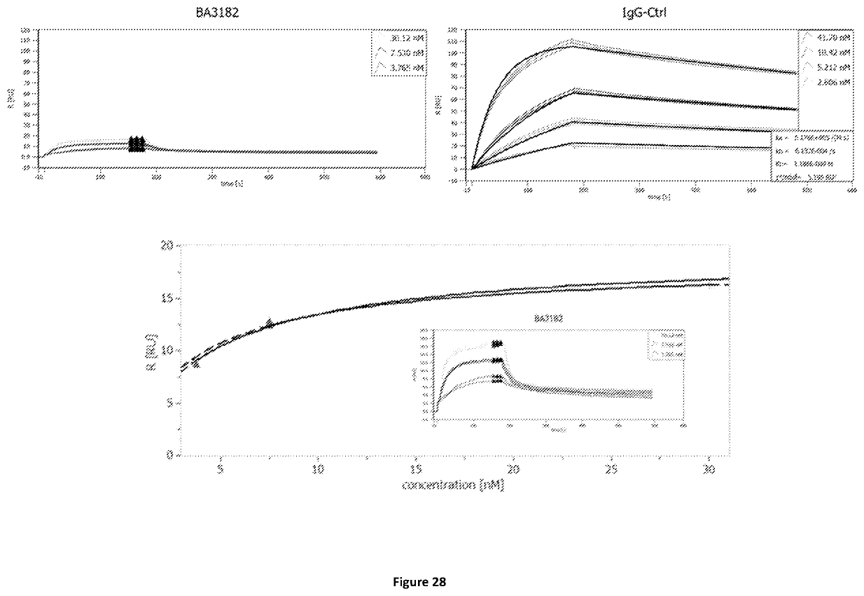

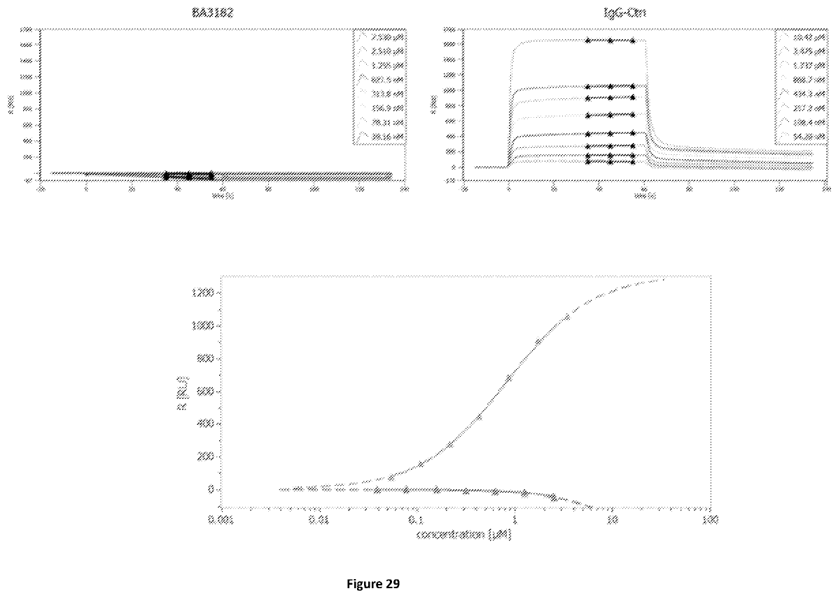

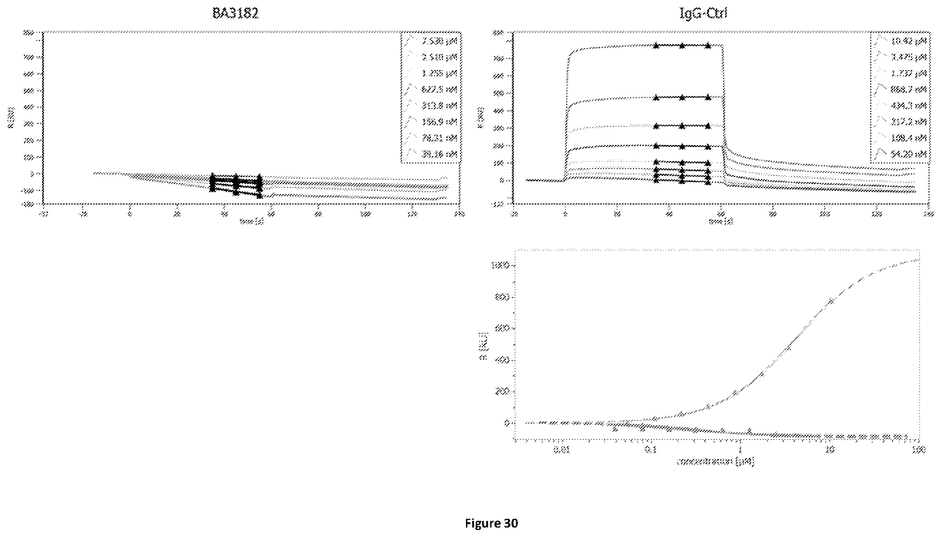

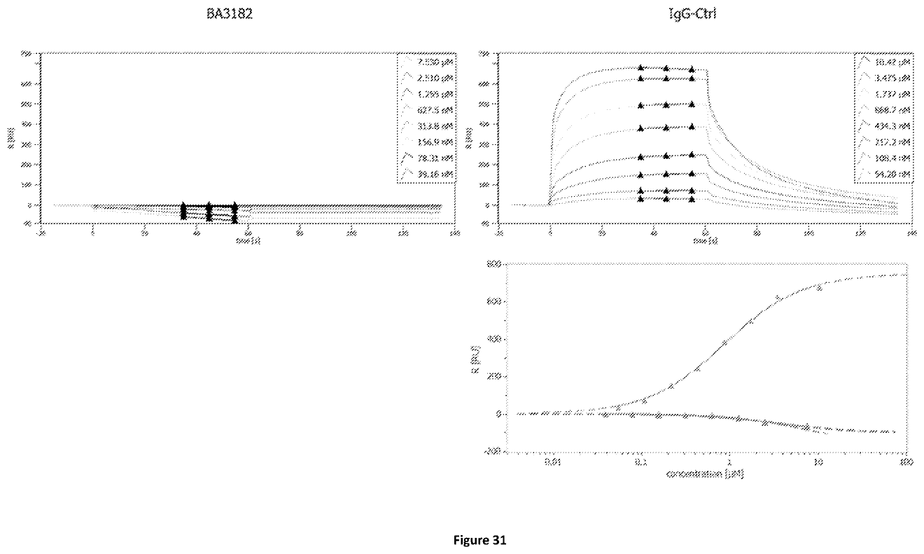

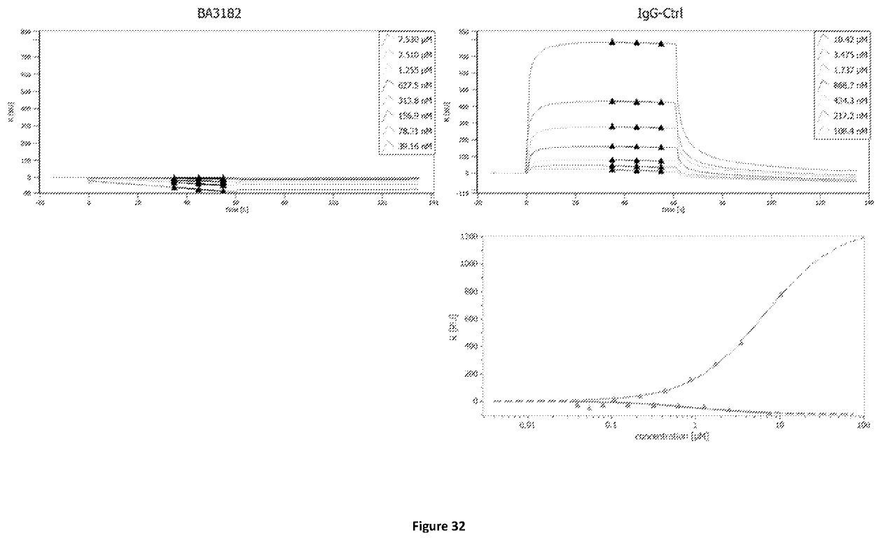

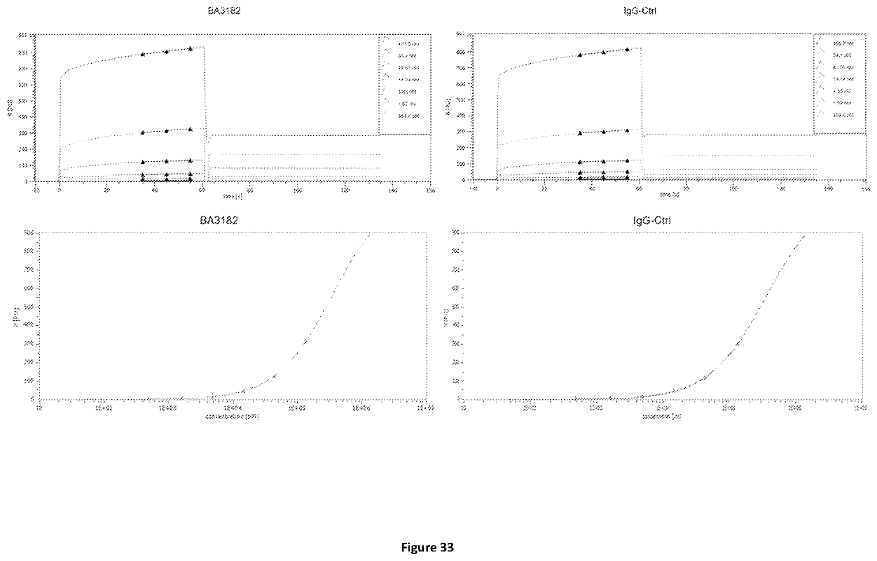

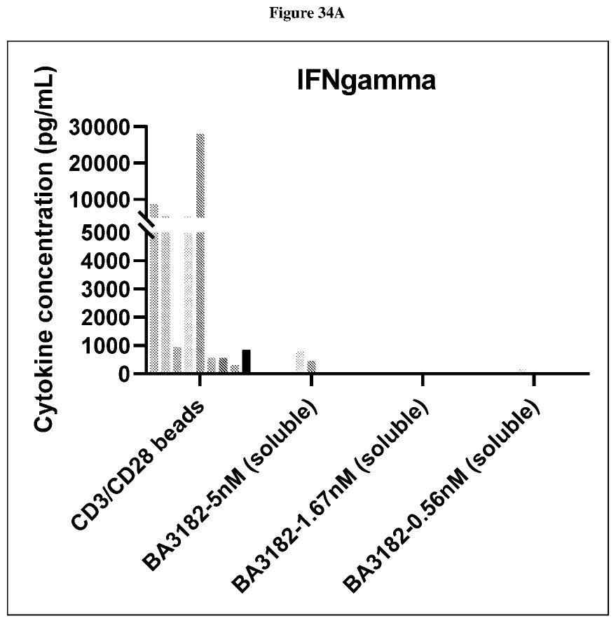

Results FIG. 28 shows that the IgG1-ctrl antibody with a wild-type human IgG1 Fc domain binds to FcγRI with an affinity of 1.18 nM. The binding signal for BA3182 was much lower and signal was detected only with the three highest concentrations tested. All binding curves reach saturation, so a steady state model was used to analyze the data. However, the signal intensity and difference between the different concentrations is too low to calculate a meaningful K_{D}. The data indicate that BA3182 does not bind to FcγRI (CD64). FIG. 29 shows that the IgG1-ctrl antibody with a wild-type human IgG1 Fc domain binds to FcγRIIa with an affinity of 0.82 μM. No binding was detected for BA3182 indicating that BA3182 does not interact with FcγRIIa (CD32a). FIG. 30 shows that the IgG1-ctrl antibody with a wild-type human IgG1 Fc domain binds to FcγRIIb/c with an affinity of 4.2 μM. No binding was detected for BA3182 indicating that BA3182 does not interact with FcγRIIb/c (CD32b/c). FIG. 31 shows that the IgG1-ctrl antibody with a wild-type human IgG1 Fc domain binds to FcγRIIIa with an affinity of 0.86 μM. No binding was detected for BA3182 indicating that BA3182 does not interact with FcγRIIIa (CD16a). FIG. 32 shows that the IgG1-ctrl antibody with a wild-type human IgG1 Fc domain binds to FcγRIIIa with an affinity of 0.86 μM. No binding was detected for BA3182 indicating that BA3182 does not interact with FcγRIIIa (CD16b). FIG. 33 shows that the IgG1-Ctrl antibody with a wild-type human IgG1 Fc domain binds to FcRn with an affinity of 366 nM. Similarly, BA3182 showed a binding affinity of 347 nM to FcRn.

### Example 27. Induced Cytokine Release Assay

The objective of this example was to determine if BA3182 can engage CD3 T cell activation in human PBMCs in the absence of EpCAM expressing cells. Human PBMCs were incubated with soluble or immobilized BA3182 for 48 hours. Soluble BA3182 was tested at 5 nM, 1.67 nM and 0.56 nM. The assay was carried out in culture media at pH 6.5 mimicking the pH of the tumor microenvironment, or at pH 7.4 mimicking the normal physiological pH. A panel of cytokines (IL-2, IL-4, IL-1P, IL-6, IL-10, IL-17, IFNγ, TNFα) were measured using a Luminex multiplex assay. Human T-activator, CD3/CD28 dynabeads for T cell expansion and activation were included as a positive control for human PBMCs cytokine response. A total of nine human PBMC donors were tested. No detectable levels of cytokines were observed upon stimulation of human PBMCs with soluble BA3182. IFNγ and TFNα were detected in cultures stimulated with immobilized BA3182, while IL-2, IL-4, IL-17, and IL-10 cytokines were detected at low levels in PBMC cultures from 3-4 human donors with immobilized BA3182. Immobilized OKT3 anti-CD3 antibody induced cytokine response in all human PBMC donors tested. The cytokine production induced by immobilized BA3182 was significantly lower at pH 7.4 than immobilized OKT3 anti-CD3 antibody. All cytokines were detectable in cultures stimulated with immobilized OKT3 anti-CD3 antibody except for IL-6, which was detected in PBMC cultures from two human donors only. All tested human PBMC donors showed elevated levels of cytokines upon activation with CD3/CD28 dynabeads regardless of the pH conditions. In general, the levels of cytokines induced by immobilized BA3182 were significantly lower in comparison to immobilized OKT3 anti-CD3 antibody. These results suggest that BA3182 induces poor cytokine response.

### Materials

### Antibodies

BA3182 (1.03 mg/mL)
Anti-hCD3 monoclonal antibody, clone OKT3: Tonbo, Cat #40-0037-U500,
Lot #P0037072518404
   PMBC cells.
Precision for Medicine, Cat #93000-10M, 9 donors, lot numbers 13143, 2010113371, 2010113397, 2010113292, 2010113411, 201675377, 201877535, 201885639, 201897529

### Reagents and Equipment

RPMI-1640: ThermoFisher Gibco Cat #11875-085, Lot #2463433
FBS: Sigma, Cat #12306C-500 mL, Lot #16J367
PBS: Gibco, Cat #10010-023, Lot #2430050
Cell culture grade water: Corning Cat #25-055-CM, lot #17216006
12N HCl: VWR, Cat #87003-251, Lot #4118020
50 mL conical tube: BioPioneer Cat #CNT-50, Lot #10272021
96 well clear flat bottom tissue culture plate: Corning, Cat #3596, Lot #19620012
Syringe filter: Celltreat Cat #229747, Lot #211004-052-1A
pH Meter: ORION STAR A111, ThermoFisher, serial #J17162
Trypan blue stain: Gibco Cat #15250-061, lot #1861515
Adhesive plate sealing films for microplate: E&K Scientific, Seal Plate, Cat #T396100 Lot #SG262G
Sample dilution block: Corning, Cat #3956, Lot #07718000
Dynabeads CD3/CD28: ThermoFisher, Cat #11131D, Lot #00637008
Luminex Multiplex cytokine kit: R&D systems, Cat #FCSTM03-8, Lot #1644401 & 1667482
Luminex LX200: Luminex, SN #LX10021050422
PBMC pH 6.5 assay media: RPMI-1640+10% FBS, adjust to pH 6.5 using 6N HCl, sterilize with 0.2 μm Syringe filter.
PBMC pH 7.4 assay media: RPMI-1640+10% FBS, adjust to pH 7.4 using 6N HCl, sterilize with 0.2 m Syringe filter.

### Methods

### Plate Coating

One day before the assay, coat the wells with 100 μL/well of anti-hCD3 antibody, clone OKT3 or BA3182 at 20 μg/mL in PBS buffer.
Cover with plate sealer and incubate at 4° C. overnight.

### PMBC Resting

Thaw the PBMCs in a water bath at 37° C. for 3 minutes and add to 10 mL warm PBMC assay media (pH 7.4) in a dropwise manner, swirl the conical tube constantly to help the cells adapt to the culture media temperature.
Pipette up and down using a 10 mL pipette until there are no visible clumps.
Collect the cells using a centrifuge at 1400 rpm for 10 minutes, discard the media supernatant.
Resuspend the cell pellet with 10 mL fresh warm media, and pipette to separate the cells. Incubate at 37° C., 5% CO₂ incubator overnight (between 18-20 hrs.).
On the assay day, determine cell number and viability using trypan blue stain solution. Collect the cells using a centrifuge at 1400 rpm for 10 minutes, discard the media supernatant.
Resuspend cells with designated volume of pH media to a density of 1×10⁶ cells/mL.

### Test Article Preparation and Assay Setup

Remove the BA3182 and anti-hCD3 antibody solution from the wells, and wash with 100 μL/well of PBS twice to remove unbound antibody.
Prepare the CD3/CD28 dynabeads according to the instructions by washing with at least 1 mL of PBS solution once. Resuspend to the appropriate volume with PBS solution, apply 2.5 μL beads per 1×10⁵ PBMC cells per well.
Prepare soluble BA3182 to the desired concentration at 5, 1.67 and 0.56 nM in pH 6.5 or pH 7.4 assay media.
Dispense 100 μL/well of the pH assay media to the BA3182 and anti-hCD3 coated wells. Dispense 100 μL/well of BA3182 to non-coated wells.
Add 100 L/well of the PBMC suspension prepared above. Final volume is 200 μL/well. Incubate at 37° C. with 5% CO₂ for 48 hrs.
Perform the cytokine multiplex assay following the kit manual using the culture supernatant, collect the data with Luminex LX100 unit.

Data Analysis Cytokine concentrations were determined using Luminex Multiplex cytokine kit from R&D systems and Luminex LX100 instrument from Luminex. A standard curve for each analyte was created by generating a five parameter logistic (5-PL) curve fit using xPonent software. Supernatants were diluted in assay diluent as required to obtain values in the linear range of the standard curve of the assay.

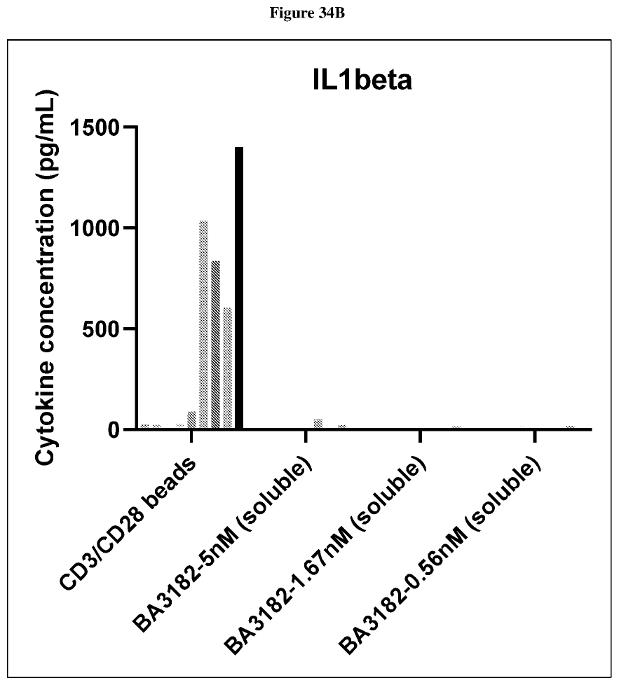

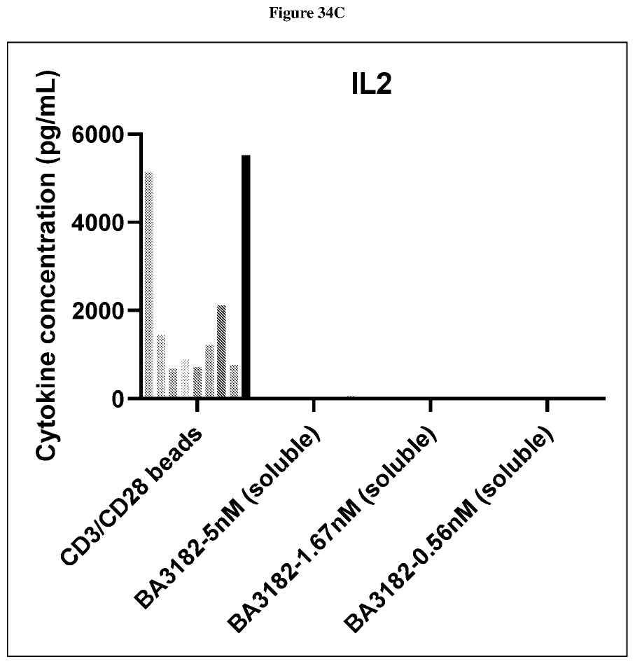

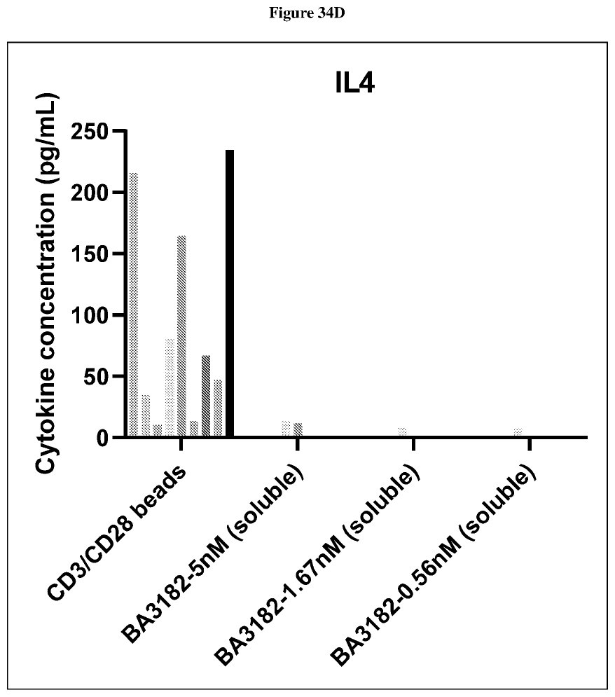

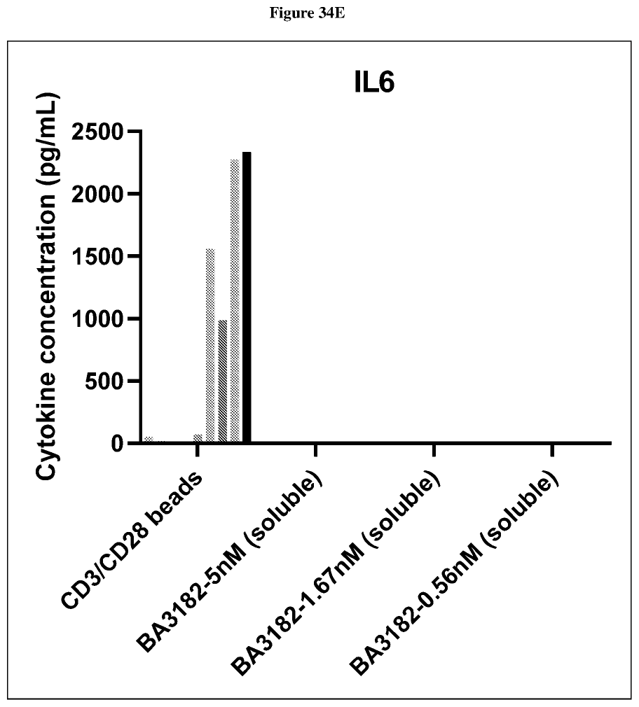

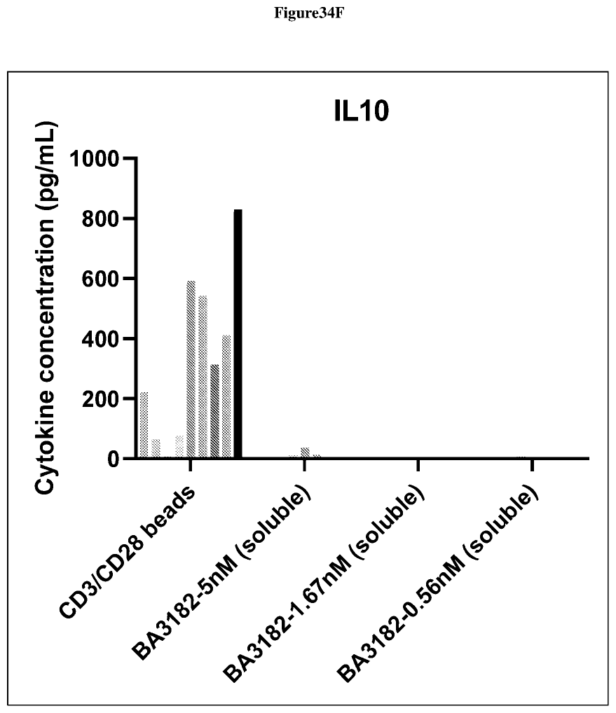

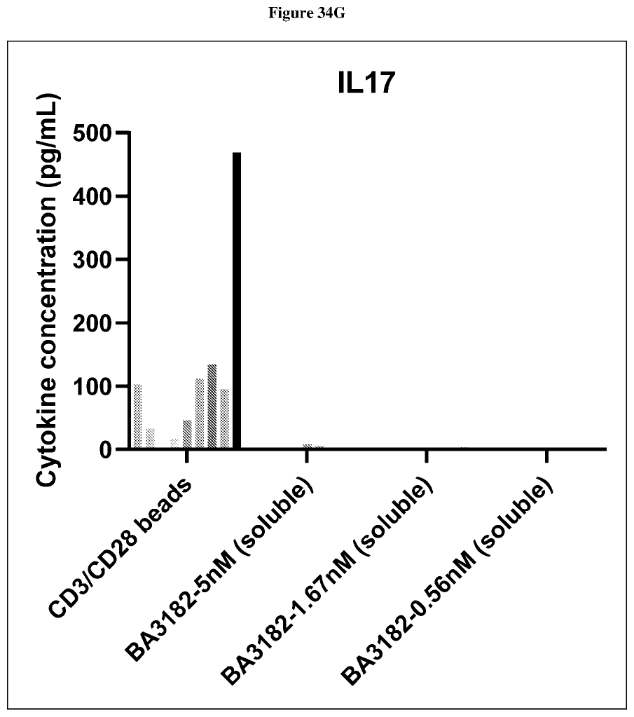

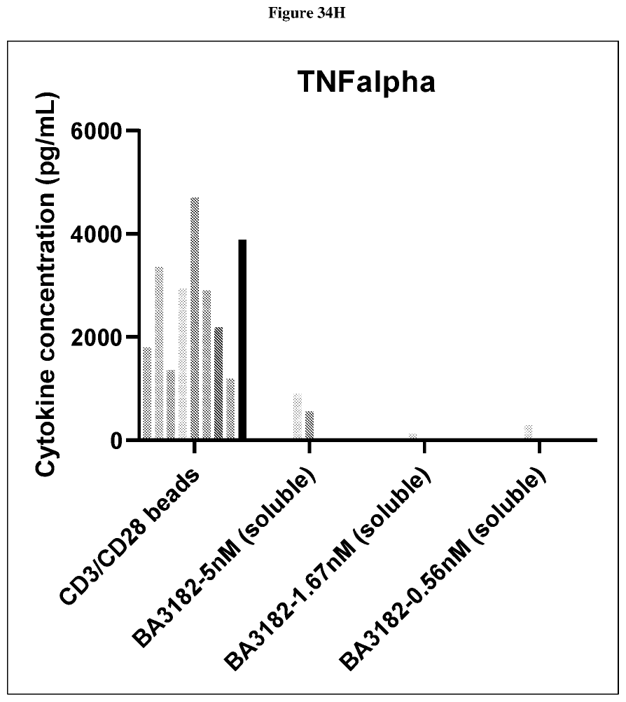

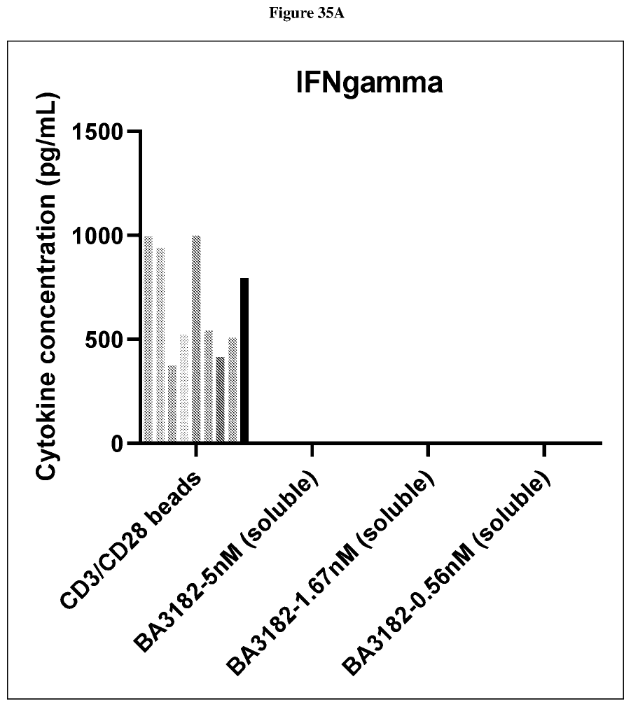

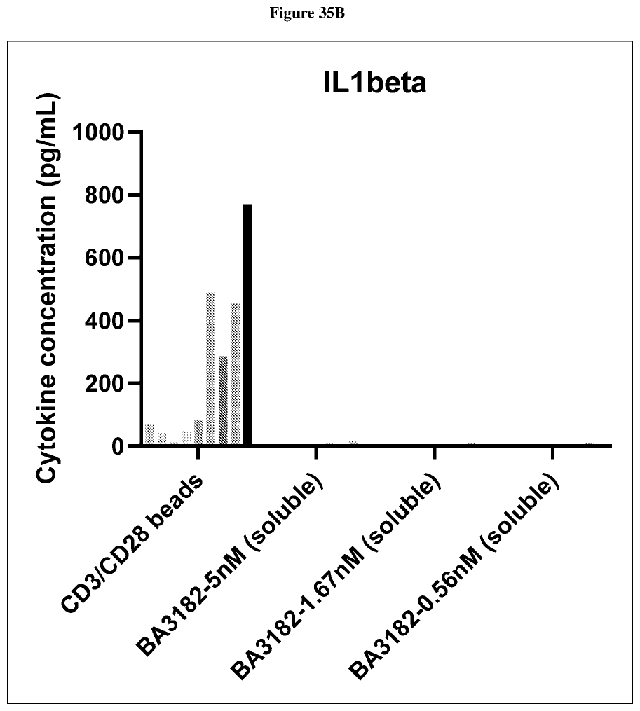

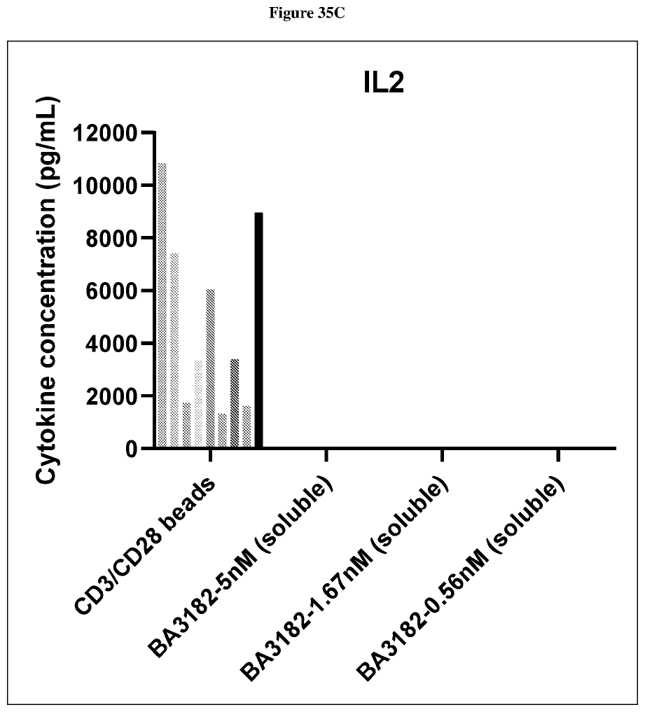

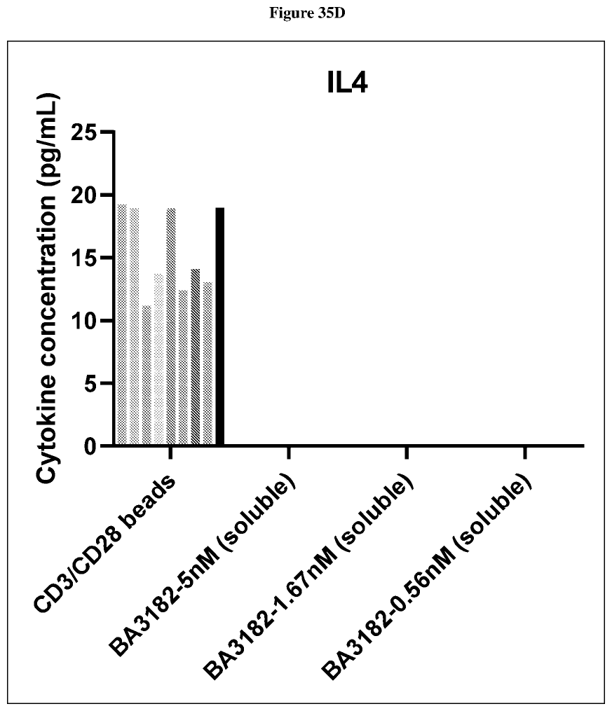

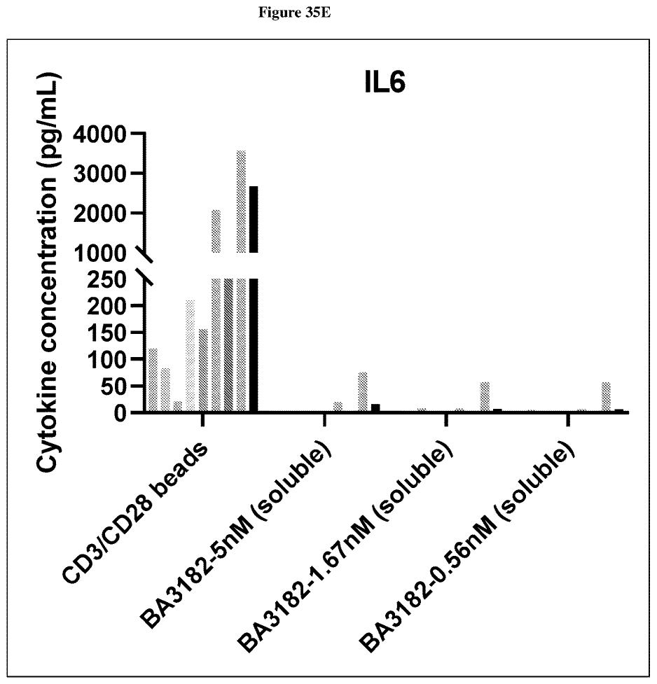

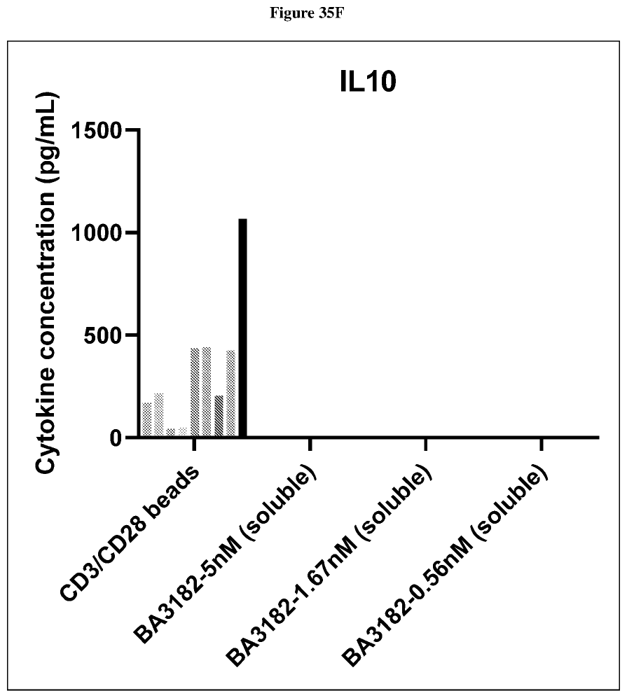

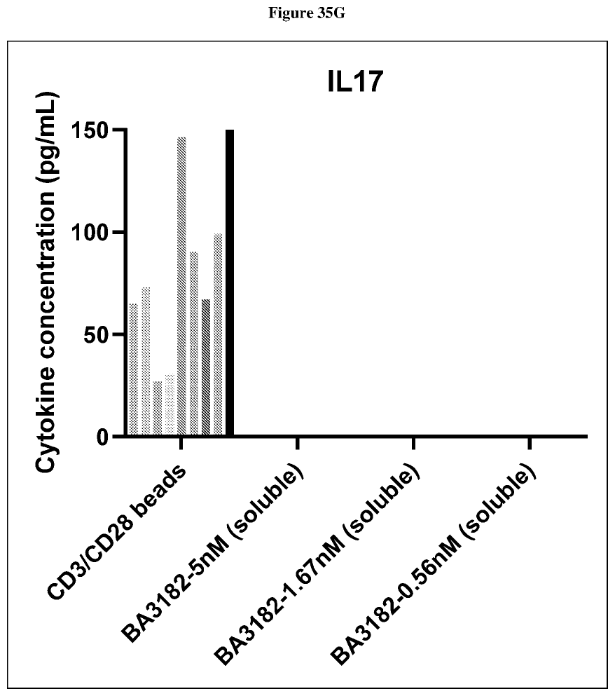

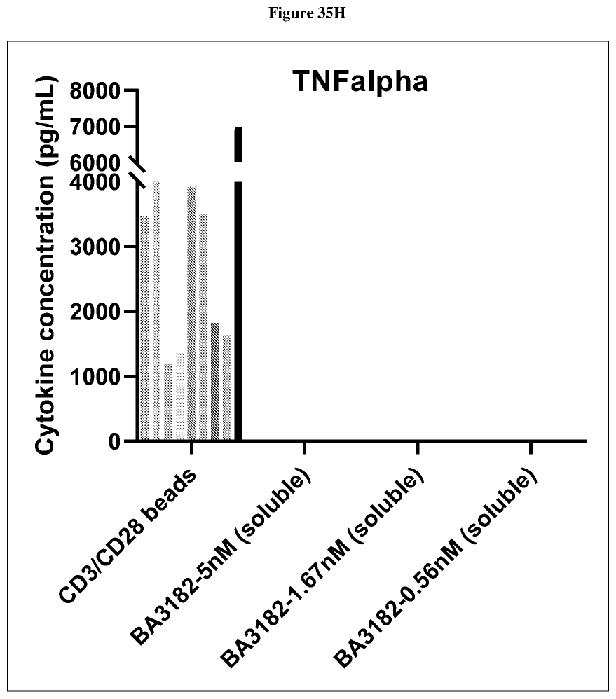

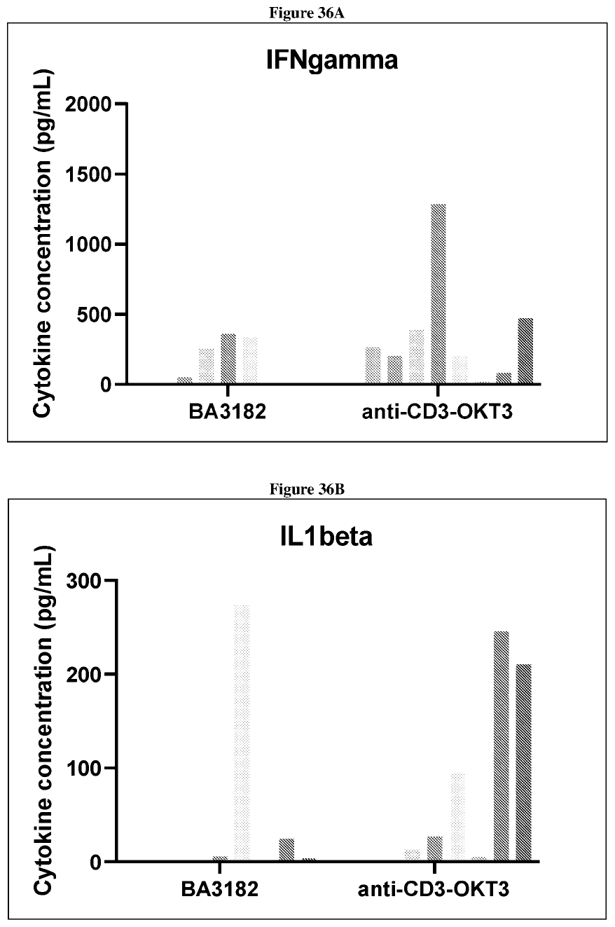

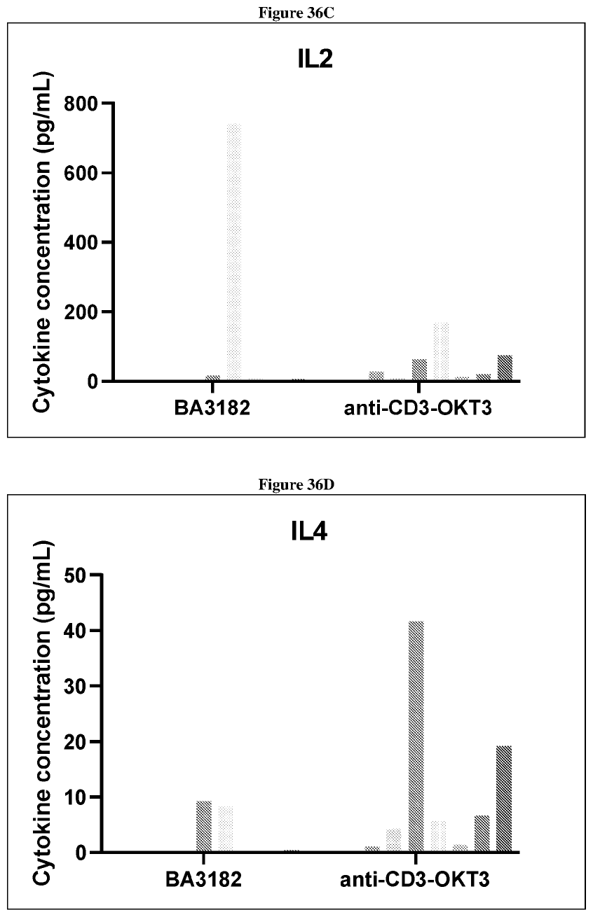

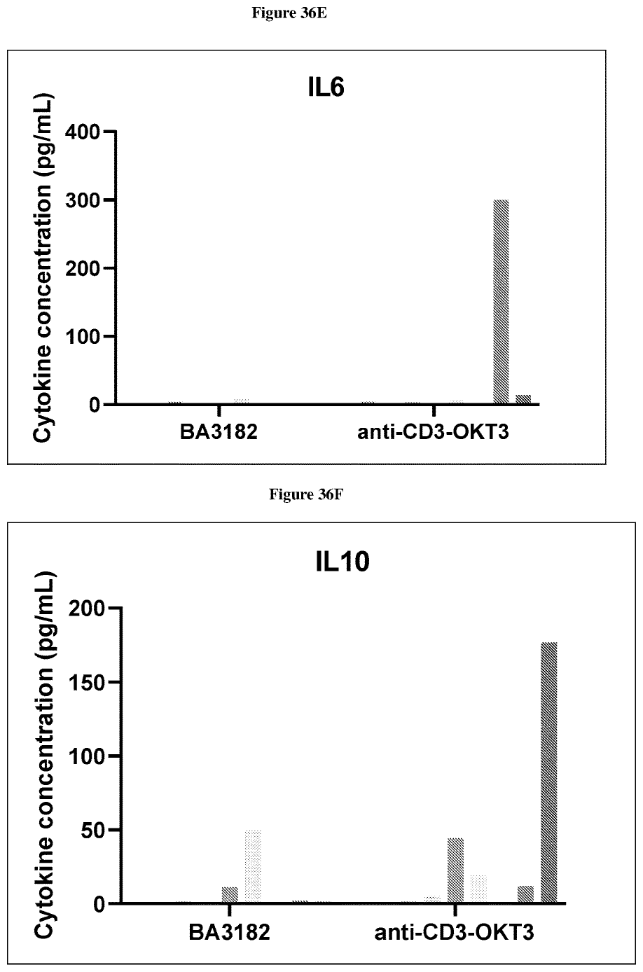

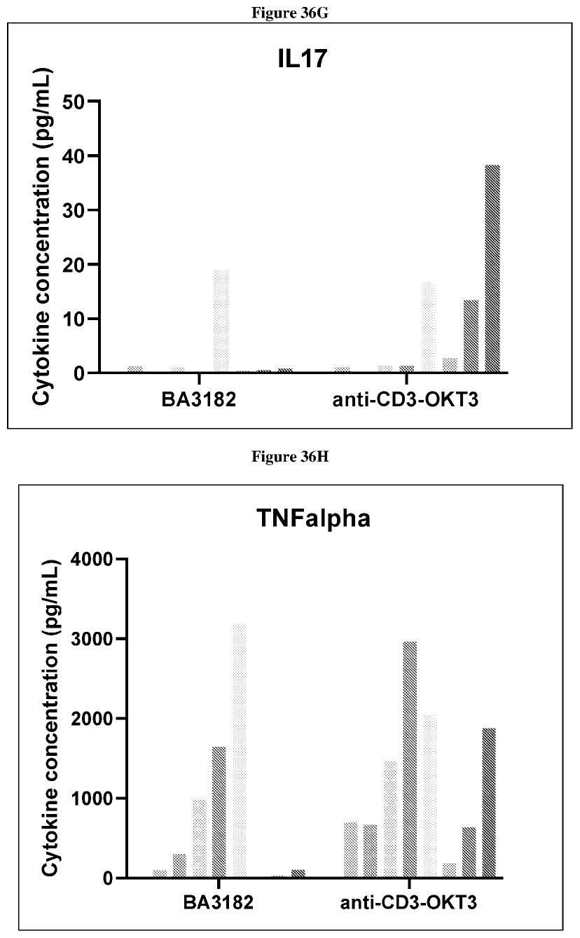

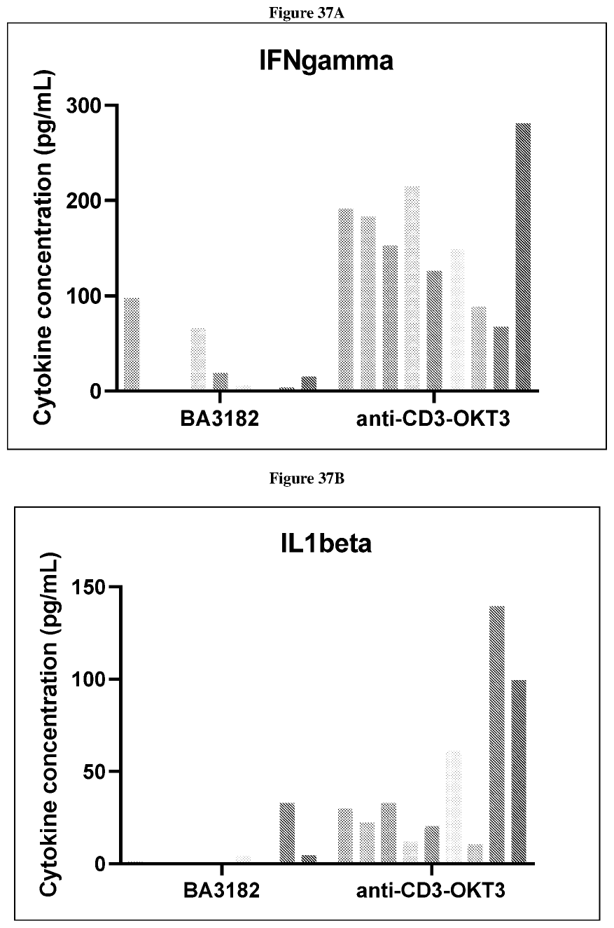

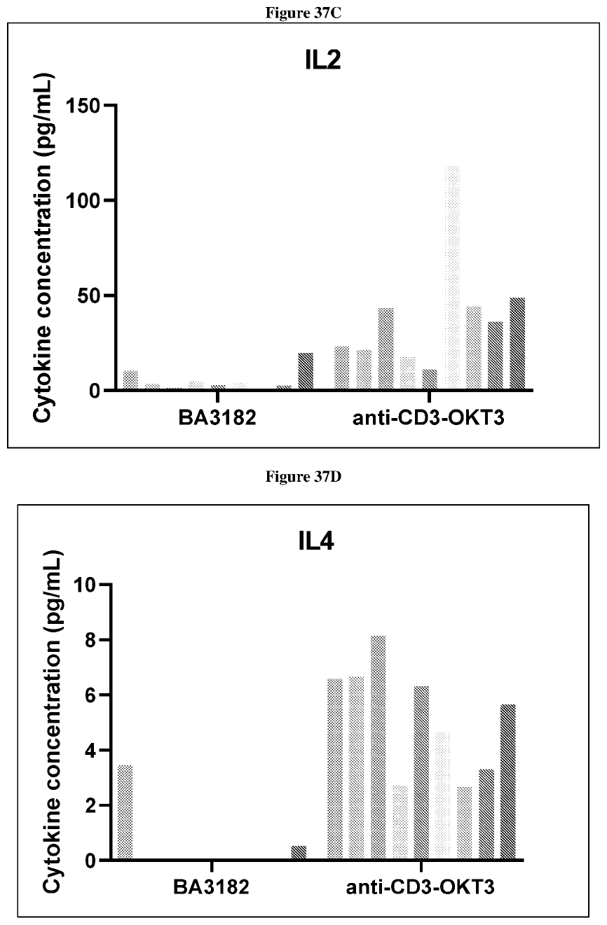

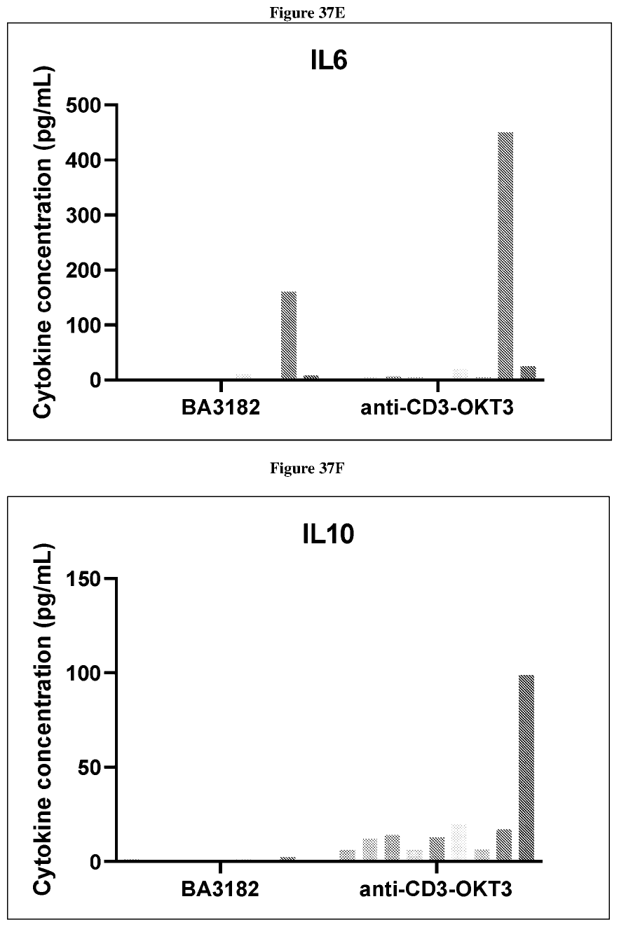

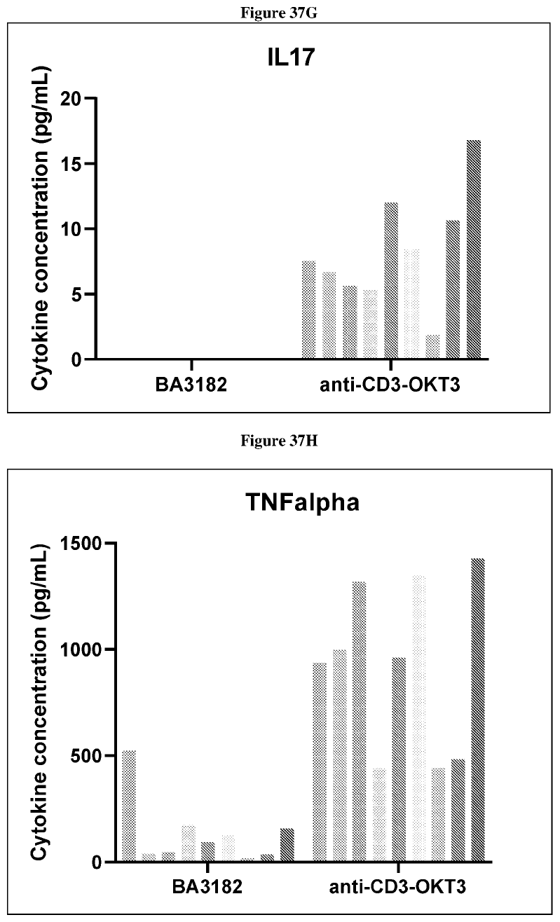

Results Soluble BA3182 did not induce detectable levels of any of the cytokines tested, including IFN-gamma, IL1-beta, IL-2, IL-4, IL-6, IL-10, IL-17, and TNF-alpha. The cytokine concentrations were either below the minimum detectable concentration range or similar to the levels of cytokines found in cultures of human PBMC in the absence of activating antibodies (FIGS. 34A-34H and FIGS. 35A-35H). Quantifiable levels of all cytokines were measured in supernatants from cultures of PBMCs activated with CD3/CD28 dynabeads. Cytokine levels were donor dependent. Immobilized BA3182 induced lower levels of cytokines in PBMC cultures at pH 6.5 and pH 7.4 in comparison to OKT3 anti-CD3 antibody. Among the cytokines tested, only IFN-gamma and TNF-alpha were detectable. Induction of IL-6 by immobilized BA3182 was only observed in one donor at pH 7.4, and it was significantly less than the levels induced by immobilized OKT3 anti-CD3 antibody. IL-1 beta, IL-4, IL-17 and IL-10 were induced by immobilized BA3182 in some cultures of human PBMCs at pH 6.5 and pH 7.4, and the levels of these cytokines were in general lower than the levels induced with immobilized OKT3 in PBMCs cultures from the same human donors. All cytokines tested were consistently detected in cultures stimulated with immobilized OKT3 anti-CD3 antibody except for IL-6, which was detectable in PBMC cultures from two human donors only (FIGS. 36A-36H and FIGS. 37A-37H). These results suggest that BA3182 induces poor cytokine response in the absence of EpCAM antigen.

### Example 28. Pharmacokinetic Analysis of Anti-EpCAM Bispecific Antibody Following Intravenous Administration to Female BALB/c Nude Mice

This example shows the pharmacokinetic analysis of BAP150.31-BF45. The mouse serum samples were collected at different time points after a single IV administration of either 1 or 10 mg/kg of BAP150.31-BF45 to female BALB/c nude mice. BAP150.31-BF45 concentrations in the collected serum samples were determined by affinity ELISA using recombinant human EpCAM coated plates to capture BAP150.31-BF45. The ELISA results showed that BAP150.31-BF45 concentrations in the serum increased along with the increased dosing of BAP150.31-BF45. Further descriptive PK parameters were established by pharmacokinetics analysis using the mean serum BAP150.31-BF45 concentration-time plotting. Overall, the systemic exposure for BAP150.31-BF45 increased with increasing BAP150.31-BF45 dose. The half-life for BAP150.31-BF45 in mouse serum was calculated to be 58.7 h based on the 1 mg/kg dose and 47 h based on the 10 mg/kg dose.

### Materials

### Antibody.

BAP150.31-BF45, 0.9 mg/mL

### Reagents.

Target antigen, human EpCAM-his protein, 2.485 mg/mL, diluted to 1 μg/mL with coating buffer.

### Anti-human IgG-HRP, Promega, cat #W403B, lot #0000297692.

Coating buffer, Sigma, cat #C3041-100CAP.
Mouse serum.
3,3′,5,5′ tetramethylbenzidine (TMB), Thermofisher, cat #002023, lot #03069141-7.
Bovine serum albumin (BSA), VWR, cat #332-25G, lot #20D0656194.

### Tween-20, Sigma, cat #P1379-500 mL, lot #SLBS7482.

### HCl, General-Reagent, cat #G81788B, lot #P1972715.

### Nunc™ MicroWell™ 96-Well Microplates, Thermofisher, cat #269787.

Assay buffer, PBS at pH 6.0 with 1% BSA.
Wash buffer, PBS at pH 6.0 with 0.05% Tween-20.
Standard Dilution Buffer, PBS at pH 6.0 with 1% BSA and 1:200 diluted normal mouse serum.

### Methods

### Standard

A 3-fold serial dilution of BAP150.31-BF45 in assay buffer at pH 6.0 was carried out for affinity ELISA assay. BAP150.31-BF45 standard concentrations ranged from 2830-0.05 ng/mL.

### ELISA Assay

Coat Nunc-Immuno MaxiSorp 96 well plates with 100 μL of 1 μg/mL of human
EpCAM-his antigen in coating buffer.
Cover with plate sealer and incubate at 4° C. overnight.
Empty plate and tap out residual liquid on paper towels.
Add 200 μL per well of assay buffer, shake at 200 RPM for 5 min at room temperature, then empty plate and tap out residual liquid on paper towels. Repeat wash step 3 times.
Add 200 μL per well of assay buffer.
Shake at 200 RPM for one hour at room temperature.
Empty plate and tape out residual liquid on paper towels.
Dilute each of the standard antibodies in standard dilution buffer to 2.83 μg/mL, then make 3-fold serial dilutions in standard dilution buffer. Dilute the test serum samples in PBS/l % BSA assay buffer accordingly to the layout.
Add 100 μL of the standard antibodies and test serum sample in each well following plate layout. Shake at 200 RPM for one hour at room temperature.
Empty plate and tap out residual liquid on paper towels. Add 200 μL per well of wash buffer, shake at 200 RPM for 5 min at room temp. Repeat wash step 3 times.
Add 100 μL per well of goat anti-human IgG-HRP, diluted 1:2500 in assay buffer.
Shake at 200 RPM for one hour at room temperature.
Empty plate and tap out residual liquid on paper towels. Add 200 μL per well of wash buffer. Repeat wash step 4 times.
Add 80 μL of TMB substrate solution per well.
Incubate at room temperature protected from light for certain development time: 3 min. Stop the enzymatic reaction by adding 80 μL 1N HCl.
Read OD at 450 nm using Multiskan™ Sky 51119770DP or equivalent.

Data Analysis Four parameter non-linear regression curves with variable slopes were generated by analysis of OD values obtained with the different concentrations of BAP150.31-BF45 using GraphPad Prism software. The concentrations of BAP150.31-BF45 in the mouse serum samples were determine by extrapolation from BAP150.31-BF45 standard curve. Non-compartment analysis of BAP150.31-BF45 plasma concentrations was conducted using PK Solver 2.0.

In Vivo Stability Assay BALB/c nude mice were randomly grouped into 2 groups containing 4 mice/group and administered intravenously with BAP150.31-BF45 at 1 and 10 mg/kg. The dosing volume was 10 mL/kg. Detailed information about animal grouping and sampling can be found in Table 19. The body weight of all animals was monitored throughout the study and no body weight loss or clinical signs were observed (Table 20). Serum samples from all animals at 10 min, 6, 24, 48, 96 and 168 hours post BAP150.31-BF45 administration were obtained and used for bioanalytical sample analysis.

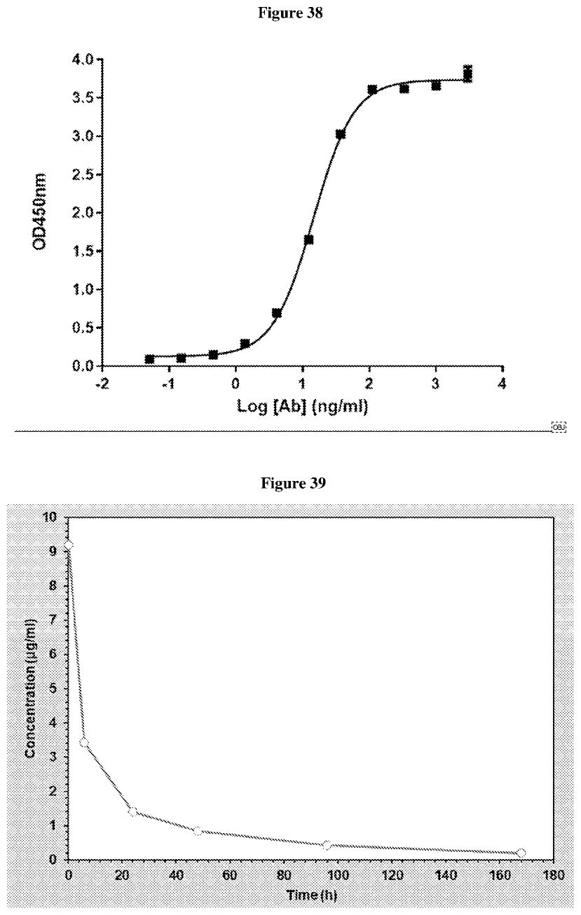

Affinity ELISA Assay The concentration of BAP150.31-BF45 in mouse serum samples from above was determined using enzyme-linked immunosorbent assay (ELISA). BAP150.31-BF45 standard concentrations in 0.5% normal mouse serum ranged from 2830-0.05 ng/mL; and the linear range of quantitation in 0.5% normal mouse serum is 104.81-1.29 ng/mL (FIG. 38). The mean concentrations of BAP150.31-BF45 in the serum samples are shown in Table 21. BAP150.31-BF45 was undetectable in pre-dosing serum samples. In treated animals, the concentrations of BAP150.31-BF45 in the serum increased with increasing doses of BAP150.31-BF45.

Pharmacokinetics Analysis Non-compartment analysis of BAP150.31-BF45 serum concentrations using intravenous bolus input was conducted using PK Solver 2.0. Serum samples with concentrations below the quantifiable limit (BQL) were assigned values of zero to compute the mean serum concentrations, and for the PK analysis (BQL<0.05 ng/mL).
Cₘₐₓ is the observed maximum serum concentration after dosing.
C₀ is the estimated concentration at time 0 h.
Tₘₐₓ is the time to reach the Cₘₐₓ.
AUC_{(0-T)} is the area under the plasma concentration-time curve from time zero to the last measurable time point calculated using linear trapezoidal rule.
AUC_{(0-inf)} is the area under the serum concentration-time curve from time zero to infinity.
CL is elimination clearance.
V_{Z} is volume of distribution during pseudo-equilibrium.
V_{SS} is volume of distribution at steady state.
T_{1/2} is terminal half-life results

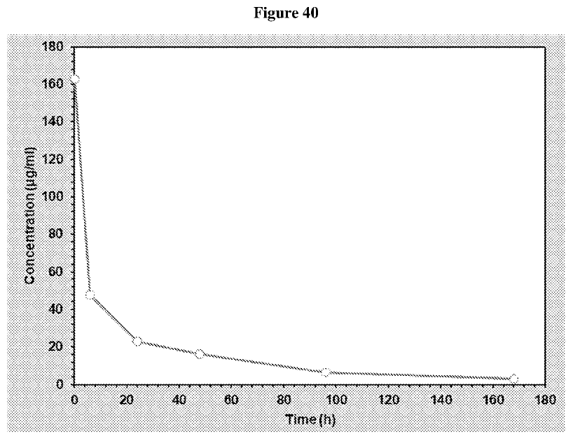

The serum PK parameters of BAP150.31-BF45 in mice are presented in Tables 22 and 23. Serum concentration-time profiles following a single dose of BAP150.31-BF45 in mice are shown in FIGS. 39 and 40. For group 1 (1 mg/kg), the Cₘₐₓ was 9.18 μg/mL, the AUC_{(0-T)} was 160.17 μg·h/mL and the AUC_{(0-inf)} was 176.96 μg·h/mL. For group 2 (10 mg/kg), the Cₘₐₓ was 162.62 μg/mL, the AUC_{(0-T)} was 2624 μg·h/mL and the AUC_{(0-inf)} was 2826.51 μg·h/mL. Overall, systemic exposure for BAP150.31-BF45 increased with increasing dose of BAP150.31-BF45. T_{1/2} values were generally similar between two dosed groups and were 58.72 and 47.99 h for doses of BAP150.31-BF45 at 1 and 10 mg/kg, respectively.

TABLE 19
Study groups, animal identification number,
dosing volumes and sampling time points.

| Group # | Animal ID | Sampling time points |
|---|---|---|
| Group-1 | 73134 | 10 min, 6, 24, 48, 96 and 168 h |
| BA150.31-BF45, 1 mg/kg, | 73158 | |
| IV, 10 μL/g | 73171 | |
| 1 dose | 73179 | |
| Group-2 | 73131 | |
| BA150.31-BF45, 10 mg/kg, | 73142 | |
| IV, 11.11 μL/g | 73147 | |
| 1 dose | 73169 | |

TABLE 20
Animal body weights at the beginning and at the end of the study.

Body weight (g)

| Group # | Animal No. | Day 0 | Day 6 |
|---|---|---|---|
| Group-1 | 73134 | 20.96 | 22.42 |
| BA150.31-BF45, 1 mg/kg | 73158 | 20.65 | 21.41 |
| | 73171 | 21.35 | 21.18 |
| | 73179 | 18.06 | 20.75 |
| Group-2 | 73131 | 21.36 | 23.12 |
| BA150.31-BF45, 10 mg/kg | 73142 | 18.72 | 20.08 |
| | 73147 | 19.83 | 21.12 |
| | 73169 | 19.89 | 21.59 |

TABLE 21
The average concentrations of BAP150.31-BF45 in mouse serum samples.
BAP150.31-BF45 in serum (ug/mL)

| Time (hr) | 1 mg/kg | 10 mg/kg |
|---|---|---|
| 0.17 | 9.18 | 162.62 |
| 6 | 3.41 | 47.70 |
| 24 | 1.39 | 22.82 |
| 48 | 0.83 | 15.98 |
| 96 | 0.42 | 6.56 |
| 168 | 0.20 | 2.92 |

TABLE 22
Serum PK parameters for BAP150.31-BF45 following a
single 1 mg/kg IV administration of BAP150.31-BF45.

| Parameter | Unit | Value |
|---|---|---|
| T½ | h | 58.72 |
| Tmax | h | 0.17 |
| Tlast | h | 168.00 |
| Cmax | μg/ml | 9.18 |
| C0 | μg/ml | 9.45 |
| AUC 0-t | μg/ml*h | 160.17 |
| AUC 0-inf_obs | μg/ml*h | 176.96 |
| Vz_obs | (mg/kg)/(μg/ml) | 0.48 |
| Cl_obs | (mg/kg)/(μg/ml)/h | 0.01 |
| Vss_obs | (mg/kg)/(μg/ml) | 0.33 |

TABLE 23
Serum PK parameters for BAP150.31-BF45 following a single
10 mg/kg IV administration of BAP150.31-BF45.

| Parameter | Unit | Value |
|---|---|---|
| T½ | h | 47.99 |
| Tmax | h | 0.17 |
| Tlast | h | 168.00 |
| Cmax | μg/ml | 162.62 |
| C0 | μg/ml | 168.54 |
| AUC 0-t | μg/ml*h | 2624.00 |
| AUC 0-inf_obs | μg/ml*h | 2826.51 |
| Vz_obs | (mg/kg)/(μg/ml) | 0.24 |
| Cl_obs | (mg/kg)/(μg/ml)/h | 0.00 |
| Vss_obs | (mg/kg)/(μg/ml) | 0.18 |

SEQUENCES

| | |
|---|---|
| SEQ ID NO: 1 | SASSSISYMH |
| SEQ ID NO: 2 | STSNLAS |
| SEQ ID NO: 3 | HQWSTYHT |
| SEQ ID NO: 4 | GYTFTSYWMH |
| SEQ ID NO: 5 | YIRPSTGYTEYNQKFKD |
| SEQ ID NO: 6 | GDNWVGFAN |
| SEQ ID NO: 7 | SASSSISDMH |
| SEQ ID NO: 8 | SASSSISEMH |
| SEQ ID NO: 9 | SASSSISYPH |
| SEQ ID NO: 10 | SASSSISYMI |
| SEQ ID NO: 11 | DTSNLAS |
| SEQ ID NO: 12 | ETSNLAS |
| SEQ ID NO: 13 | HTSNLAS |
| SEQ ID NO: 14 | KTSNLAS |
| SEQ ID NO: 15 | QTSNLAS |
| SEQ ID NO: 16 | SNSNLAS |
| SEQ ID NO: 17 | STSELAS |
| SEQ ID NO: 18 | STSNDAS |
| SEQ ID NO: 19 | STSNLKS |
| SEQ ID NO: 20 | STSNLAE |
| SEQ ID NO: 21 | EQWSTYHT |
| SEQ ID NO: 22 | HGWSTYHT |
| SEQ ID NO: 23 | HQWSTYKT |
| SEQ ID NO: 24 | GYNFTSYWMH |
| SEQ ID NO: 25 | GYTETSYWMH |
| SEQ ID NO: 26 | GYTHTSYWMH |
| SEQ ID NO: 27 | GYTKTSYWMH |
| SEQ ID NO: 28 | GYTFTSYHMH |
| SEQ ID NO: 29 | GYTFTSYWDH |
| SEQ ID NO: 30 | GYTFTSYWEH |
| SEQ ID NO: 31 | GYTFTSYWMW |
| SEQ ID NO: 32 | HIRPSTGYTEYNQKFKD |
| SEQ ID NO: 33 | YDRPSTGYTEYNQKFKD |
| SEQ ID NO: 34 | YERPSTGYTEYNQKFKD |
| SEQ ID NO: 35 | YIGPSTGYTEYNQKFKD |
| SEQ ID NO: 36 | YIHPSTGYTEYNQKFKD |
| SEQ ID NO: 37 | YIRLSTGYTEYNQKFKD |
| SEQ ID NO: 38 | YIRWSTGYTEYNQKFKD |
| SEQ ID NO: 39 | ERGDNWVGFAN |
| SEQ ID NO: 40 | WGDNWVGFAN |
| SEQ ID NO: 41 | YGDNWVGFAN |
| SEQ ID NO: 42 | GQNWVGFAN |
| SEQ ID NO: 43 | GDNWKGFAN |
| SEQ ID NO: 44 | GDNWVGMAN |
| SEQ ID NO: 45 | GDNWVGFKN |
| SEQ ID NO: 46 | GDNWVGFRN |
| SEQ ID NO: 47 | GDNWVGFAD |
| SEQ ID NO: 48 | GDNWVGFAE |
| SEQ ID NO: 49 | GDNWVGFAH |

SEQ ID NO: 50
GDNWVGFANR
SEQ ID NO: 51
DIQMTQSPSSLSASVGDRVTITCSASSSISYMHWYQQKPGQAPRLLIYSTSNLASGVPS
RFSGSGSGTDFTFTISSLEAEDAATYYCHQWSTYHTFGQGTKVEIK
SEQ ID NO: 52
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGRGDNWVGFANWGQ
GTLVTVSS
SEQ ID NO: 53
DIQMTQSPSSLSASVGDRVTITCSASSSISDMHWYQQKPGQAPRLLIYSTSNLASGVPS
RFSGSGSGTDFTFTISSLEAEDAATYYCHQWSTYHTFGQGTKVEIK
SEQ ID NO: 54
DIQMTQSPSSLSASVGDRVTITCSASSSISEMHWYQQKPGQAPRLLIYSTSNLASGVPS
RFSGSGSGTDFTFTISSLEAEDAATYYCHQWSTYHTFGQGTKVEIK
SEQ ID NO: 55
DIQMTQSPSSLSASVGDRVTITCSASSSISYPHWYQQKPGQAPRLLIYSTSNLASGVPS
RFSGSGSGTDFTFTISSLEAEDAATYYCHQWSTYHTFGQGTKVEIK
SEQ ID NO: 56
DIQMTQSPSSLSASVGDRVTITCSASSSISYMIWYQQKPGQAPRLLIYSTSNLASGVPS
RFSGSGSGTDFTFTISSLEAEDAATYYCHQWSTYHTFGQGTKVEIK
SEQ ID NO: 57
DIQMTQSPSSLSASVGDRVTITCSASSSISYMHWYQQKPGQAPRLLIYDTSNLASGVP
SRFSGSGSGTDFTFTISSLEAEDAATYYCHQWSTYHTFGQGTKVEIK
SEQ ID NO: 58
DIQMTQSPSSLSASVGDRVTITCSASSSISYMHWYQQKPGQAPRLLIYETSNLASGVP
SRFSGSGSGTDFTFTISSLEAEDAATYYCHQWSTYHTFGQGTKVEIK
SEQ ID NO: 59
DIQMTQSPSSLSASVGDRVTITCSASSSISYMHWYQQKPGQAPRLLIYHTSNLASGVP
SRFSGSGSGTDFTFTISSLEAEDAATYYCHQWSTYHTFGQGTKVEIK
SEQ ID NO: 60
DIQMTQSPSSLSASVGDRVTITCSASSSISYMHWYQQKPGQAPRLLIYKTSNLASGVP
SRFSGSGSGTDFTFTISSLEAEDAATYYCHQWSTYHTFGQGTKVEIK
SEQ ID NO: 61
DIQMTQSPSSLSASVGDRVTITCSASSSISYMHWYQQKPGQAPRLLIYQTSNLASGVP
SRFSGSGSGTDFTFTISSLEAEDAATYYCHQWSTYHTFGQGTKVEIK
SEQ ID NO: 62
DIQMTQSPSSLSASVGDRVTITCSASSSISYMHWYQQKPGQAPRLLIYSNSNLASGVP
SRFSGSGSGTDFTFTISSLEAEDAATYYCHQWSTYHTFGQGTKVEIK
SEQ ID NO: 63
DIQMTQSPSSLSASVGDRVTITCSASSSISYMHWYQQKPGQAPRLLIYSTSELASGVPS
RFSGSGSGTDFTFTISSLEAEDAATYYCHQWSTYHTFGQGTKVEIK
SEQ ID NO: 64
DIQMTQSPSSLSASVGDRVTITCSASSSISYMHWYQQKPGQAPRLLIYSTSNDASGVP
SRFSGSGSGTDFTFTISSLEAEDAATYYCHQWSTYHTFGQGTKVEIK
SEQ ID NO: 65
DIQMTQSPSSLSASVGDRVTITCSASSSISYMHWYQQKPGQAPRLLIYSTSNLKSGVPS
RFSGSGSGTDFTFTISSLEAEDAATYYCHQWSTYHTFGQGTKVEIK
SEQ ID NO: 66
DIQMTQSPSSLSASVGDRVTITCSASSSISYMHWYQQKPGQAPRLLIYSTSNLAEGVP
SRFSGSGSGTDFTFTISSLEAEDAATYYCHQWSTYHTFGQGTKVEIK
SEQ ID NO: 67
DIQMTQSPSSLSASVGDRVTITCSASSSISYMHWYQQKPGQAPRLLIYSTSNLASGVPS
RFSGSGSGTDFTFTISSLEAEDAATYYCEQWSTYHTFGQGTKVEIK
SEQ ID NO: 68
DIQMTQSPSSLSASVGDRVTITCSASSSISYMHWYQQKPGQAPRLLIYSTSNLASGVPS
RFSGSGSGTDFTFTISSLEAEDAATYYCHGWSTYHTFGQGTKVEIK
SEQ ID NO: 69
DIQMTQSPSSLSASVGDRVTITCSASSSISYMHWYQQKPGQAPRLLIYSTSNLASGVPS
RFSGSGSGTDFTFTISSLEAEDAATYYCHQWSTYKTFGQGTKVEIK
SEQ ID NO: 70
EVQLVESGGGLVQPGGSLRLSCAASGYNFTSYWMHWIRQSPSRGLEWLGYIRPSTG
YTEYNQKFKDRFTISRDDSKNTAYLQMNSLKTEDTAVYYCGRGDNWVGFANWGQ
GTLVTVSS
SEQ ID NO: 71
EVQLVESGGGLVQPGGSLRLSCAASGYTETSYWMHWIRQSPSRGLEWLGYIRPSTG
YTEYNQKFKDRFTISRDDSKNTAYLQMNSLKTEDTAVYYCGRGDNWVGFANWGQ
GTLVTVSS
SEQ ID NO: 72
EVQLVESGGGLVQPGGSLRLSCAASGYTHTSYWMHWIRQSPSRGLEWLGYIRPSTG
YTEYNQKFKDRFTISRDDSKNTAYLQMNSLKTEDTAVYYCGRGDNWVGFANWGQ
GTLVTVSS
SEQ ID NO: 73
EVQLVESGGGLVQPGGSLRLSCAASGYTKTSYWMHWIRQSPSRGLEWLGYIRPSTG
YTEYNQKFKDRFTISRDDSKNTAYLQMNSLKTEDTAVYYCGRGDNWVGFANWGQ
GTLVTVSS
SEQ ID NO: 74
EVQLVESGGGLVQPGGSLRLSCAASGYTFTSYHMHWIRQSPSRGLEWLGYIRPSTGY
TEYNQKFKDRFTISRDDSKNTAYLQMNSLKTEDTAVYYCGRGDNWVGFANWGQG
TLVTVSS
SEQ ID NO: 75
EVQLVESGGGLVQPGGSLRLSCAASGYTFTSYWDHWIRQSPSRGLEWLGYIRPSTGY
TEYNQKFKDRFTISRDDSKNTAYLQMNSLKTEDTAVYYCGRGDNWVGFANWGQG
TLVTVSS
SEQ ID NO: 76
EVQLVESGGGLVQPGGSLRLSCAASGYTFTSYWEHWIRQSPSRGLEWLGYIRPSTGY
TEYNQKFKDRFTISRDDSKNTAYLQMNSLKTEDTAVYYCGRGDNWVGFANWGQG
TLVTVSS
SEQ ID NO: 77
EVQLVESGGGLVQPGGSLRLSCAASGYTFTSYWMWWIRQSPSRGLEWLGYIRPSTG
YTEYNQKFKDRFTISRDDSKNTAYLQMNSLKTEDTAVYYCGRGDNWVGFANWGQ
GTLVTVSS
SEQ ID NO: 78
EVQLVESGGGLVQPGGSLRLSCAASGYTFTSYWMHWIRQSPSRGLEWLGHIRPSTG
YTEYNQKFKDRFTISRDDSKNTAYLQMNSLKTEDTAVYYCGRGDNWVGFANWGQ
GTLVTVSS
SEQ ID NO: 79
EVQLVESGGGLVQPGGSLRLSCAASGYTFTSYWMHWIRQSPSRGLEWLGYDRPSTG
YTEYNQKFKDRFTISRDDSKNTAYLQMNSLKTEDTAVYYCGRGDNWVGFANWGQ
GTLVTVSS
SEQ ID NO: 80
EVQLVESGGGLVQPGGSLRLSCAASGYTFTSYWMHWIRQSPSRGLEWLGYERPSTG
YTEYNQKFKDRFTISRDDSKNTAYLQMNSLKTEDTAVYYCGRGDNWVGFANWGQ
GTLVTVSS
SEQ ID NO: 81
EVQLVESGGGLVQPGGSLRLSCAASGYTFTSYWMHWIRQSPSRGLEWLGYIGPSTG
YTEYNQKFKDRFTISRDDSKNTAYLQMNSLKTEDTAVYYCGRGDNWVGFANWGQ
GTLVTVSS
SEQ ID NO: 82
EVQLVESGGGLVQPGGSLRLSCAASGYTFTSYWMHWIRQSPSRGLEWLGYIHPSTG
YTEYNQKFKDRFTISRDDSKNTAYLQMNSLKTEDTAVYYCGRGDNWVGFANWGQ
GTLVTVSS
SEQ ID NO: 83
EVQLVESGGGLVQPGGSLRLSCAASGYTFTSYWMHWIRQSPSRGLEWLGYIRLSTG
YTEYNQKFKDRFTISRDDSKNTAYLQMNSLKTEDTAVYYCGRGDNWVGFANWGQ
GTLVTVSS
SEQ ID NO: 84
EVQLVESGGGLVQPGGSLRLSCAASGYTFTSYWMHWIRQSPSRGLEWLGYIRWSTG
YTEYNQKFKDRFTISRDDSKNTAYLQMNSLKTEDTAVYYCGRGDNWVGFANWGQ
GTLVTVSS
SEQ ID NO: 85
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCERGDNWVGFANWGQ
GTLVTVSS
SEQ ID NO: 86
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGWGDNWVGFANWGQ
GTLVTVSS
SEQ ID NO: 87
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGYGDNWVGFANWGQ
GTLVTVSS
SEQ ID NO: 88
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGRGQNWVGFANWGQ
GTLVTVSS
SEQ ID NO: 89
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGRGDNWKGFANWGQ
GTLVTVSS
SEQ ID NO: 90
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGRGDNWVGMANWGQ
GTLVTVSS
SEQ ID NO: 91
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGRGDNWVGFKNWGQ
GTLVTVSS
SEQ ID NO: 92
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGRGDNWVGFRNWGQ
GTLVTVSS
SEQ ID NO: 93
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGRGDNWVGFADWGQ
GTLVTVSS
SEQ ID NO: 94
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGRGDNWVGFAEWGQ
GTLVTVSS
SEQ ID NO: 95
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGRGDNWVGFAHWGQ
GTLVTVSS
SEQ ID NO: 96
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGRGDNWVGFANRGQG
TLVTVSS
SEQ ID NO: 97
EVQLVESGGGLVQPGGSLRLSCAASGFTFNTYAMNWVRQAPGKGLEWVARIRSKY
NNYATYYADSVKDRFTISRDDSKNSLYLQMNSLKTEDTAVYYCVRHSNFGNSKVS
WFAYWGQGTLVTVSSGGSGGSGGSGGSGGQAVVTQEPSLTVSPGGTVTLTCRSSAG
AVTTSNYDNWVQQKPGQAPRGLIGGTNKRAPWTPARFSGSLLGGKAALTITGAQAE
DEADYYCALWYSNLWVFGGGTKLTVLSR
SEQ ID NO: 98
DIQMTQSPSSLSASVGDRVTITCSASSSISYMHWYQQKPGQAPRLLIYSTSNLASGVPS
RFSGSGSGTDFTFTISSLEAEDAATYYCHQWSTYHTFGQGTKVEIKRTVAAPSVFIFPP
SDEQLKSGTASVVCLLNNFYPREAKVQWKVDNALQSGNSQESVTEQDSKDSTYSLS
STLTLSKADYEKHKVYACEVTHQGLSSPVTKSFNRGECSRSGGGGEVQLVESGGGL
VQPGGSLRLSCAASGFTFNTYAMNWVRQAPGKGLEWVARIRSKYNNYATYYADSV
KDRFTISRDDSKNSLYLQMNSLKTEDTAVYYCVRHSNFGNSKVSWFAYWGQGTLV
TVSSGGSGGSGGSGGSGGQAVVTQEPSLTVSPGGTVTLTCRSSAGAVTTSNYDNWV
QQKPGQAPRGLIGGTNKRAPWTPARFSGSLLGGKAALTITGAQAEDEADYYCALWY
SNLWVFGGGTKLTVLSR
SEQ ID NO: 99
EVQLVESGGGLVQPGGSLRLSCAASGYTFTSYWMHWIRQSPSRGLEWLGYIRPSTG
YTEYNQKFKDRFTISRDDSKNTAYLQMNSLKTEDTAVYYCGRGDNWVGFKNWGQ
GTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVD
GVEVHNAKTKPREEQYQSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPG
SEQ ID NO: 100
EVQLVESGGGLVQPGGSLRLSCAASGFTFNTYAMNWVRQAPGKGLEWVARIRSKY
NNYATYYADSVKDRFTISRDDSKNSLYLQMNSLKTEDTAVYYCVRHSNFGNSKVS
WFAYWGQGTLVTVSS
SEQ ID NO: 101
QAVVTQEPSLTVSPGGTVTLTCRSSAGAVTTSNYDNWVQQKPGQAPRGLIGGTNKR
APWTPARFSGSLLGGKAALTITGAQAEDEADYYCALWYSNLWVFGGGTKLTVLSR

1. A conditionally active bispecific antibody comprising an IgG antibody or antigen-binding antibody fragment thereof that binds to a human EpCAM protein comprising a light chain variable region having three complementarity determining regions L1, L2 and L3; a heavy chain variable region having three complementarity determining regions H1, H2 and H3; and at least one scFv antibody fragment that binds to a T-lymphocyte protein linked to a C-terminus of at least one light chain of the IgG antibody or antibody fragment; wherein
the IgG light chain variable region is selected from the group consisting of light chain variable regions with L1, L2 and L3 complementarity determining regions having the following amino acid sequences, respectively:
i) SEQ ID NO: 7, SEQ ID NO: 2 and SEQ ID NO: 3;
ii) SEQ ID NO: 8, SEQ ID NO: 2 and SEQ ID NO: 3;
iii) SEQ ID NO: 9, SEQ ID NO: 2 and SEQ ID NO: 3;
iv) SEQ ID NO: 10, SEQ ID NO: 2 and SEQ ID NO: 3;
v) SEQ ID NO: 1, SEQ ID NO: 11 and SEQ ID NO: 3;
vi) SEQ ID NO: 1, SEQ ID NO: 12 and SEQ ID NO: 3;
vii) SEQ ID NO: 1, SEQ ID NO: 13 and SEQ ID NO: 3;
viii) SEQ ID NO: 1, SEQ ID NO: 14 and SEQ ID NO: 3;
ix) SEQ ID NO: 1, SEQ ID NO: 15 and SEQ ID NO: 3;
x) SEQ ID NO: 1, SEQ ID NO: 16 and SEQ ID NO: 3;
xi) SEQ ID NO: 1, SEQ ID NO: 17 and SEQ ID NO: 3;
xii) SEQ ID NO: 1, SEQ ID NO: 18 and SEQ ID NO: 3;
xiii) SEQ ID NO: 1, SEQ ID NO: 19 and SEQ ID NO: 3;
xiv) SEQ ID NO: 1, SEQ ID NO: 20 and SEQ ID NO: 3;
xv) SEQ ID NO: 1, SEQ ID NO: 2 and SEQ ID NO: 22; and
xvi) SEQ ID NO: 1, SEG ID NO. 2 and SEQ ID NO: 23; and
the IgG heavy chain variable region complementarity determining regions H1, H2 and H3 have amino acid sequences SEQ ID NO: 4, SEQ ID NO: 5 and SEQ ID NO: 6, respectively or
the IgG heavy chain variable region is selected from the group consisting of heavy chain variable regions with H1, H2 and H3 complementarity determining regions having amino acid sequences, respectively:
i) SEQ ID NO: 24, SEQ ID NO: 5 and SEQ ID NO: 6;
ii) SEQ ID NO: 25, SEQ ID NO: 5 and SEQ ID NO: 6;
iii) SEQ ID NO: 26, SEQ ID NO: 5 and SEQ ID NO: 6;
iv) SEQ ID NO: 27, SEQ ID NO: 5 and SEQ ID NO: 6;
v) SEQ ID NO: 28, SEQ ID NO: 5 and SEQ ID NO: 6;
vi) SEQ ID NO: 29, SEQ ID NO: 5 and SEQ ID NO: 6;
vii) SEQ ID NO: 30, SEQ ID NO: 5 and SEQ ID NO: 6;
viii) SEQ ID NO: 31, SEQ ID NO: 5 and SEQ ID NO: 6;
ix) SEQ ID NO: 4, SEQ ID NO: 32 and SEQ ID NO: 6;
x) SEQ ID NO: 4, SEQ ID NO: 33 and SEQ ID NO: 6;
xi SEQ ID NO: 4, SEQ ID NO: 34 and SEQ ID NO: 6;
xii) SEQ ID NO: 4, SEQ ID NO: 35 and SEQ ID NO: 6;
xiii) SEQ ID NO: 4, SEQ ID NO: 36 and SEQ ID NO: 6;
xiv) SEQ ID NO: 4, SEQ ID NO: 37 and SEQ ID NO: 6;
xv) SEQ ID NO: 4, SEQ ID NO: 38 and SEQ ID NO: 6;
xvi) SEQ ID NO: 4, SEQ ID NO: 5 and SEQ ID NO: 39;
xvii) SEQ ID NO: 4, SEQ ID NO: 5 and SEQ ID NO: 40;
xviii) SEQ ID NO: 4, SEQ ID NO: 5 and SEQ ID NO: 41;
xix) SEQ ID NO: 4, SEQ ID NO: 5 and SEQ ID NO: 42;
xx) SEQ ID NO: 4, SEQ ID NO: 5 and SEQ ID NO: 43;
xxi) SEQ ID NO: 4, SEQ ID NO: 5 and SEQ ID NO: 44;
xxii) SEQ ID NO: 4, SEQ ID NO: 5 and SEQ ID NO: 45;
xxiii) SEQ ID NO: 4, SEQ ID NO: 5 and SEQ ID NO: 46;
xxiv) SEQ ID NO: 4, SEQ ID NO: 5 and SEQ ID NO: 47;
xxv) SEQ ID NO: 4, SEQ ID NO: 5 and SEQ ID NO: 48;
xxvi) SEQ ID NO: 4, SEQ ID NO: 5 and SEQ ID NO: 49; and
xxvii) SEQ ID NO: 4, SEQ ID NO: 5 and SEQ ID NO: 50; and
the IgG light chain variable region complementarity determining regions L1, L2 and L3 have the following amino acid sequences SEQ ID NO: 1, SEQ ID NO: 2 and SEQ ID NO: 3, respectively.

2. (canceled)

3. The conditionally active bispecific antibody of claim 1, wherein the IgG heavy chain variable region has an amino acid sequence of SEQ ID NO; 52 and the IgG light chain variable region has an amino acid sequence selected from the group consisting of SEQ ID NOS: 53-69.

4. The conditionally active bispecific antibody of claim 1, wherein the IgG light chain variable region has an amino acid sequence of SEQ ID NO: 51 and the IgG heavy chain variable region has an amino acid sequence selected from the group consisting of SEQ ID NOS: 70-96.

5. The conditionally active bispecific antibody of claim 4, wherein the IgG light chain variable region complementarity determining regions L1, L2 and L3 have amino acid sequences of SEQ ID NO: 1, SEQ ID NO: 2 and SEQ ID NO:3, respectively, and the IgG heavy chain complementarity determining regions H1, H2 and H3 have amino acid sequences of SEQ NO: 4, SEQ ID: 5 and SEQ ID NO: 45, respectively.

6. (canceled)

7. (canceled)

8. The conditionally active bispecific antibody of claim 1, wherein the IgG antibody or antigen binding antibody fragment thereof has a higher binding affinity to an EpCAM protein at a tumor microenvironment pH of 5.0 to 6.9 as compared to binding affinity to the EpCAM protein at a non-tumor microenvironment pH of 7.0 to 7.6.

9. The conditionally active bispecific antibody of claim 8, wherein the IgG antibody or antigen binding antibody fragment thereof has a ratio of binding affinity to a human EpCAM protein at a pH of 6.0 to a binding affinity to the human EpCAM protein at a pH of 7.4 of at least about 1.5:1.

10. The conditionally active bispecific antibody of claim 9, wherein the IgG antibody or antigen-binding antibody fragment thereof has a ratio of binding affinity to a human EpCAM protein at a pH of 6.0 to a binding affinity to the human EpCAM protein at a pH of 7.4 of at least about 6:1.

11. The conditionally active bispecific antibody of claim 1, wherein the scFv antibody fragment binds to a CD3 protein.

12. The conditionally active bispecific antibody of claim 11, wherein the scFv antibody fragment is a conditionally active scFv antibody fragment that has a greater binding affinity to the CD3 protein at a tumor microenvironment pH of 5.0 to 6.9 as compared to binding affinity to the CD3 protein at a non-tumor microenvironment pH of 7.0 to 7.6.

13. The conditionally active bispecific antibody of claim 12, wherein the scFv antibody fragment is obtained from a non-conditionally active parental anti-CD3 antibody.

14. The conditionally active bispecific antibody of claim 13, wherein the conditionally active scFv fragment has a lower binding affinity to the CD3 protein at the non-tumor microenvironment pH of 7.0-7.6 as compared to a binding affinity of the parental antibody to the CD3 protein at the non-tumor microenvironment pH of 7.0-7.6.

15. The conditionally active bispecific antibody of claim 12, wherein the scFv antibody fragment has an amino acid sequence of SEQ ID NO: 97.

16. The conditionally active bispecific antibody of claim 8, wherein the bispecific antibody comprises a light chain having an amino acid sequence of SEQ ID NO: 98 and a heavy chain having an amino acid sequence of SEQ ID NO: 99.

17. The conditionally active bispecific antibody of claim 12, wherein the conditionally active scFv antibody fragment has a greater binding affinity to the CD3 protein at a tumor microenvironment pH of 6.0 as compared to a binding affinity to the CD3 protein at a non-tumor microenvironment pH of 7.4.

18. The conditionally active bispecific antibody of claim 1, wherein the IgG antibody or antigen binding antibody fragment thereof has a ratio of binding affinity to a cynomolgus EpCAM protein at a pH of 6.0 to a binding affinity to the cynomolgus EpCAM protein at a pH of 7.4 of at least about 1.5:1.

19. The conditionally active bispecific antibody of claim 18, wherein the IgG antibody or antigen binding antibody fragment thereof has a ratio of binding affinity to a cynomolgus EpCAM protein at a pH of 6.0 to a binding affinity to the cynomolgus EpCAM protein at a pH of 7.4 of at least about 6:1.

20. A conditionally active antibody or antigen binding antibody fragment thereof that binds to a human EpCAM protein comprising a light chain variable region having three complementarity determining regions L1, L2 and L3; and a heavy chain variable region having three complementarity determining regions H1, H2 and H3; wherein
the light chain variable region is selected from the group consisting of light chain variable regions with L1, L2 and L3 complementarity determining regions having amino acid sequences for L1, L2 and L3, respectively, of:
i) SEQ ID NO: 7, SEQ ID NO: 2 and SEQ ID NO: 3;
ii) SEQ ID NO: 8, SEQ ID NO: 2 and SEQ ID NO: 3;
iii) SEQ ID NO: 9, SEQ ID NO: 2 and SEQ ID NO: 3;
iv) SEQ ID NO: 10, SEQ ID NO: 2 and SEQ ID NO: 3;
v) SEQ ID NO: 1, SEQ ID NO: 11 and SEQ ID NO: 3;
vi) SEQ ID NO: 1, SEQ ID NO: 12 and SEQ ID NO: 3;
vii) SEQ ID NO: 1, SEQ ID NO: 13 and SEQ ID NO: 3;
viii) SEQ ID NO: 1, SEQ ID NO: 14 and SEQ ID NO: 3;
ix) SEQ ID NO: 1, SEQ ID NO: 15 and SEQ ID NO: 3;
x) SEQ ID NO: 1, SEQ ID NO: 16 and SEQ ID NO: 3;
xi) SEQ ID NO: 1, SEQ ID NO: 17 and SEQ ID NO: 3;
xii) SEQ ID NO: 1, SEQ ID NO: 18 and SEQ ID NO: 3;
xiii) SEQ ID NO: 1, SEQ ID NO: 19 and SEQ ID NO: 3;
xiv) SEQ ID NO: 1, SEQ ID NO: 20 and SEQ ID NO: 3
xv) SEQ ID NO: 1, SEQ ID NO: 2 and SEQ ID NO: 22; and
xvi) SEQ ID NO: 1, SEQ ID NO. 2 and SEQ ID NO: 23; and
the heavy chain variable region includes three complementarity determining regions H1, H2 and H3 having amino acid sequences of SEQ ID NO: 4, SEQ ID NO: 5 and SEQ ID NO:6, respectively, or
the heavy chain variable region is selected from the group consisting of heavy chain variable regions with H1, H2 and H3 complementarity determining regions having amino acid sequences for H1, H2 and H3, respectively, of:
i) SEQ ID NO: 24, SEQ ID NO: 5 and SEQ ID NO: 6;
ii) SEQ ID NO: 25, SEQ ID NO: 5 and SEQ ID NO: 6;
iii) SEQ ID NO: 26, SEQ ID NO: 5 and SEQ ID NO: 6;
iv) SEQ ID NO: 27, SEQ ID NO: 5 and SEQ ID NO: 6;
v) SEQ ID NO: 28, SEQ ID NO: 5 and SEQ ID NO: 6;
vi) SEQ ID NO: 29, SEQ ID NO: 5 and SEQ ID NO: 6;
vii) SEQ ID NO: 30, SEQ ID NO: 5 and SEQ ID NO: 6;
viii) SEQ ID NO: 31, SEQ ID NO: 5 and SEQ ID NO: 6;
ix) SEQ ID NO: 4, SEQ ID NO: 32 and SEQ ID NO: 6;
x) SEQ ID NO: 4, SEQ ID NO: 33 and SEQ ID NO: 6;
xi SEQ ID NO: 4, SEQ ID NO: 34 and SEQ ID NO: 6;
xii) SEQ ID NO: 4, SEQ ID NO: 35 and SEQ ID NO: 6;
xiii) SEQ ID NO: 4, SEQ ID NO: 36 and SEQ ID NO: 6;
xiv) SEQ ID NO: 4, SEQ ID NO: 37 and SEQ ID NO: 6;
xv) SEQ ID NO: 4, SEQ ID NO: 38 and SEQ ID NO: 6;
xvi) SEQ ID NO: 4, SEQ ID NO: 5 and SEQ ID NO: 39;
xvii) SEQ ID NO: 4, SEQ ID NO: 5 and SEQ ID NO: 40;
xviii) SEQ ID NO: 4, SEQ ID NO: 5 and SEQ ID NO: 41;
xix) SEQ ID NO: 4, SEQ ID NO: 5 and SEQ ID NO: 42;
xx) SEQ ID NO: 4, SEQ ID NO: 5 and SEQ ID NO: 43;
xxi) SEQ ID NO: 4, SEQ ID NO: 5 and SEQ ID NO: 44;
xxii) SEQ ID NO: 4, SEQ ID NO: 5 and SEQ ID NO: 45;
xxiii) SEQ ID NO: 4, SEQ ID NO: 5 and SEQ ID NO: 46;
xxiv) SEQ ID NO: 4, SEQ ID NO: 5 and SEQ ID NO: 47;
xxv) SEQ ID NO: 4, SEQ ID NO: 5 and SEQ ID NO: 48;
xxvi) SEQ ID NO: 4, SEQ ID NO: 5 and SEQ ID NO: 49; and
xxvii) SEQ ID NO: 4, SEQ ID NO: 5 and SEQ ID NO: 50; and
the light chain variable region complementarity determining regions L1, L2 and L3 have amino acid sequences of SEQ ID NO: 1, SEQ ID NO: 2 and SEQ ID NO: 3, respectively.

21. (canceled)

22. The conditionally active antibody or antigen binding antibody fragment thereof of claim 20, wherein the heavy chain variable region has an amino acid sequence of SEQ ID NO; 52 and the light chain variable region has an amino acid sequence selected from the group consisting of SEQ ID NOS: 53-69.

23. The conditionally active antibody or antigen binding antibody fragment thereof of claim 20, wherein the light chain variable region has an amino acid sequence of SEQ ID NO: 51 and the heavy chain variable region has an amino acid sequence selected from the group consisting of SEQ ID NOS: 70-96.

24. (canceled)

25. The conditionally active antibody or antigen binding antibody fragment thereof of claim 20, wherein the antibody or antigen binding antibody fragment thereof has a higher binding affinity to a human EpCAM protein at a tumor microenvironment pH of 5.0 to 6.9 than a binding affinity to the human EpCAM protein at a non-tumor microenvironment pH of 7.0 to 7.6.

26. (canceled)

27. (canceled)

28. The conditionally active antibody or antigen binding antibody fragment thereof of claim 20, wherein the antibody or antigen binding antibody fragment thereof has a ratio of binding affinity to a human EpCAM protein at a pH of 6.0 to a binding affinity to the human EpCAM protein at a pH of 7.4 of at least about 1.5:1.

29. (canceled)

30. (canceled)

31. (canceled)

32. (canceled)


SEQUENCE LISTING (101 sequences)

SEQ ID NO: 1 - protein, 10 aa, synthetic construct
SASSSISYMH

SEQ ID NO: 2 - protein, 7 aa, synthetic construct
STSNLAS

SEQ ID NO: 3 - protein, 8 aa, synthetic construct
HQWSTYHT

SEQ ID NO: 4 - protein, 10 aa, synthetic construct
GYTFTSYWMH

SEQ ID NO: 5 - protein, 17 aa, synthetic construct
YIRPSTGYTEYNQKFKD

SEQ ID NO: 6 - protein, 9 aa, synthetic construct
GDNWVGFAN

SEQ ID NO: 7 - protein, 10 aa, synthetic construct
SASSSISDMH

SEQ ID NO: 8 - protein, 10 aa, synthetic construct
SASSSISEMH

SEQ ID NO: 9 - protein, 10 aa, synthetic construct
SASSSISYPH

SEQ ID NO: 10 - protein, 10 aa, synthetic construct
SASSSISYMI

SEQ ID NO: 11 - protein, 7 aa, synthetic construct
DTSNLAS

SEQ ID NO: 12 - protein, 7 aa, synthetic construct
ETSNLAS

SEQ ID NO: 13 - protein, 7 aa, synthetic construct
HTSNLAS

SEQ ID NO: 14 - protein, 7 aa, synthetic construct
KTSNLAS

SEQ ID NO: 15 - protein, 7 aa, synthetic construct
QTSNLAS

SEQ ID NO: 16 - protein, 7 aa, synthetic construct
SNSNLAS

SEQ ID NO: 17 - protein, 7 aa, synthetic construct
STSELAS

SEQ ID NO: 18 - protein, 7 aa, synthetic construct
STSNDAS

SEQ ID NO: 19 - protein, 7 aa, synthetic construct
STSNLKS

SEQ ID NO: 20 - protein, 7 aa, synthetic construct
STSNLAE

SEQ ID NO: 21 - protein, 8 aa, synthetic construct
EQWSTYHT

SEQ ID NO: 22 - protein, 8 aa, synthetic construct
HGWSTYHT

SEQ ID NO: 23 - protein, 8 aa, synthetic construct
HQWSTYKT

SEQ ID NO: 24 - protein, 10 aa, synthetic construct
GYNFTSYWMH

SEQ ID NO: 25 - protein, 10 aa, synthetic construct
GYTETSYWMH

SEQ ID NO: 26 - protein, 10 aa, synthetic construct
GYTHTSYWMH

SEQ ID NO: 27 - protein, 10 aa, synthetic construct
GYTKTSYWMH

SEQ ID NO: 28 - protein, 10 aa, synthetic construct
GYTFTSYHMH

SEQ ID NO: 29 - protein, 10 aa, synthetic construct
GYTFTSYWDH

SEQ ID NO: 30 - protein, 10 aa, synthetic construct
GYTFTSYWEH

SEQ ID NO: 31 - protein, 10 aa, synthetic construct
GYTFTSYWMW

SEQ ID NO: 32 - protein, 17 aa, synthetic construct
HIRPSTGYTEYNQKFKD

SEQ ID NO: 33 - protein, 34 aa, synthetic construct
YDRPSTGYTEYNQKFKDYDRPSTGYTEYNQKFKD

SEQ ID NO: 34 - protein, 17 aa, synthetic construct
YERPSTGYTEYNQKFKD

SEQ ID NO: 35 - protein, 17 aa, synthetic construct
YIGPSTGYTEYNQKFKD

SEQ ID NO: 36 - protein, 17 aa, synthetic construct
YIHPSTGYTEYNQKFKD

SEQ ID NO: 37 - protein, 17 aa, synthetic construct
YIRLSTGYTEYNQKFKD

SEQ ID NO: 38 - protein, 17 aa, synthetic construct
YIRWSTGYTEYNQKFKD

SEQ ID NO: 39 - protein, 11 aa, synthetic construct
ERGDNWVGFAN

SEQ ID NO: 40 - protein, 10 aa, synthetic construct
WGDNWVGFAN

SEQ ID NO: 41 - protein, 10 aa, synthetic construct
YGDNWVGFAN

SEQ ID NO: 42 - protein, 9 aa, synthetic construct
GQNWVGFAN

SEQ ID NO: 43 - protein, 9 aa, synthetic construct
GDNWKGFAN

SEQ ID NO: 44 - protein, 9 aa, synthetic construct
GDNWVGMAN

SEQ ID NO: 45 - protein, 9 aa, synthetic construct
GDNWVGFKN

SEQ ID NO: 46 - protein, 9 aa, synthetic construct
GDNWVGFRN

SEQ ID NO: 47 - protein, 9 aa, synthetic construct
GDNWVGFAD

SEQ ID NO: 48 - protein, 9 aa, synthetic construct
GDNWVGFAE

SEQ ID NO: 49 - protein, 9 aa, synthetic construct
GDNWVGFAH

SEQ ID NO: 50 - protein, 10 aa, synthetic construct
GDNWVGFANR

SEQ ID NO: 51 - protein, 105 aa, synthetic construct
DIQMTQSPSSLSASVGDRVTITCSASSSISYMHWYQQKPGQAPRLLIYSTSNLASGVPSRFSGSGSGTDFTFTISSLEAEDAATYYCHQWSTYHTFGQGTKVEIK

SEQ ID NO: 52 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGRGDNWVGFANWGQGTLVTVSS

SEQ ID NO: 53 - protein, 105 aa, synthetic construct
DIQMTQSPSSLSASVGDRVTITCSASSSISDMHWYQQKPGQAPRLLIYSTSNLASGVPSRFSGSGSGTDFTFTISSLEAEDAATYYCHQWSTYHTFGQGTKVEIK

SEQ ID NO: 54 - protein, 105 aa, synthetic construct
DIQMTQSPSSLSASVGDRVTITCSASSSISEMHWYQQKPGQAPRLLIYSTSNLASGVPSRFSGSGSGTDFTFTISSLEAEDAATYYCHQWSTYHTFGQGTKVEIK

SEQ ID NO: 55 - protein, 105 aa, synthetic construct
DIQMTQSPSSLSASVGDRVTITCSASSSISYPHWYQQKPGQAPRLLIYSTSNLASGVPSRFSGSGSGTDFTFTISSLEAEDAATYYCHQWSTYHTFGQGTKVEIK

SEQ ID NO: 56 - protein, 105 aa, synthetic construct
DIQMTQSPSSLSASVGDRVTITCSASSSISYMIWYQQKPGQAPRLLIYSTSNLASGVPSRFSGSGSGTDFTFTISSLEAEDAATYYCHQWSTYHTFGQGTKVEIK

SEQ ID NO: 57 - protein, 105 aa, synthetic construct
DIQMTQSPSSLSASVGDRVTITCSASSSISYMHWYQQKPGQAPRLLIYDTSNLASGVPSRFSGSGSGTDFTFTISSLEAEDAATYYCHQWSTYHTFGQGTKVEIK

SEQ ID NO: 58 - protein, 105 aa, synthetic construct
DIQMTQSPSSLSASVGDRVTITCSASSSISYMHWYQQKPGQAPRLLIYETSNLASGVPSRFSGSGSGTDFTFTISSLEAEDAATYYCHQWSTYHTFGQGTKVEIK

SEQ ID NO: 59 - protein, 105 aa, synthetic construct
DIQMTQSPSSLSASVGDRVTITCSASSSISYMHWYQQKPGQAPRLLIYHTSNLASGVPSRFSGSGSGTDFTFTISSLEAEDAATYYCHQWSTYHTFGQGTKVEIK

SEQ ID NO: 60 - protein, 105 aa, synthetic construct
DIQMTQSPSSLSASVGDRVTITCSASSSISYMHWYQQKPGQAPRLLIYKTSNLASGVPSRFSGSGSGTDFTFTISSLEAEDAATYYCHQWSTYHTFGQGTKVEIK

SEQ ID NO: 61 - protein, 105 aa, synthetic construct
DIQMTQSPSSLSASVGDRVTITCSASSSISYMHWYQQKPGQAPRLLIYQTSNLASGVPSRFSGSGSGTDFTFTISSLEAEDAATYYCHQWSTYHTFGQGTKVEIK

SEQ ID NO: 62 - protein, 105 aa, synthetic construct
DIQMTQSPSSLSASVGDRVTITCSASSSISYMHWYQQKPGQAPRLLIYSNSNLASGVPSRFSGSGSGTDFTFTISSLEAEDAATYYCHQWSTYHTFGQGTKVEIK

SEQ ID NO: 63 - protein, 105 aa, synthetic construct
DIQMTQSPSSLSASVGDRVTITCSASSSISYMHWYQQKPGQAPRLLIYSTSELASGVPSRFSGSGSGTDFTFTISSLEAEDAATYYCHQWSTYHTFGQGTKVEIK

SEQ ID NO: 64 - protein, 105 aa, synthetic construct
DIQMTQSPSSLSASVGDRVTITCSASSSISYMHWYQQKPGQAPRLLIYSTSNDASGVPSRFSGSGSGTDFTFTISSLEAEDAATYYCHQWSTYHTFGQGTKVEIK

SEQ ID NO: 65 - protein, 105 aa, synthetic construct
DIQMTQSPSSLSASVGDRVTITCSASSSISYMHWYQQKPGQAPRLLIYSTSNLKSGVPSRFSGSGSGTDFTFTISSLEAEDAATYYCHQWSTYHTFGQGTKVEIK

SEQ ID NO: 66 - protein, 105 aa, synthetic construct
DIQMTQSPSSLSASVGDRVTITCSASSSISYMHWYQQKPGQAPRLLIYSTSNLAEGVPSRFSGSGSGTDFTFTISSLEAEDAATYYCHQWSTYHTFGQGTKVEIK

SEQ ID NO: 67 - protein, 105 aa, synthetic construct
DIQMTQSPSSLSASVGDRVTITCSASSSISYMHWYQQKPGQAPRLLIYSTSNLASGVPSRFSGSGSGTDFTFTISSLEAEDAATYYCEQWSTYHTFGQGTKVEIK

SEQ ID NO: 68 - protein, 105 aa, synthetic construct
DIQMTQSPSSLSASVGDRVTITCSASSSISYMHWYQQKPGQAPRLLIYSTSNLASGVPSRFSGSGSGTDFTFTISSLEAEDAATYYCHGWSTYHTFGQGTKVEIK

SEQ ID NO: 69 - protein, 105 aa, synthetic construct
DIQMTQSPSSLSASVGDRVTITCSASSSISYMHWYQQKPGQAPRLLIYSTSNLASGVPSRFSGSGSGTDFTFTISSLEAEDAATYYCHQWSTYKTFGQGTKVEIK

SEQ ID NO: 70 - protein, 118 aa, synthetic construct
EVQLVESGGGLVQPGGSLRLSCAASGYNFTSYWMHWIRQSPSRGLEWLGYIRPSTGYTEYNQKFKDRFTISRDDSKNTAYLQMNSLKTEDTAVYYCGRGDNWVGFANWGQGTLVTVSS

SEQ ID NO: 71 - protein, 118 aa, synthetic construct
EVQLVESGGGLVQPGGSLRLSCAASGYTETSYWMHWIRQSPSRGLEWLGYIRPSTGYTEYNQKFKDRFTISRDDSKNTAYLQMNSLKTEDTAVYYCGRGDNWVGFANWGQGTLVTVSS

SEQ ID NO: 72 - protein, 118 aa, synthetic construct
EVQLVESGGGLVQPGGSLRLSCAASGYTHTSYWMHWIRQSPSRGLEWLGYIRPSTGYTEYNQKFKDRFTISRDDSKNTAYLQMNSLKTEDTAVYYCGRGDNWVGFANWGQGTLVTVSS

SEQ ID NO: 73 - protein, 118 aa, synthetic construct
EVQLVESGGGLVQPGGSLRLSCAASGYTKTSYWMHWIRQSPSRGLEWLGYIRPSTGYTEYNQKFKDRFTISRDDSKNTAYLQMNSLKTEDTAVYYCGRGDNWVGFANWGQGTLVTVSS

SEQ ID NO: 74 - protein, 118 aa, synthetic construct
EVQLVESGGGLVQPGGSLRLSCAASGYTFTSYHMHWIRQSPSRGLEWLGYIRPSTGYTEYNQKFKDRFTISRDDSKNTAYLQMNSLKTEDTAVYYCGRGDNWVGFANWGQGTLVTVSS

SEQ ID NO: 75 - protein, 118 aa, synthetic construct
EVQLVESGGGLVQPGGSLRLSCAASGYTFTSYWDHWIRQSPSRGLEWLGYIRPSTGYTEYNQKFKDRFTISRDDSKNTAYLQMNSLKTEDTAVYYCGRGDNWVGFANWGQGTLVTVSS

SEQ ID NO: 76 - protein, 118 aa, synthetic construct
EVQLVESGGGLVQPGGSLRLSCAASGYTFTSYWEHWIRQSPSRGLEWLGYIRPSTGYTEYNQKFKDRFTISRDDSKNTAYLQMNSLKTEDTAVYYCGRGDNWVGFANWGQGTLVTVSS

SEQ ID NO: 77 - protein, 118 aa, synthetic construct
EVQLVESGGGLVQPGGSLRLSCAASGYTFTSYWMWWIRQSPSRGLEWLGYIRPSTGYTEYNQKFKDRFTISRDDSKNTAYLQMNSLKTEDTAVYYCGRGDNWVGFANWGQGTLVTVSS

SEQ ID NO: 78 - protein, 118 aa, synthetic construct
EVQLVESGGGLVQPGGSLRLSCAASGYTFTSYWMHWIRQSPSRGLEWLGHIRPSTGYTEYNQKFKDRFTISRDDSKNTAYLQMNSLKTEDTAVYYCGRGDNWVGFANWGQGTLVTVSS

SEQ ID NO: 79 - protein, 118 aa, synthetic construct
EVQLVESGGGLVQPGGSLRLSCAASGYTFTSYWMHWIRQSPSRGLEWLGYDRPSTGYTEYNQKFKDRFTISRDDSKNTAYLQMNSLKTEDTAVYYCGRGDNWVGFANWGQGTLVTVSS

SEQ ID NO: 80 - protein, 118 aa, synthetic construct
EVQLVESGGGLVQPGGSLRLSCAASGYTFTSYWMHWIRQSPSRGLEWLGYERPSTGYTEYNQKFKDRFTISRDDSKNTAYLQMNSLKTEDTAVYYCGRGDNWVGFANWGQGTLVTVSS

SEQ ID NO: 81 - protein, 118 aa, synthetic construct
EVQLVESGGGLVQPGGSLRLSCAASGYTFTSYWMHWIRQSPSRGLEWLGYIGPSTGYTEYNQKFKDRFTISRDDSKNTAYLQMNSLKTEDTAVYYCGRGDNWVGFANWGQGTLVTVSS

SEQ ID NO: 82 - protein, 118 aa, synthetic construct
EVQLVESGGGLVQPGGSLRLSCAASGYTFTSYWMHWIRQSPSRGLEWLGYIHPSTGYTEYNQKFKDRFTISRDDSKNTAYLQMNSLKTEDTAVYYCGRGDNWVGFANWGQGTLVTVSS

SEQ ID NO: 83 - protein, 118 aa, synthetic construct
EVQLVESGGGLVQPGGSLRLSCAASGYTFTSYWMHWIRQSPSRGLEWLGYIRLSTGYTEYNQKFKDRFTISRDDSKNTAYLQMNSLKTEDTAVYYCGRGDNWVGFANWGQGTLVTVSS

SEQ ID NO: 84 - protein, 118 aa, synthetic construct
EVQLVESGGGLVQPGGSLRLSCAASGYTFTSYWMHWIRQSPSRGLEWLGYIRWSTGYTEYNQKFKDRFTISRDDSKNTAYLQMNSLKTEDTAVYYCGRGDNWVGFANWGQGTLVTVSS

SEQ ID NO: 85 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCERGDNWVGFANWGQGTLVTVSS

SEQ ID NO: 86 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGWGDNWVGFANWGQGTLVTVSS

SEQ ID NO: 87 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGYGDNWVGFANWGQGTLVTVSS

SEQ ID NO: 88 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGRGQNWVGFANWGQGTLVTVSS

SEQ ID NO: 89 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGRGDNWKGFANWGQGTLVTVSS

SEQ ID NO: 90 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGRGDNWVGMANWGQGTLVTVSS

SEQ ID NO: 91 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGRGDNWVGFKNWGQGTLVTVSS

SEQ ID NO: 92 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGRGDNWVGFRNWGQGTLVTVSS

SEQ ID NO: 93 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGRGDNWVGFADWGQGTLVTVSS

SEQ ID NO: 94 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGRGDNWVGFAEWGQGTLVTVSS

SEQ ID NO: 95 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGRGDNWVGFAHWGQGTLVTVSS

SEQ ID NO: 96 - protein, 118 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGRGDNWVGFANRGQGTLVTVSS

SEQ ID NO: 97 - protein, 250 aa, synthetic construct
EVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHSNFGNSKVSWFAYWGQGTLVTVSSGGSGGSGGSGGSGGQAVVTQEPSLTVSPGGTVTLTCRSSAGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 98 - protein, 469 aa, synthetic construct
DIQMTQSPSSLSASVGDRVTITCSASSSISYMHWYQQKPGQAPRLLIYSTSNLASGVPSRFSGSGSGTDFTFTISSLEAEDAATYYCHQWSTYHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHSNFGNSKVSWFAYWGQGTLVTVSSGGSGGSGGSGGSGGQAVVTQEPSLTVSPGGTVTLTCRSSAGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 99 - protein, 447 aa, synthetic construct
EVQLVESGGGLVQPGGSLRLSCAASGYTFTSYWMHWIRQSPSRGLEWLGYIRPSTGYTEYNQKFKDRFTISRDDSKNTAYLQMNSLKTEDTAVYYCGRGDNWVGFKNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 125 aa, synthetic construct
EVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHSNFGNSKVSWFAYWGQGTLVTVSS

SEQ ID NO: 101 - protein, 111 aa, synthetic construct
QAVVTQEPSLTVSPGGTVTLTCRSSAGAVTTSNYDNWVQQKPGQAPRGLIGGTNKRAPWTPARFSGSLLGGKAALTITGAQAEDEADYYCALWYSNLWVFGGGTKLTVLSR